(19) United States
(12) Patent Application Publication
(10) Pub. No.: US 2021/0040041 A1
(43) Pub. Date: Feb. 11, 2021

(54) QUINOLINE DERIVATIVES AND PREPARATION METHODS AND USES THEREOF

(71) Applicant: DONGGUAN HEC PESTICIDES R&D CO., LTD., Dongguan, Guangdong (JP)

(72) Inventors: Yitao LI, Dongguan (CN); Jian LIN, Dongguan (CN); Wenqiang YAO, Dongguan (CN); Faping WANG, Dongguan (CN); Falin LI, Dongguan (CN); Chuanwei WANG, Dongguan (CN)

(73) Assignee: DONGGUAN HEC PESTICIDES R&D CO., LTD., Dongguan, Guangdong (CN)

(21) Appl. No.: 17/047,944

(22) PCT Filed: Apr. 30, 2019
(86) PCT No.: PCT/CN2019/085094

§ 371 Date: Oct. 15, 2020

(30) Foreign Application Priority Data
May 4, 2018 (CN) 201810417184.X

(51) Int. Cl. C07D 215/233; C07D 405/12; A01N 43/42

(52) U.S. Cl. CPC C07D 215/233; A01N 43/42; C07D 405/12

(57) ABSTRACT

A quinoline derivative, and preparation methods and uses thereof; specifically, a quinoline derivative of Formula (I) or a stereoisomer, an N-oxide and a salt thereof, their preparation methods, and their uses as pesticides, forms of a pesticide composition thereof, and methods of controlling pests by using these compounds or compositions in agriculture or gardens; wherein R1, R2, R3, R4, R5, RA, RB, RC, RC1, RC2, R6, R7, R8, R9, R10, R11, Rx and n have the definitions as described herein.

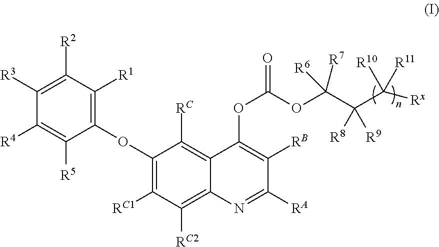

### CROSS-REFERENCE TO RELATED APPLICATIONS

This application claims priority to Chinese Patent Application Serial No 201810417184.X, filed on May 4, 2018, which is hereby incorporated by reference in its entirety.

### FIELD OF THE INVENTION

The invention pertains to the field of agricultural pest control, and relates to quinoline derivatives and N-oxides and salts thereof, which are used for controlling pests.

### BACKGROUND OF THE INVENTION

At present, although there are many methods for controlling pests in agriculture, novel, more effective and reasonable pesticidal compounds are still being explored and discovered. The present invention provides a novel type of compounds that can be further studied. Quinoline compounds have excellent biological and physiological activities. In the field of medical care and plant protection, quinoline compounds have shown wide applications and development prospects, especially in plant protection. These compounds have different mechanisms of action with most pesticides used in pest control, which can solve the increasingly serious problem of pesticide resistance. Because of this, quinoline compounds have opened up a new field for the development of agricultural chemicals, and provided a new way to find novel agricultural chemicals with high efficiency and low toxicity.

### SUMMARY OF THE INVENTION

The present invention provides a quinoline derivative and compositions comprising such derivative, the quinoline derivative and the compositions are used for controlling pests in agriculture or gardens. In particular, the compounds have very good control effects on Lepidoptera pests such as armyworm, diamondback moth, Spodoptera exigua and Prodenia litura, and have low toxicity and are targeted pesticides, which are expected to solve the problem of low efficiency and high toxicity of insecticides.

Specifically,

In one aspect, provided herein is a compound of Formula (I) or a stereoisomer, an N-oxide or an acceptable salt thereof:

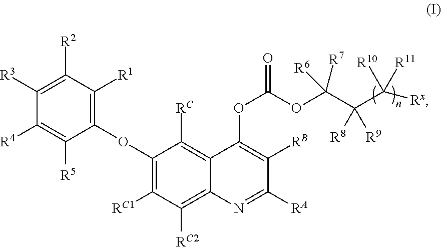

wherein

R^{x} is

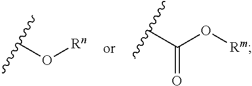

Rⁿ is alkyl, alkenyl, alkynyl, aryl or arylalkyl; wherein R is optionally substituted with 1, 2, 3, 4, 5, 6, 7 or 8 substituents selected from A1;

R^{m} is alkyl, alkenyl or alkynyl; wherein R^{m} is optionally substituted with 1, 2, 3, 4, 5, 6, 7 or 8 substituents selected from A2;

each of A1 and A2 is independently halo, hydroxy, cyano, nitro, amino, alkyl, alkoxy, haloalkyl or haloalkoxy;

each of R¹, R², R³, R⁴ and R⁵ is independently hydrogen, halo, hydroxy, cyano, nitro, amino, carboxy, —C(═O)—NR^{a}R^{b}, —R^{c}—C(═O)—R^{d}, —NR(OR), alkyl, alkenyl, alkynyl, haloalkyl, haloalkenyl, haloalkynyl, hydroxy-substituted alkyl, amino-substituted alkyl, cyano-substituted alkyl, alkyl-SO₂—, alkyl-(C═O)—, alkyl-(C═O)—O—, alkoxy, alkoxy-(C═O)—, alkylthio, alkenyloxy, haloalkoxy, haloalkylthio, haloalkenyloxy, hydroxy-substituted alkoxy, amino-substituted alkoxy, cyano-substituted alkoxy or alkylamino;

each of R^{a}, R^{b}, R^{c}, R^{d}, R^{e} and R^{f} is independently hydrogen or alkyl;

or R¹ and R², or R² and R³, or R³ and R⁴, or R⁴ and R⁵ together form —O—(CH₂)ₘ—O—, —(CH₂)ₘ₁—O— or —(CH₂)ₘ₂—, wherein each of —O—(CH₂)ₘ—O—, —(CH₂)ₘ₁—O— and —(CH₂)ₘ₂— is optionally and independently substituted with 1, 2, 3, 4, 5 or 6 halo;

wherein each of m, m1 and m2 is independently 1, 2 or 3;

each of R^{A} and R^{B} is independently hydrogen, alkyl or haloalkyl;

each of R^{C}, R^{C1} and R^{C2} is independently hydrogen, halo or alkyl;

each of R⁶, R⁷, R⁸, R⁹, R¹⁰ and R¹¹ is independently hydrogen, alkyl, haloalkyl, alkoxy or haloalkoxy;

or Rⁿ and R⁸, together with the atoms to which they are attached, form a 3-8 membered heterocycle; the 3-8 membered heterocycle is optionally substituted with 1, 2, 3, 4, 5, 6, 7 or 8 substituents selected from A3;

wherein each A3 is independently halo, oxo, hydroxy, cyano, nitro, alkyl, alkoxy, haloalkyl or haloalkoxy; and

n is 0, 1, 2 or 3.

In some embodiments, Rⁿ is C₁₋₆ alkyl, C₂₋₈ alkenyl, C₂₋₈ alkynyl, C₆₋₁₄ aryl or C₆₋₁₄ aryl-C₁₋₆ alkyl-; wherein Rⁿ is optionally substituted with 1, 2, 3, 4, 5, 6, 7 or 8 substituents selected from A1;

R^{m} is C₁₋₆ alkyl, C₂₋₈ alkenyl or C₂₋₈ alkynyl; wherein R^{m} is optionally substituted with 1, 2, 3, 4, 5, 6, 7 or 8 substituents selected from A2;

each of A1 and A2 is independently halo, hydroxy, cyano, nitro, amino, C₁₋₆ alkyl, C₁₋₆ alkoxy, halo C₁₋₆ alkyl or halo C₁₋₆ alkoxy;

each of R¹, R², R³, R⁴ and R⁵ is independently hydrogen, halo, hydroxy, cyano, nitro, amino, carboxy, —C(═O)—NR^{a}R^{b}, —N^{c}—C(═O)—R^{d}, —NR(OR), C₁₋₆ alkyl, C₂₋₈ alkenyl, C₂₋₈ alkynyl, halo C₁₋₆ alkyl, halo C₂₋₈ alkenyl, halo C₂₋₈ alkynyl, hydroxy-substituted C₁₋₆ alkyl, amino-substituted C₁₋₆ alkyl, cyano-substituted C₁₋₆ alkyl, C₁₋₆ alkyl-SO₂—, C₁₋₆ alkyl-(C═O)—, C₁₋₆ alkyl-(C═O)—O—, C₁₋₆ alkoxy, C₁₋₆ alkoxy-(C═O)—, C₁₋₆ alkylthio, C₂₋₈ alkenyloxy, halo C₁₋₆ alkoxy, halo C₁₋₆ alkylthio, halo C₂₋₈ alkenyloxy, hydroxy-substituted C₁₋₆ alkoxy, amino-substituted C₁₋₆ alkoxy, cyano-substituted C₁₋₆ alkoxy or C₁₋₆ alkylamino;

each of R^{a}, R^{b}, R^{c}, R^{d}, R^{e} and R^{f} is independently hydrogen or C₁₋₆ alkyl;

each of R^{A} and R^{B} is independently hydrogen, C₁₋₆ alkyl or halo C₁₋₆ alkyl;

each of R^{C}, R^{C1} and R^{C2} is independently hydrogen, halo or C₁₋₆ alkyl;

each of R⁶, R⁷, R⁸, R⁹, R¹⁰ and R¹¹ is independently hydrogen, C₁₋₄ alkyl, halo C₁₋₄ alkyl, C₁₋₄ alkoxy or halo C₁₋₄ alkoxy;

or Rⁿ and R⁸, together with the atoms to which they are attached, form a 3-8 membered heterocycle; the 3-8 membered heterocycle is optionally substituted with 1, 2, 3, 4, 5, 6, 7 or 8 substituents selected from A3; and

wherein each A3 is independently halo, oxo, hydroxy, cyano, nitro, C₁₋₆ alkyl, C₁₋₆ alkoxy, halo C₁₋₆ alkyl or halo C₁₋₆ alkoxy.

In other embodiments, Rⁿ is C₁₋₄ alkyl, C₂₋₄ alkenyl, C₂₋₄ alkynyl, C₆₋₁₀ aryl or C₆₋₁₀ aryl-C₁₋₃ alkyl-; wherein Rⁿ is optionally substituted with 1, 2, 3, 4, 5, 6, 7 or 8 substituents selected from A1; and

each A1 is independently halo, hydroxy, cyano, nitro, amino, C₁₋₅ alkyl, C₁₋₄ alkoxy, halo C₁₋₄ alkyl or halo C₁₋₄ alkoxy.

In still other embodiments, Rⁿ is —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)CH₃, —C(CH₃)₃, —CH₂F, —CF₂H, —CF₃, —CF₂CHF₂, —CH₂CF₃, —CF₂CHFCF₃, —CH(CF₃)CH₃, —CF(CF₃)₂, —CH₂CH₂—OCH₃ or —CH₂CH₂—OCH₂CH₃;

or Rⁿ is the following sub-structure:

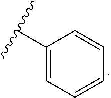 [SMILES: *c1ccccc1]

In some embodiments, R^{m} is C₁₋₄ alkyl, C₂₋₄ alkenyl or C₂₋₄ alkynyl; wherein R^{m} is optionally substituted with 1, 2, 3, 4, 5, 6, 7 or 8 substituents selected from A2; and

each A2 is independently halo, hydroxy, cyano, nitro, amino, C₁₋₄ alkyl, C₁₋₄ alkoxy, halo C₁₋₄ alkyl or halo C₁₋₄ alkoxy.

In other embodiments, R^{m} is —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)CH₃, —C(CH₃)₃, —CH₂F, —CF₂H or —CF₃.

In some embodiments, each of R¹, R², R³, R⁴ and R⁵ is independently hydrogen, halo, hydroxy, cyano, nitro, amino, carboxy, C₁₋₄ alkyl, C₂₋₄ alkenyl, C₂₋₄ alkynyl, halo C₁₋₄ alkyl, halo C₂₋₄ alkenyl, halo C₂₋₄ alkynyl, hydroxy-substituted C₁₋₄ alkyl, amino-substituted C₁₋₄ alkyl, cyano-substituted C₁₋₄ alkyl, C₁₋₄ alkyl-SO₂—, C₁₋₄ alkyl-(C═O)—, C₁₋₄ alkyl-(C═O)—O—, C₁₋₄ alkoxy, C₁₋₄ alkoxy-(C═O)—, C₁₋₄ alkylthio, C₂₋₄ alkenyloxy, halo C₁₋₄ alkoxy, halo C₁₋₄ alkylthio, halo C₂₋₄ alkenyloxy or C₁₋₄ alkylamino.

In other embodiments, each of R¹, R², R³, R⁴ and R⁵ is independently hydrogen, fluoro, chloro, bromo, iodo, hydroxy, cyano, nitro, amino, carboxy, —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)CH₃, —C(CH₃)₃, —CH₂F, —CF₂H, —CF₃, —CF₂CHF₂, —CF₂CHFCF₃, —CF(CF₃)₂, —OCH₃, —OCH₂CH₃, —OCH₂CH₂CH₃, —OCH(CH₃)₂, —OC(CH₃)₃, —OCH₂F, —OCF₂H, —OCF₃, —OCF₂CHF₂, —OCH₂CF₃, —OCF₂CHFCF₃, —OCH(CF₃)CH₃ or —OCF(CF₃)₂.

In some embodiments, each of R^{A} and R^{B} is independently hydrogen, C₁₋₄ alkyl or halo C₁₋₄ alkyl; and

each of R^{C}, R^{C1} and R^{C2} is independently hydrogen, halo or C₁₋₄ alkyl.

In other embodiments, each of R^{A} and R^{B} is independently hydrogen, —CH₃, —CH₂CH₃ or —CHF₂; and

each of R^{C}, R^{C1} and R^{C2} is independently hydrogen, fluoro, chloro, bromo, iodo or —CH₃.

In still other embodiments, each of R⁶, R⁷, R⁸, R⁹, R¹⁰ and R¹¹ is independently hydrogen, C₁₋₂ alkyl, halo C₁₋₂ alkyl.

In some embodiments, Rⁿ and R⁸, together with the atoms to which they are attached, form a 3-6 membered heterocycle; the 3-6 membered heterocycle is optionally substituted with 1, 2, 3, 4, 5, 6, 7 or 8 substituents selected from A3; and

each A3 is independently halo, oxo, hydroxy, cyano, nitro, C₁₋₄ alkyl, C₁₋₄ alkoxy, halo C₁₋₄ alkyl or halo C₁₋₄ alkoxy.

In yet other embodiments, the 3-6 membered heterocycle formed by Rⁿ and R⁸, together with the atoms to which they are attached, is the following sub-structure:

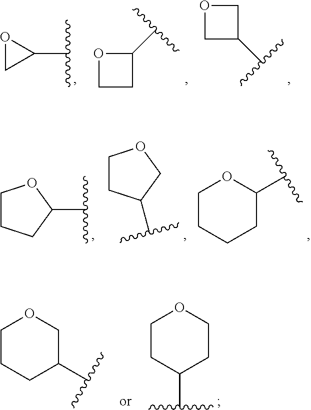

wherein the 3-6 membered heterocycle is optionally substituted with 1, 2, 3, 4, 5, 6, 7 or 8 substituents selected from A3; and

each A3 is independently fluoro, chloro, bromo, iodo, hydroxy, cyano, nitro, —CH₃, —CH₂CH₃, —OCH₃, —OCH₂CH₃, —CF₃ or —OCF₃.

In other aspect, provided herein is a compound of Formula (II) or a stereoisomer, an N-oxide or a salt thereof.

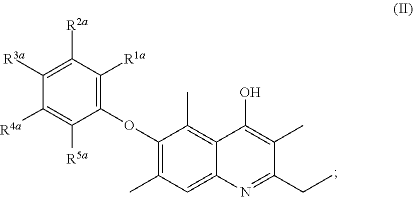

wherein

each of R^{1a}, R^{2a}, R^{3a}, R^{4a} and R^{5a} is independently hydrogen, halo, hydroxy, cyano, nitro, amino, carboxy, C₁₋₄ alkyl, halo C₁₋₄ alkyl, C₁₋₄ alkoxy, C₁₋₄ alkylthio, halo C₁₋₄ alkoxy or halo C₁₋₄ alkylthio;

with the proviso that the compound of Formula (II) is not 2-ethyl-3,5,7-trimethyl-6-(4-(1,1,2,2-tetrafluoroethoxy)phenoxy)quinolin-4-ol.

In some embodiments, each of R^{1a}, R^{2a}, R^{3a}, R^{4a} and R^{5a} is independently hydrogen, fluoro, chloro, bromo, iodo, hydroxy, cyano, nitro, amino, carboxy, —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)CH₃, —C(CH₃)₃, —CH₂F, —CF₂H, —CF₃, —CF₂CHF₂, —CF₂CHFCF₃, —CF(CF₃)₂, —OCH₃, —OCH₂CH₃, —OCH₂CH₂CH₃, —OCH(CH₃)₂, —OC(CH₃)₃, —OCH₂F, —OCF₂H, —OCF₃, —OCH₂CF₃, —OCF₂CHF₂, —OCF₂CHFCF₃, —OCH(CF₃)CH₃ or —OCF(CF₃)₂;

with the proviso that when R^{1a}, R^{2a}, R^{4a} and R^{5a} are hydrogen, R^{3a} is not —OCF₂CHF₂.

In other aspect, provided herein is a composition containing the compound of the invention, wherein the composition further comprises an agriculturally acceptable surfactant and/or carrier.

In other aspect, provided herein is use of the compound or the composition for controlling pests.

### DETAILED DESCRIPTION OF THE INVENTION

### Definitions and General Terminology

Reference will now be made in detail to certain embodiments of the invention, examples of which are illustrated in the accompanying structures and formulas. The invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described herein. In the event that one or more of the incorporated literature, patents, and similar materials differs from or contradicts this application, including but not limited to defined terms, term usage, described techniques, or the like, this application controls.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Unless defined otherwise, all technical and scientific terms used herein have the same meaning as is commonly understood by one skilled in the art to which this invention belongs. All patents and publications referred to herein are incorporated by reference in their entirety.

As used herein, the following definitions shall apply unless otherwise indicated. For purposes of this invention, the chemical elements are identified in accordance with the Periodic Table of the Elements, CAS version, and the Handbook of Chemistry and Physics, 75th Ed. 1994. Additionally, general principles of organic chemistry are described in “Organic Chemistry”, Thomas Sorrell, University Science Books, Sausalito: 1999, and Smith et al., “March's Advanced Organic Chemistry”, John Wiley & Sons, New York: 2007, the entire contents of which are hereby incorporated by reference.

The grammatical articles “a”, “an” and “the”, as used herein, are intended to include “at least one” or “one or more” unless otherwise indicated herein or clearly contradicted by the context. Thus, the articles are used herein to refer to one or more than one (i.e. at least one) of the grammatical objects of the article. By way of example, “a component” means one or more components, and thus, possibly, more than one component is contemplated and may be employed or used in an implementation of the described embodiments.

The term “comprise” is an open expression, it means comprising the contents disclosed herein, but don't exclude other contents.

“Stereoisomers” refers to compounds which have identical chemical constitution, but differ with regard to the arrangement of the atoms or groups in space. Stereoisomers include enantiomer, diastereomers, conformer (rotamer), geometric (cis/trans) isomer, atropisomer, etc.

“Enantiomers” refers to two stereoisomers of a compound which are non-superimposable mirror images of one another.

“Diastereomer” refers to stereoisomers with two or more centers of chirality and whose molecules are not mirror images of one another. Diastereomers have different physical properties, e.g. melting points, boiling points, spectral properties or biological activities. Mixture of diastereomers may separate under high resolution analytical procedures such as electrophoresis and chromatography such as HPLC.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994.

Many organic compounds exist in optically active forms, i.e., they have the ability to rotate the plane of plane-polarized light. When describing an optically active compound, the prefixes D and L, or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. A specific stereoisomer may be referred to as an enantiomer, and a mixture of such stereoisomers is called an enantiomeric mixture. A 50:50 mixture of enantiomers is referred to as a racemic mixture or a racemate, which may occur where there has been no stereoselection or stereospecificity in a chemical reaction or process.

Any asymmetric atom (e.g., carbon or the like) of the compound(s) disclosed herein can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration.

Depending on the choice of the starting materials and procedures, the compounds can be present in the form of one of the possible stereoisomers or as mixtures thereof, such as racemates and diastereoisomer mixtures, depending on the number of asymmetric carbon atoms. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, substituents of the cycloalkyl may have a cis- or trans-configuration.

Any resulting mixtures of stereoisomers can be separated on the basis of the physicochemical differences of the constituents, into the pure or substantially pure geometric isomers, enantiomers, diastereomers, for example, by chromatography and/or fractional crystallization. Cis and trans isomers are diastereomer.

Any resulting racemates of final products or intermediates can be resolved into the optical antipodes by methods known to those skilled in the art, e.g., by separation of the diastereomeric salts thereof. Racemic products can also be resolved by chiral chromatography, e.g., high performance liquid chromatography (HPLC) using a chiral adsorbent. In particular, enantiomers can be prepared by asymmetric synthesis.

As described herein, compounds disclosed herein may optionally be substituted with one or more substituents, such as are illustrated generally below, or as exemplified by particular classes, subclasses, and species of the invention. It will be appreciated that the phrase “optionally substituted” is used interchangeably with the phrase “substituted or unsubstituted”. In general, the term “substituted” refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. Specifically, “one or more” before an example refers to 1, 2, 3, 4, 5, 6, 7, 8, 9 or 10. wherein substituents of compounds disclosed herein include, but are not limited to, deuterium, fluoro (F), chloro (Cl), bromo (Br), iodo (I), cyano (CN), hydroxy (OH), nitro (NO₂), amino (NH₂), carboxy (COOH), alkyl, alkoxy, alkoxyalkyl, alkoxyalkoxy, alkoxyalkylamino, aryloxy, heteroaryloxy, heterocyclyloxy, arylalkoxy, heteroarylalkoxy, heterocyclylalkoxy, cycloalkylalkoxy, alkylamino, alkylaminoalkyl, alkylaminoalkylamino, cycloalkylamino, cycloalkylalkylamino, alkylthio, haloalkyl, haloalkoxy, hydroxy-substituted alkyl, hydroxy-substituted alkylamino, cyano-substituted alkyl, cyano-substituted alkoxy, cyano-substituted alkylamino, amino-substituted alkyl, alkylacyl, heteroalkyl, cycloalkyl, cycloalkenyl, cycloalkylalkyl, heterocyclyl, heterocyclylalkyl, heterocyclylacyl, aryl, arylalkyl, arylamino, heteroaryl, heteroarylalkyl, heteroarylamino, acylamino, sulfonyl, aminosulfonyl, and the like.

Furthermore, what needs to be explained is that the phrases “each . . . is independently” and “each of . . . and . . . is independently”, unless otherwise stated, should be broadly understood. The specific options expressed by the same symbol are independent of each other in different groups; or the specific options expressed by the same symbol are independent of each other in same groups.

At various places in the present specification, substituents of compounds disclosed herein are disclosed in groups or in ranges. It is specifically intended that the invention include each and every individual subcombination of the members of such groups and ranges. For example, the term “C₁-C₆ alkyl” or “C₁₋₆ alkyl” is specifically intended to individually disclose methyl, ethyl, C₃ alkyl, C₄ alkyl, C₅ alkyl, and C₆ alkyl.

The term “alkyl” or “alkyl group” refers to a saturated linear or branched-chain monovalent hydrocarbon group of 1-20 carbon atoms, wherein the alkyl group is optionally substituted with one or more substituents described herein. Unless otherwise stated, the alkyl group contains 1-20 carbon atoms. In some embodiments, the alkyl group contains 1-12 carbon atoms. In some embodiments, the alkyl group contains 1-8 carbon atoms. In other embodiments, the alkyl group contains 1-6 carbon atoms. In still other embodiments, the alkyl group contains 1-4 carbon atoms. In yet other embodiments, the alkyl group contains 1-3 carbon atoms.

Some non-limiting examples of the alkyl group include, methyl (Me, —CH₃), ethyl (Et, —CH₂CH₃), n-propyl (n-Pr, —CH₂CH₂CH₃), isopropyl (i-Pr, —CH(CH₃)₂), n-butyl (n-Bu, —CH₂CH₂CH₂CH₃), isobutyl (i-Bu, —CH₂CH(CH₃)₂), sec-butyl (s-Bu, —CH(CH₃)CH₂CH₃), tert-butyl (t-Bu, —C(CH₃)₃), n-pentyl (—CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), n-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, etc.

The term “alkenyl” refers to linear or branched-chain monovalent hydrocarbon radical of 2 to 12 carbon atoms with at least one site of unsaturation, i.e., a carbon-carbon, sp² double bond, wherein the alkenyl radical may be optionally substituted independently with one or more substituents described herein, and includes radicals having “cis” and “trans” orientations, or alternatively, “E” and “Z” orientations. In some embodiments, the alkenyl contains 2 to 8 carbon atoms. In other embodiments, the alkenyl contains 2 to 6 carbon atoms. In still other embodiments, the alkenyl contains 2 to 4 carbon atoms. Some non-limiting examples of the alkenyl group include ethenyl or vinyl (—CH═CH₂), allyl (—CH₂CH═CH₂), propenyl (CH₃—CH═CH—), oxo butenyl (CH₃—C(═O)—CH═CH—) and the like.

The term “alkynyl” refers to a linear or branched monovalent hydrocarbon radical of 2 to 12 carbon atoms with at least one carbon-carbon, sp triple bond. In some embodiments, the alkynyl contains 2 to 8 carbon atoms. In other embodiments, the alkynyl contains 2 to 6 carbon atoms. In still other embodiments, the alkynyl contains 2 to 4 carbon atoms. Some non-limiting examples of the alkynyl group include —C≡CH, —CH₂—C≡CH, —CH₂—C≡CCH₃, —CH₂CH₂—C≡CH, —CH₂—C≡CCH₂CH₃, and the like.

The term “alkoxy” refers to an alkyl group, as previously defined, attached to the parent molecular moiety via an oxygen atom. Some non-limiting examples of the alkoxy group include methoxy (MeO, —OCH₃), ethoxy (EtO, —OCH₂CH₃), 1-propoxy (n-PrO, n-propoxy, —OCH₂CH₂CH₃), 2-propoxy (i-PrO, i-propoxy, —OCH(CH₃)₂) and the like.

The term “alkylthio” refers to a radical containing a linear or branched-alkyl radical, attached to a divalent sulfur atom. Wherein the alkyl group is as defined herein. Examples of the alkylthio group include, but are not limited to, —SCH₃, —SCH₂CH₃, —SCH₂CH₂CH₃, and the like.

The term “halogen” or “halo” refers to fluorine (fluoro, F), chlorine (chloro, Cl), bromine (bromo, Br) or iodine (iodo, I).

The term “oxo” refers to —CH₂— replaced by —C(═O)—.

The term “carboxy” refers to —COOH.

The term “haloalkyl” refers to an alkyl group substituted with one or more halogen atoms. Some non-limiting examples of such groups include, —CF₃, —CHF₂, —CH₂Cl, —CH₂CF₃, —CH₂CHF₂, —CH₂CH₂CF₃ and the like.

The term “haloalkoxy” refers to an alkoxy group substituted with one or more halogen atoms. Some non-limiting examples of such groups include, —OCF₃, —OCHF₂, —OCHCl₂, —OCH₂CHF₂, —OCH₂CHCl₂, —OCH(CH₃)CHF₂, and the like.

The term “haloalkylthio” refers to an alkylthio group substituted with one or more halogen atoms. Some non-limiting examples of such groups include, —SCF₃, —SCHF₂, —SCHC₂, —SCH₂CHF₂, —SCH₂CHCl₂, —SCH(CH₃)CHF₂, and the like.

The term “haloalkenyl” refers to an alkenyl group substituted with one or more halogen atoms.

The term “haloalkynyl” refers to an alkynyl group, substituted with one or more halogen atoms.

The term “aryl” refers to monocyclic, bicyclic and tricyclic carbocyclic ring systems having a total of six to fourteen ring members, or six to twelve ring members, or six to ten ring members, wherein at least one ring in the system is aromatic, wherein each ring in the system contains 3 to 7 ring members and that has a single point or multipoint of attachment to the rest of the molecule. The term “aryl” and “aromatic ring” can be used interchangeably herein. Examples of the aryl group may include phenyl, indenyl, naphthyl, and anthryl.

The term “arylalkyl” refers to an alkyl group in which hydrogen atoms are substituted with one or more aryl groups, wherein the alkyl and aryl groups are as defined herein. Some non-limiting examples of such group include benzyl, phenylethyl, and the like.

The term “heterocycle”, “heterocyclyl”, or “heterocyclic ring” as used interchangeably herein refers to a saturated or partially unsaturated monocyclic, bicyclic or tricyclic ring containing 3-15 ring atoms, wherein the monocyclic, bicyclic or tricyclic ring does not contain aromatic ring, of which at least one ring atom is selected from nitrogen, sulfur or oxygen. Unless otherwise specified, the heterocyclyl group may be carbon or nitrogen linked, and a —CH₂— group can be optionally replaced by a —C(═O)— group. In which, the sulfur on the ring can be optionally oxygenized to S-oxide and the nitrogen on the ring can be optionally oxygenized to N-oxide. Examples of heterocyclyl include, but are not limited to, oxiranyl, azetidinyl, oxetanyl, thietanyl, pyrrolidinyl (such as 2-pyrrolidinyl), 2-pyrrolinyl, 3-pyrrolinyl, pyrazolidinyl, imidazolinyl, imidazolidinyl, tetrahydrofuranyl, dihydrofuranyl, tetrahydrothienyl, dihydrothienyl, 1,3-dioxolanyl, dithiolanyl, tetrahydropyranyl, dihydropyranyl, 2H-pyranyl, 4H-pyranyl, tetrahydrothiopyranyl, piperidinyl (such as 2-piperidinyl, 3-piperidinyl, 4-piperidinyl), morpholinyl, thiomorpholinyl, 1-oxidothiomorpholinyl, 1,1-dioxidothiomorpholinyl, piperazinyl, dioxanyl, dithianyl, thioxanyl, homopiperazinyl, homopiperidinyl, oxepanyl, thiepanyl, 2-oxa-5-azabicyclo[2.2.1]hept-5-yl, tetrahydropyridyl. Some non-limiting examples of heterocyclyl wherein —CH₂— group is replaced by —C(═O)— moiety include 2-oxopyrrolidinyl, oxo-1,3-thiazolidinyl, 2-piperidinonyl and 3,5-dioxopiperidinyl. Some non-limited examples of heterocyclyl wherein the ring sulfur atom is oxidized is sulfolanyl, 1,1-dioxo-thiomorpholinyl; and wherein the heterocyclyl group is optionally substituted with one or more substituents described herein.

The terms “3-12 membered heterocyclyl”, “3-10 membered heterocyclyl”, “3-8 membered heterocyclyl” or “3-6 membered heterocyclyl”, wherein “3-12 membered” “3-10 membered” “3-8 membered” or “3-6 membered” typically described ring atoms number of a molecule. For example, piperidinyl is 6-membered heterocyclyl.

The term “heteroatom” refers to oxygen, sulfur, nitrogen, phosphorus and silicon, including any oxidized form of nitrogen, sulfur, or phosphorus; forms of primary, secondary, tertiary amines and quaternary ammonium salts; or a substitutable nitrogen of a heterocyclic ring, for example, N (such as N of 3,4-dihydro-2H-pyrrolyl), NH (such as NH of pyrrolidinyl) or NR (such as NR of N-substituted pyrrolidinyl).

When the compound of the invention contains an acid part, the salt of the compound of the invention includes those derived from alkali metals or alkaline earth metals and those derived from ammonia and amine. Preferred cations include sodium, potassium, magnesium and ammonium cations having the chemical formula N⁺(R¹⁹R²⁰R²¹R²²), wherein each R¹⁹, R²⁰, R²¹ and R²² is independently selected from C₁-C₆ alkyl and C₁-C₆ hydroxyalkyl. Salts of the compounds of formula (I) or formula (II) can be prepared by treating compounds of formula (I) or formula (II) with metal hydroxides (such as sodium hydroxide) or amines (such as ammonia, trimethylamine, diethanolamine, 2-methylthiopropylamine, diallylamine, 2-butoxyethylamine, morpholine, cyclic dodecylamine or benzylamine).

When the compound of the present invention contains an alkali part, acceptable salts can be formed from organic and inorganic acids, such as acetic acid, propionic acid, lactic acid, citric acid, tartaric acid, succinic acid, fumaric acid, maleic acid, malonic acid, mandelic acid, malic acid, phthalic acid, hydrochloric acid, hydrobromic acid, phosphoric acid, nitric acid, sulfuric acid, methylsulfonic acid, naphthalene sulfonic acid, benzenesulfonic acid, toluene sulfonic acid, camphor sulfonic acid and similar known acceptable acids.

### Detailed Description of Compounds of the Invention

The purpose of the present invention is to provide a compound having significant effects on pests control, pesticidal compositions and formulations both containing the compound and uses thereof.

In one aspect, provided herein is a compound of Formula (I) or a stereoisomer, an N-oxide or an acceptable salt thereof:

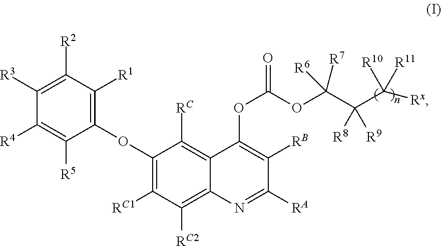

wherein

R^{x} is

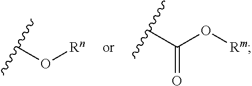

Rⁿ is alkyl, alkenyl, alkynyl, aryl or arylalkyl; wherein Rⁿ is optionally substituted with 1, 2, 3, 4, 5, 6, 7 or 8 substituents selected from A1;

R^{m} is alkyl, alkenyl or alkynyl; wherein R^{m} is optionally substituted with 1, 2, 3, 4, 5, 6, 7 or 8 substituents selected from A2;

each of A1 and A2 is independently halo, hydroxy, cyano, nitro, amino, alkyl, alkoxy, haloalkyl or haloalkoxy;

each of R¹, R², R³, R⁴ and R⁵ is independently hydrogen, halo, hydroxy, cyano, nitro, amino, carboxy, —C(═O)—NR^{a}R^{b}, —R^{c}—C(═O)—R^{d}, —NR(OR), alkyl, alkenyl, alkynyl, haloalkyl, haloalkenyl, haloalkynyl, hydroxy-substituted alkyl, amino-substituted alkyl, cyano-substituted alkyl, alkyl-SO₂—, alkyl-(C═O)—, alkyl-(C═O)—O—, alkoxy, alkoxy-(C═O)—, alkylthio, alkenyloxy, haloalkoxy, haloalkylthio, haloalkenyloxy, hydroxy-substituted alkoxy, amino-substituted alkoxy, cyano-substituted alkoxy or alkylamino;

each of R^{a}, R^{b}, R^{c}, R^{d}, R^{e} and R^{f} is independently hydrogen or alkyl;

or R¹ and R², or R² and R³, or R³ and R⁴, or R⁴ and R⁵ may form —O—(CH₂)ₘ—O—, —(CH₂)ₘ₁—O— or —(CH₂)ₘ₂—, wherein each of —O—(CH₂)ₘ—O—, —(CH₂)ₘ₁—O— and —(CH₂)ₘ₂— is independently optionally substituted with 1, 2, 3, 4, 5 or 6 halo;

wherein each of m, m1 and m2 is independently 1, 2 or 3;

each of R^{A} and R^{B} is independently hydrogen, alkyl or haloalkyl;

each of R^{C}, R^{C1} and R^{C2} is independently hydrogen, halo or alkyl;

each of R⁶, R⁷, R⁸, R⁹, R¹⁰ and R¹¹ is independently hydrogen, alkyl, haloalkyl, alkoxy or haloalkoxy;

or Rⁿ and R⁸, together with the atoms to which they are attached, form a 3-8 membered heterocycle, the 3-8 membered heterocycle is optionally substituted with 1, 2, 3, 4, 5, 6, 7 or 8 substituents selected from A3;

wherein each A3 is independently halo, oxo, hydroxy, cyano, nitro, alkyl, alkoxy, haloalkyl or haloalkoxy; and

n is 0, 1, 2 or 3.

In some embodiments, Rⁿ is C₁₋₆ alkyl, C₂₋₈ alkenyl, C₂₋₈ alkynyl, C₆₋₁₄ aryl or C₆₋₁₄ aryl-C₁₋₆ alkyl-; wherein Rⁿ is optionally substituted with 1, 2, 3, 4, 5, 6, 7 or 8 substituents selected from A1; and

each A1 is independently halo, hydroxy, cyano, nitro, amino, C₁₋₆ alkyl, C₁₋₆ alkoxy, halo C₁₋₆ alkyl or halo C₁₋₆ alkoxy.

In some embodiments, R^{m} is C₁₋₆ alkyl, C₂₋₈ alkenyl or C₂₋₈ alkynyl; wherein R^{m} is optionally substituted with 1, 2, 3, 4, 5, 6, 7 or 8 substituents selected from A2;

each A2 is independently halo, hydroxy, cyano, nitro, amino, C₁₋₆ alkyl, C₁₋₆ alkoxy, halo C₁₋₆ alkyl or halo C₁₋₆ alkoxy.

In some embodiments, each of R¹, R², R³, R⁴ and R⁵ is independently hydrogen, halo, hydroxy, cyano, nitro, amino, carboxy, —C(═O)—NR^{a}R^{b}, —R^{c}—C(═O)—R^{d}, —NR(OR), C₁₋₆ alkyl, C₂₋₈ alkenyl, C₂₋₈ alkynyl, halo C₁₋₆ alkyl, halo C₂₋₈ alkenyl, halo C₂₋₈ alkynyl, hydroxy-substituted C₁₋₆ alkyl, amino-substituted C₁₋₆ alkyl, cyano-substituted C₁₋₆ alkyl, C₁₋₆ alkyl-SO₂—, C₁₋₆ alkyl-(C═O)—, C₁₋₆ alkyl-(C═O)—O—, C₁₋₆ alkoxy, C₁₋₆ alkoxy-(C═O)—, C₁₋₆ alkylthio, C₂₋₈ alkenyloxy, halo C₁₋₆ alkoxy, halo C₁₋₆ alkylthio, halo C₂₋₈ alkenyloxy, hydroxy-substituted C₁₋₆ alkoxy, amino-substituted C₁₋₆ alkoxy, cyano-substituted C₁₋₆ alkoxy or C₁₋₆ alkylamino; and

each of R^{a}, R^{b}, R^{c}, R^{d}, R^{e} and R^{f} is independently hydrogen or C₁₋₆ alkyl.

In some embodiments, each of R^{A} and R^{B} is independently hydrogen, C₁₋₆ alkyl or halo C₁₋₆ alkyl.

In some embodiments, each of R^{C}, R^{C1} and R^{C2} is independently hydrogen, halo or C₁₋₆ alkyl.

In some embodiments, each of R⁶, R⁷, R⁸, R⁹, R¹⁰ and R¹¹ is independently hydrogen, C₁₋₄ alkyl, halo C₁₋₄ alkyl, C₁₋₄ alkoxy or halo C₁₋₄ alkoxy.

In other embodiments, Rⁿ is C₁₋₄ alkyl, C₂₋₄ alkenyl, C₂₋₄ alkynyl, C₆₋₁₀ aryl or C₆₋₁₀ aryl-C₁₋₃ alkyl-; wherein Rⁿ is optionally substituted with 1, 2, 3, 4, 5, 6, 7 or 8 substituents selected from A1; and

each A1 is independently halo, hydroxy, cyano, nitro, amino, C₁₋₅ alkyl, C₁₋₄ alkoxy, halo C₁₋₄ alkyl or halo C₁₋₄ alkoxy.

In still other embodiments, Rⁿ [symbol] -CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)CH₃, —C(CH₃)₃, —CH₂F, —CF₂H, —CF₃, —CF₂CHF₂, —CH₂CF₃, —CF₂CHFCF₃, —CH(CF₃)CH₃, —CF(CF₃)₂, —CH₂CH₂—OCH₃ or —CH₂CH₂—OCH₂CH₃.

In yet other embodiments, Rⁿ is the following sub-structure:

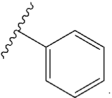 [SMILES: *c1ccccc1]

In some embodiments, R^{m} is C₁₋₄ alkyl, C₂₋₄ alkenyl or C₂₋₄ alkynyl; wherein R^{m} is optionally substituted with 1, 2, 3, 4, 5, 6, 7 or 8 substituents selected from A2; and

each A2 is independently halo, hydroxy, cyano, nitro, amino, C₁₋₄ alkyl, C₁₋₄ alkoxy, halo C₁₋₄ alkyl or halo C₁₋₄ alkoxy.

In other embodiments, R^{m} is —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)CH₃, —C(CH₃)₃, —CH₂F, —CF₂H or —CF₃.

In some embodiments, each of R¹, R², R³, R⁴ and R⁵ is independently hydrogen, halo, hydroxy, cyano, nitro, amino, carboxy, C₁₋₄ alkyl, C₂₋₄ alkenyl, C₂₋₄ alkynyl, halo C₁₋₄ alkyl, halo C₂₋₄ alkenyl, halo C₂₋₄ alkynyl, hydroxy-substituted C₁₋₄ alkyl, amino-substituted C₁₋₄ alkyl, cyano-substituted C₁₋₄ alkyl, C₁₋₄ alkyl-SO₂—, C₁₋₄ alkyl-(C═O)—, C₁₋₄ alkyl-(C═O)—O—, C₁₋₄ alkoxy, C₁₋₄ alkoxy-(C═O)—, C₁₋₄ alkylthio, C₂₋₄ alkenyloxy, halo C₁₋₄ alkoxy, halo C₁₋₄ alkylthio, halo C₂₋₄ alkenyloxy or C₁₋₄ alkylamino.

In other embodiments, each of R¹, R², R³, R⁴ and R⁵ is independently hydrogen, fluoro, chloro, bromo, iodo, hydroxy, cyano, nitro, amino, carboxy, —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)CH₃, —C(CH₃)₃, —CH₂F, —CF₂H, —CF₃, —CF₂CHF₂, —CF₂CHFCF₃, —CF(CF₃)₂, —OCH₃, —OCH₂CH₃, —OCH₂CH₂CH₃, —OCH(CH₃)₂, —OC(CH₃)₃, —OCH₂F, —OCF₂H, —OCF₃, —OCF₂CHF₂, —OCH₂CF₃, —OCF₂CHFCF₃, —OCH(CF₃)CH₃ or —OCF(CF₃)₂.

In some embodiments, each of R^{A} and R^{B} is independently hydrogen, C₁₋₄ alkyl or halo C₁₋₄ alkyl.

In other embodiments, each of R^{A} and R^{B} is independently hydrogen, —CH₃, —CH₂CH₃ or —CHF₂.

In some embodiments, each of R^{C}, R^{C1} and R^{C2} is independently hydrogen, halo or C₁₋₄ alkyl.

In other embodiments, each of R^{C}, R^{C1} and R^{C2} is independently hydrogen, fluoro, chloro, bromo, iodo or —CH₃.

Instill other embodiments, each of R⁶, R⁷, R⁸, R⁹, R¹⁰ and R¹¹ is independently hydrogen, C₁₋₂ alkyl or halo C₁₋₂ alkyl.

In some embodiments, Rⁿ and R⁸, together with the atoms to which they are attached, form a 3-6 membered heterocycle; the 3-6 membered heterocycle is optionally substituted with 1, 2, 3, 4, 5, 6, 7 or 8 substituents selected from A3; and

each A3 is independently halo, oxo, hydroxy, cyano, nitro, C₁₋₄ alkyl, C₁₋₄ alkoxy, halo C₁₋₄ alkyl or halo C₁₋₄ alkoxy.

In yet other embodiments, the 3-6 membered heterocycle formed by Rⁿ and R⁸, together with the atoms to which they are attached, is the following sub-structure:

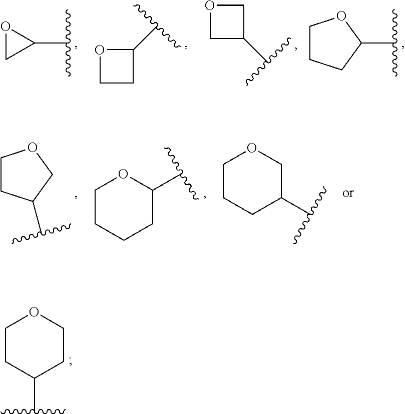

wherein the 3-6 membered heterocycle is optionally substituted with 1, 2, 3, 4, 5, 6, 7 or 8 substituents selected from A3; and

each A3 is independently fluoro, chloro, bromo, iodo, hydroxy, cyano, nitro, —CH₃, —CH₂CH₃, —OCH₃, —OCH₂CH₃, —CF₃ or —OCF₃.

In some embodiments, provided herein is a compound having one of the following structures or a stereoisomer, an N-oxide or a salt thereof:

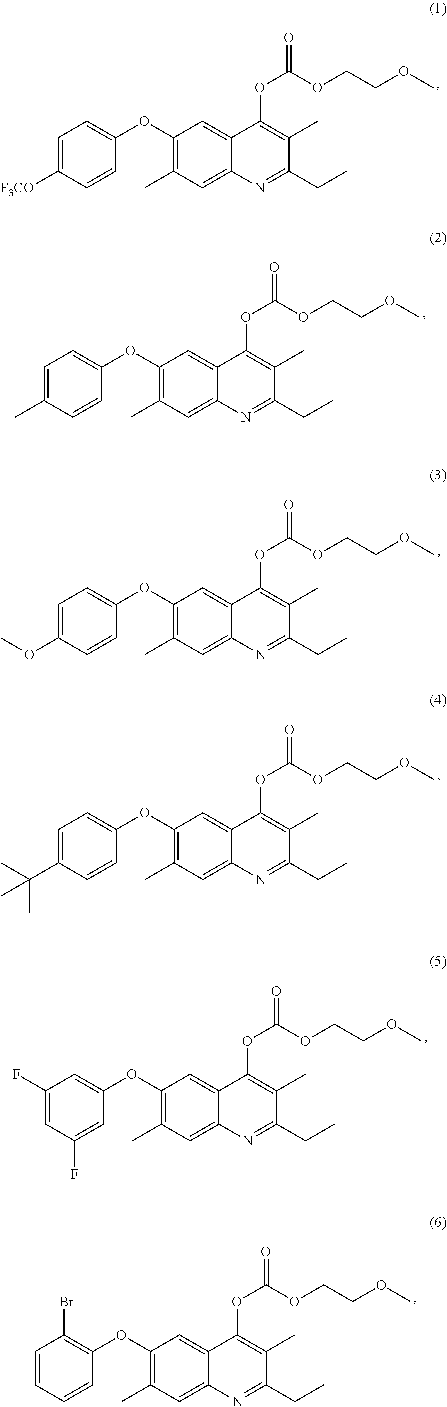

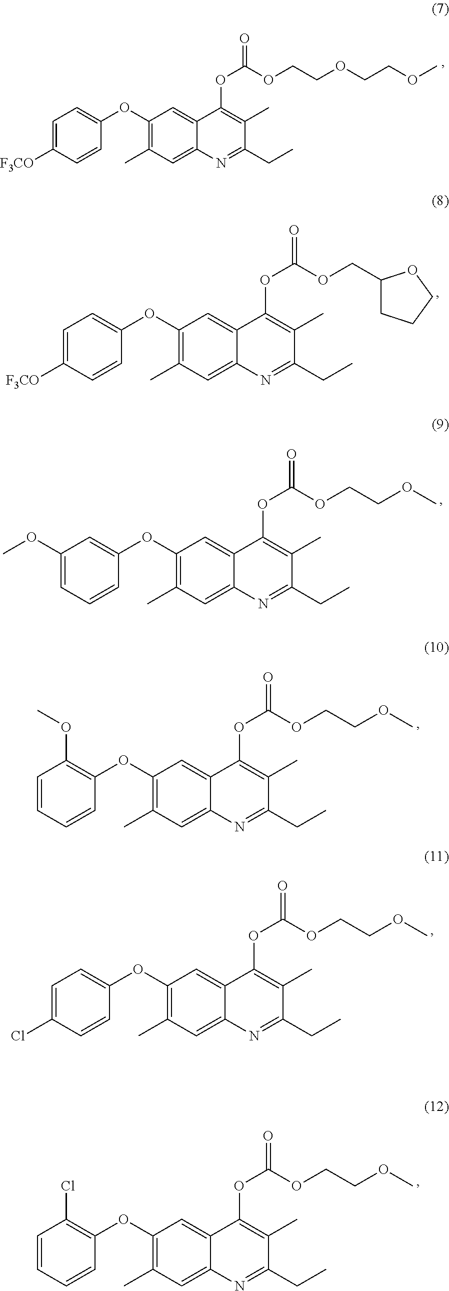

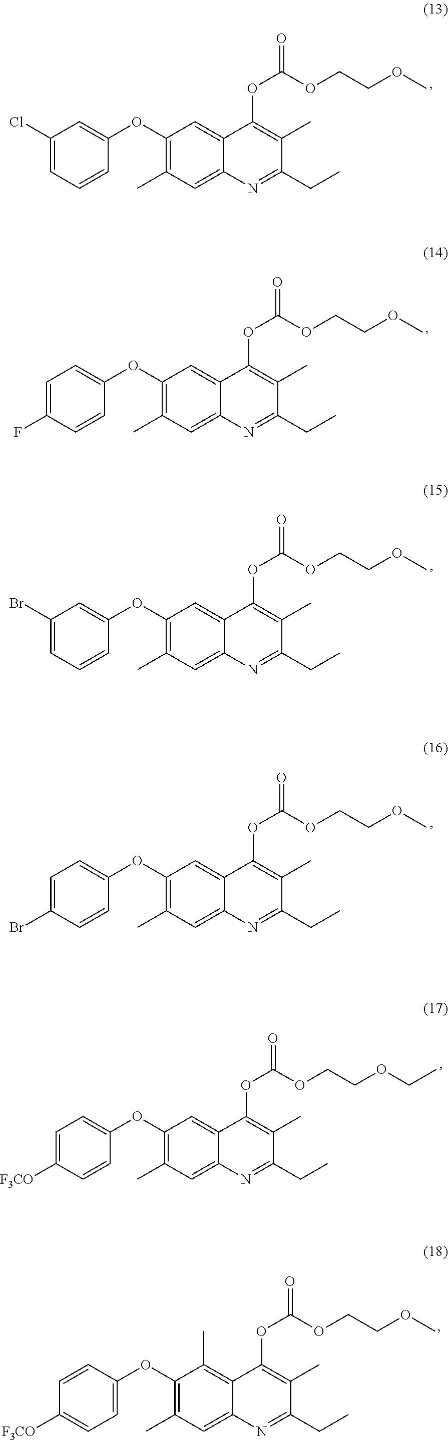 [SMILES: CCc1nc2cc(C)c(Oc3cccc(Cl)c3)cc2c(OC(=O)OCCOC)c1C ; CCc1nc2cc(C)c(Oc3ccc(F)cc3)cc2c(OC(=O)OCCOC)c1C ; CCc1nc2cc(C)c(Oc3cccc(Br)c3)cc2c(OC(=O)OCCOC)c1C ; CCc1nc2cc(C)c(Oc3ccc(Br)cc3)cc2c(OC(=O)OCCOC)c1C ; CCOCCOC(=O)Oc1c(C)c(CC)nc2cc(C)c(Oc3ccc(OC(F)(F)F)cc3)cc12 ; CCc1nc2cc(C)c(Oc3ccc(OC(F)(F)F)cc3)c(C)c2c(OC(=O)OCCOC)c1C]

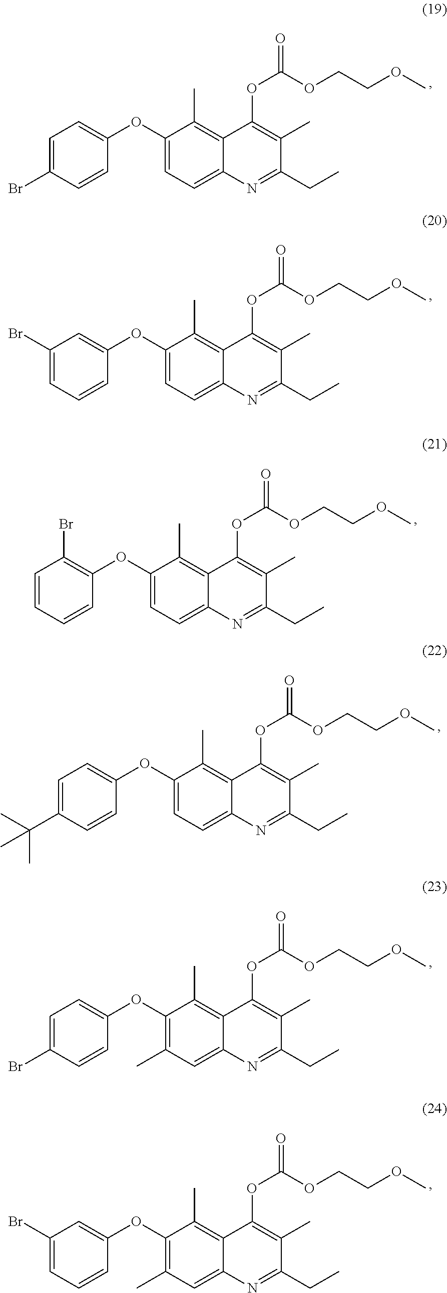

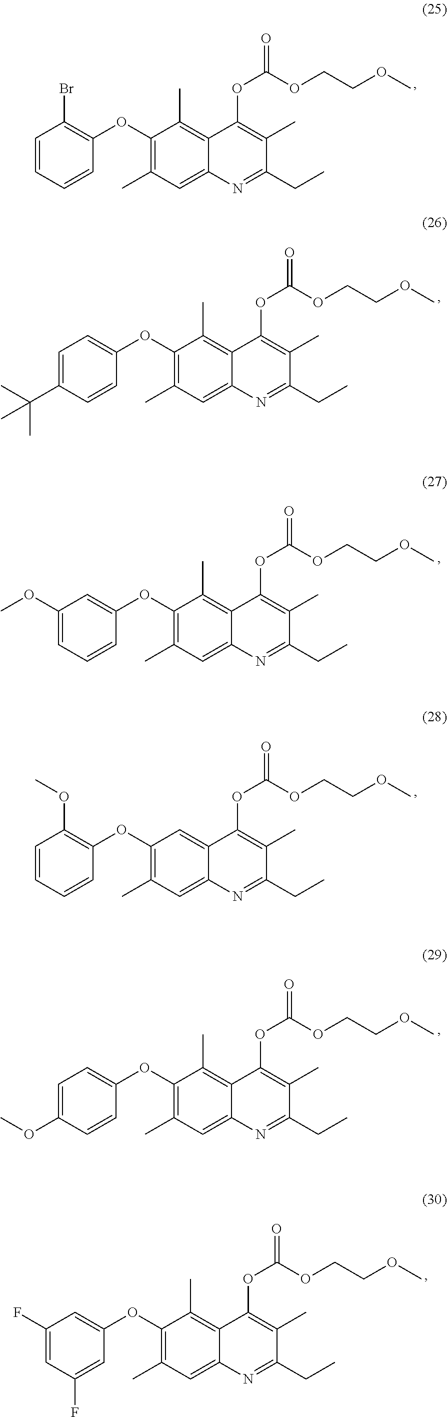

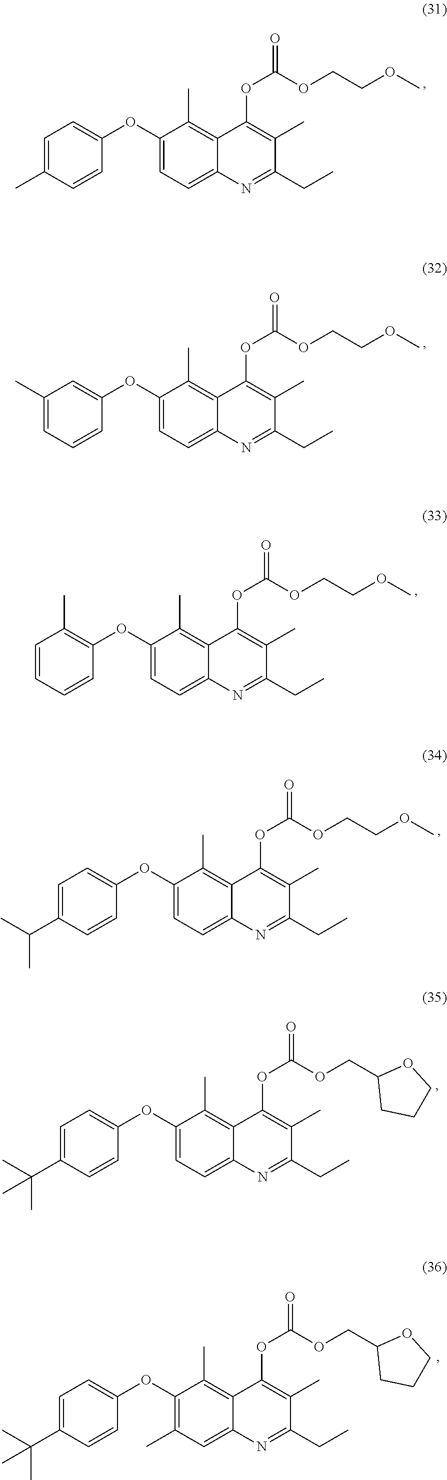

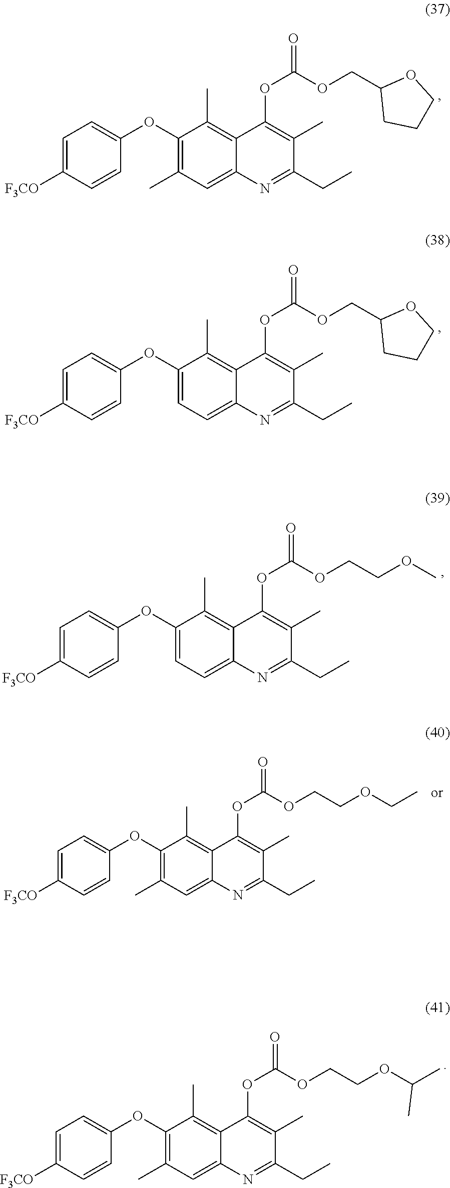 [SMILES: CCc1nc2cc(C)c(Oc3ccc(OC(F)(F)F)cc3)c(C)c2c(OC(=O)OCC2CCCO2)c1C ; CCc1nc2ccc(Oc3ccc(OC(F)(F)F)cc3)c(C)c2c(OC(=O)OCC2CCCO2)c1C ; CCc1nc2ccc(Oc3ccc(OC(F)(F)F)cc3)c(C)c2c(OC(=O)OCCOC)c1C ; CCOCCOC(=O)Oc1c(C)c(CC)nc2cc(C)c(Oc3ccc(OC(F)(F)F)cc3)c(C)c12 ; CCc1nc2cc(C)c(Oc3ccc(OC(F)(F)F)cc3)c(C)c2c(OC(=O)OCCOC(C)C)c1C]

In other aspect, provided herein is a compound of Formula (II) or a stereoisomer, an N-oxide or a salt thereof:

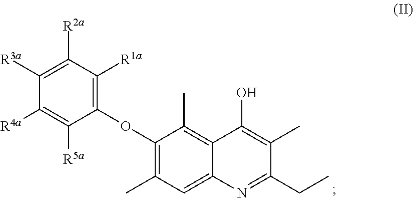

wherein

each of R^{1a}, R^{2a}, R^{3a}, R^{4a} and R^{5a} is independently hydrogen, halo, hydroxy, cyano, nitro, amino, carboxy, C₁₋₄ alkyl, halo C₁₋₄ alkyl, C₁₋₄ alkoxy, C₁₋₄ alkylthio, halo C₁₋₄ alkoxy or halo C₁₋₄ alkylthio;

with the proviso that the compound of Formula (II) is not 2-ethyl-3,5,7-trimethyl-6-(4-(1,1,2,2-tetrafluoroethoxy)phenoxy)quinolin-4-ol; wherein

2-ethyl-3,5,7-trimethyl-6-(4-(1,1,2,2-tetrafluoroethoxy)phenoxy)quinolin-4-ol has the following structure:

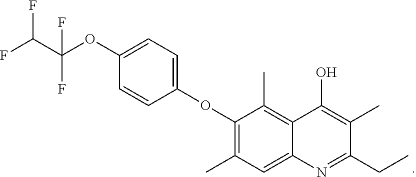 [SMILES: CCc1nc2cc(C)c(Oc3ccc(OC(F)(F)C(F)F)cc3)c(C)c2c(O)c1C]

In some embodiments, each of R^{1a}, R^{2a}, R^{3a}, R^{4a} and R^{5a} is independently hydrogen, fluoro, chloro, bromo, iodo, hydroxy, cyano, nitro, amino, carboxy, —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)CH₃, —C(CH₃)₃, —CH₂F, —CF₂H, —CF₃, —CF₂CHF₂, —CF₂CHFCF₃, —CF(CF₃)₂, —OCH₃, —OCH₂CH₃, —OCH₂CH₂CH₃, —OCH(CH₃)₂, —OC(CH₃)₃, —OCH₂F, —OCF₂H, —OCF₃, —OCH₂CF₃, —OCF₂CHF₂, —OCF₂CHFCF₃, —OCH(CF₃)CH₃ or —OCF(CF₃)₂;

with the proviso that when R^{1a}, R^{2a}, R^{4a} and R^{5a} are hydrogen, R^{3a} is not —OCF₂CHF₂.

In some embodiments, provided herein is a compound having the following structure or a stereoisomer, an N-oxide or a salt thereof:

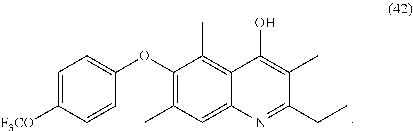 [SMILES: CCc1nc2cc(C)c(Oc3ccc(OC(F)(F)F)cc3)c(C)c2c(O)c1C]

In other aspect, provided herein is a composition containing the compound of the invention, wherein the composition further comprises an agriculturally acceptable surfactant and/or carrier.

In other aspect, the invention provides use of the compound or the composition described herein for controlling pests.

Further, the pests of the invention include Diamondback moth (Plutella xylostella), Tobacco Cutworm (Prodenia litura), Beet Armyworm (, Spodoptera exigua Hü bner), Armyworm (Mythimna separata), Frankliniella occidentalis (Pergande), Brown rice planthopper (Nilaparvata lugens), Carmine Spider Mite (Tetranychus cinnabarinus) and/or Aphis medicaginis (Aphis craccivora Koch), etc.

### Compositions and Formulations of the Compounds of the Invention

The compound of the present invention usually can be used as an active ingredient of pesticides in a composition, i.e. in a formulation typically also comprising an agriculturally acceptable surfactant and/or carrier.

The above surfactant may be various surfactants as known in the field of pesticide formulations, such as anionic surfactants, cationic surfactants, nonionic surfactants and amphoteric surfactants, block polymers, polyelectrolytes and their mixtures. These surfactants may be used as emulsifiers, dispersants, wetting agents, penetration enhancers or adjuvants.

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are homo- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

The above carriers may be various carriers known in the field of pesticide formulations and comprise silicates, carbonates, sulfates, oxides, phosphates, plant carriers and synthetic carriers. Specifically, such as one or more of white carbon, kaolin, diatomite, clay, talc, organic bentonite, pumice stone, titanium dioxide, dextrin, cellulose powder, light calcium carbonate, soluble starch, corn starch, sawdust powder, urea, amine fertilizer, a mixture of urea and amine fertilizer, glucose, maltose, sucrose, anhydrous potassium carbonate, anhydrous sodium carbonate, anhydrous potassium bicarbonate, anhydrous sodium bicarbonate, attapulgite, a mixture of anhydrous potassium carbonate and anhydrous potassium bicarbonate, and a mixture of anhydrous sodium carbonate and anhydrous sodium bicarbonate.

According to the present invention, the pesticide composition may also contain various formulation auxiliaries commonly used in the field of pesticide formulations. Specifically, the formulation auxiliaries may be one or more of solvents, additives, thickeners, antifreezes, capsule materials, protective agents, defoamers, disintegrants, stabilizers, preservatives, adhesives and chelating agents.

Suitable solvents are water and organic solvents such as mineral oil fractions with medium to high boiling point, for example kerosene, diesel oil; oils of vegetable or animal origin; aliphatic, cyclic and aromatic hydrocarbons, for example toluene, paraffin, tetrahydro-naphthalene, alkylated naphthalenes; alcohols, for example ethanol, propanol, butanol, benzyl alcohol, cyclohexanol; glycols; DMSO; ketones, for example cyclohexanone; esters, for example lactate ester, carbonates, fatty acid esters, γ-butyrolactone; fatty acids; phosphonates; amines; amides, for example N-methylpyrrolidone, fatty acid dimethylamide; and mixtures thereof.

The above solvents may also be used as cosolvents.

Suitable thickeners are selected from polysaccharides (such as xanthan gum, carboxymethyl cellulose), inorganic clays (organic modified or unmodified), polycarboxylates and silicates.

Suitable antifreezes are selected from ethylene glycol, propylene glycol, glycerol, urea, glycerol and mixtures thereof.

Suitable capsule materials are selected from polyurethane, polyurea, urea-formaldehyde resin and mixtures thereof.

Suitable protective agents are selected from polyvinyl alcohol and/or polyethylene glycol.

Suitable defoamers are selected from polysiloxane, silicone emulsion, long chain alcohols, fatty acids and their salts, and fluorinated organic compounds and mixtures thereof.

Suitable disintegrating agents are selected from bentonite, urea, ammonium sulfate, aluminium chloride, citric acid, succinic acid, sodium bicarbonate and mixtures thereof.

Suitable stabilizers are selected from triphenyl phosphite, epichlorohydrin, acetic anhydride and mixtures thereof.

Suitable preservatives are selected from benzoic acid, sodium benzoate, 1,2-benzoisothiazoline-3-one (BITf for short), casson, potassium sorbate and mixtures thereof.

Suitable adhesive are selected from polyvinylpyrrolidone, polyvinyl acetate, polyvinyl alcohol, polyacrylate, biowax or synthetic wax and cellulose ether.

The pesticides of the invention may be applied in the form of their formulations or the use forms prepared therefrom; the use forms are such as aerosol, capsule suspension, cold-fogging concentrate, hot fogging concentrate, encapsulating granules, fine granules, flowable concentrate for seed treatment, ready-to-use solution, dustable powder, emulsifiable concentrates, oil in water emulsions, water in oil emulsions, large granules, micro granules, oil dispersible powder, oil miscible flowable concentrates, oil miscible liquids, gas agents (under pressure), gas generant products, foaming agents, paste, suspension concentrate, suspoemulsion concentrate, soluble concentrates, suspensions, wettable powder, soluble powder, powder and granules, water-soluble and water-dispersible granules or tablets, water-soluble or water-dispersible powders for seed treatment, wettable powders, natural products impregnated with active substances and synthetic substances, microcapsules in polymers and seed coating materials, and ULV (ultra low volume) cold and hot fogging formulations.

The formulation may optionally include compositions containing other pesticide compounds. The compounds of the invention can be combined with other pesticides, including insecticides, nematocides, acaricides, arthropodicides or combinations thereof. The other pesticides are compatible with the compounds of the invention in the medium selected for application and do not antagonize the activities of the compounds of the invention to form pesticide mixtures and their synergistic mixtures.

In addition, the agricultural chemical compounds may also be fungicide, herbicide, bactericide, attractant, growth regulator, fertilizer, safety agent, sterilant or combinations thereof, in which the agricultural chemical compound is compatible with the compound of the invention in the medium selected for application and does not antagonize the activity of the compound of the invention. Therefore, in the embodiment, the other agricultural chemical compounds are used as additive toxicants for the same or different purposes.

The composition of the compound of the invention can be used to treat the plant and plant parts directly, or be used by the conventional treatment method acting on the surrounding environment, habitat or storage space around the plant and plant parts, and the conventional treatment method is, for example, coating one or more layers on the plant reproductive materials, especially for seeds through dipping, spraying, misting, irrigation, evaporation, powder spreading, atomizing, sowing, foaming, spreading, coating, watering, dripping, also through a dry seed treatment, wet seed treatment, slurry treatment, crusting, etc. The active substance can also be effectively utilized by an ultra-low volume method or by injecting the compound formulation of the invention or the compound into the soil.

### Use of the Compounds and Pharmaceutical Compositions

The present invention relates to non-restrictive examples of pests that are controlled or prevented to attack useful plants, including:

Hemiptera: Delphacidae such as Nilaparvata lugens, Laodelphax striatellus; Deltocephalidae such as Nephotettix cincticeps; Aphididae such as Aphis gossypii, Aphis craccivora Koch; Pentatomidae such as Nezara antennata; Aleyrodidae such as Trialeurodes vaporariorum; Coccidae such as Calformia red scale (Aonidiella aurantii); Tingidae; Homoptera (Psyllidea);

Lepidoptera: Pyralidae such as Chilo suppressalis; Noctuidae such as Spodoptera litura, Pseudaletia separata, Heliothis spp. and Helicoverpa spp.; Pieridae such as Pieris rapae; Tortricidae such as Adoxophyes; Gracillariidae such as Caloptilia theivora and Phyllonorycter ringoneella; Carposinidae such as Carposina niponensis; Lyonetiidae such as Lyonetia spp.; Lymantriidae such as Lymantria spp. and Euproctis spp.; Yponomeutidae such as Plutella xylostella; Gelechiidae such as Pectinophora gossypiella and Phthorimaea operculella; Arctiidae such as Hyphantria cunea; and Tineidae such as Tineatranslucens and Tineola bisselliella;

Thysanoptera: Frankliniella occidentalis, Thrips palmi, Scirtothrips dorsalis, Thrips tabaci, Frankliniella intonsa and Frankliniella fusca;

Diptera: Musca domestica, Culex popiens pallens, Tabanus trigonus, Hylemya anitqua, Hylemya platura, Anopheles sinensis, Agromyza oryzae, Hydrellia griseola, Chlorops oryzae, Dacus cucurbitae, Ceratitis capitata and Liriomyza trifolii;

Coleoptera: Epilachna vigintioctopunctata, Phyllotreta striolata, Oulema oryzae, Echinocnemus squameus, Lissorhoptrus oryzophilus, Anthonomus grandis, Callosobruchus chinensis, Sphenophorus venatus, Popillia japonica, Anomala cuprea, Diabrotica spp., Leptinotarsa decemlineata, Agriotes spp., Lasioderma serricorne, Anthrenus verbasci, Tribolium castaneum, Lyctus brunneus, Anoplophora malasiaca and Tomicus piniperda;

Orthoptera: Locusta migratoria, Gryllotalpa afficana, Oxya yezoensis and Oxya japanica;

Hymenoptera: Athalia rosae, Acromyrmex spp. and Solenopsis spp.;

Nematodes: Aphelenchoides besseyi, Nothotylenchus acris, Heterodera glycines, Meloidogyne incognita, Pratylenchus penetrans and Nacobbus aberrans;

Blattariae: Blattella germanica, Periplaneta fuliginosa, Periplaneta Americana, Periplaneta brunnea and Blatta orientalis;

Acarina: Tetranychidae (such as Tetranychus cinnabarinus, Tetranychusurticae, Panonychus citri and Oligonychus spp.); Eriophyidae (such as Aculops pelekassi); Tarsonemidae; Tenuipalpidae; Tuckerellidae; Tuckerellidae Acaridae; Pyroglyphidae (such as Dermatophagoides farinae and Dermatophagoides ptrenyssnus); Cheyletidae, Cheyletus malaccensis and Cheyletus moorei; and Dermanyssidae.

Within the scope of the invention, useful plants include the following plant species: grain (wheat, barley, rye, oat, rice, corn, sorghum and related species); beet (Beta vulgaris and mangelwurzel); pome, drupe and soft fruit (apple, pear, plum, peach, almond, cherry, strawberry, raspberry and blackberry); leguminous plants (lentils, beans, peas, peas, soybeans); oil crops (rape, mustard, olive, sunflower, coconut, castor oil plant, cocoa bean, peanut or soybean); melon plants (pumpkin, cucumber, melon); fiber plants (cotton, flax, hemp, jute); citrus fruits (orange, lemon, grapefruit, citrus); vegetables (spinach, lettuce, asparagus, broccoli, carrot, onion, tomato, potato, cayenne pepper); laurel plants (avocado, cinnamomum, camphor) or plants, such as tobacco, nuts, coffee, eggplant, sugar cane, tea, pepper, grapevine, hops, bananas and natural rubber plants, and turf, ornamental and forest plants, such as flowers, shrubs, broad-leaved or evergreen trees, such as conifers, and plant reproductive materials.

The term “plant reproductive material” should be understood to denote the reproductive parts of the plant, such as seeds, which can be used for the reproduction of the plant, as well as nutritive reproductive materials such as cuttings or tubers (e.g. potatoes).

The compound or composition of the present invention can kill pests through an active substance at an effective amount. Therefore, the invention also relates to a method of controlling pests, the method comprises applying the active ingredients or compositions of the invention to seeds, plants or plant parts, fruits, or soil where plants grow. The application can be carried out before and/or after the infestation of pests on seeds, plant or plant parts, fruits or soil where plants grow.

The term “effective amount” refers to an amount of the compound or composition of the invention that is sufficient to control pests on cultivated plants or in material protection without causing significant damage to treated plants. The amount can vary over a wide range depending on various factors such as pest species, treated cultivated plants or materials, climatic conditions and the specific used compounds.

The compound or composition of the invention has a simple use method, and the compound or composition of the invention is applied to the pest or its growth medium. The application dose of the compound or composition of the invention varies according to weather conditions, dosage forms, application timing, application method, application area, target diseases, target crops, etc.

### General Synthetic Procedures

In the present invention, if the chemical name of the compound doesn't match the corresponding structure, the compound is characterized by the corresponding structure. Generally, the compounds disclosed herein may be prepared by methods described herein, wherein the substituents are as defined for Formula (I) above, except where further noted.

Persons skilled in the art will recognize that the chemical reactions described may be readily adapted to prepare a number of other compounds disclosed herein, and alternative methods for preparing the compounds disclosed herein are deemed to be within the scope disclosed herein. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds disclosed herein.

The following schemes describe the preparation procedure of the compound disclosed herein.

### Schemes

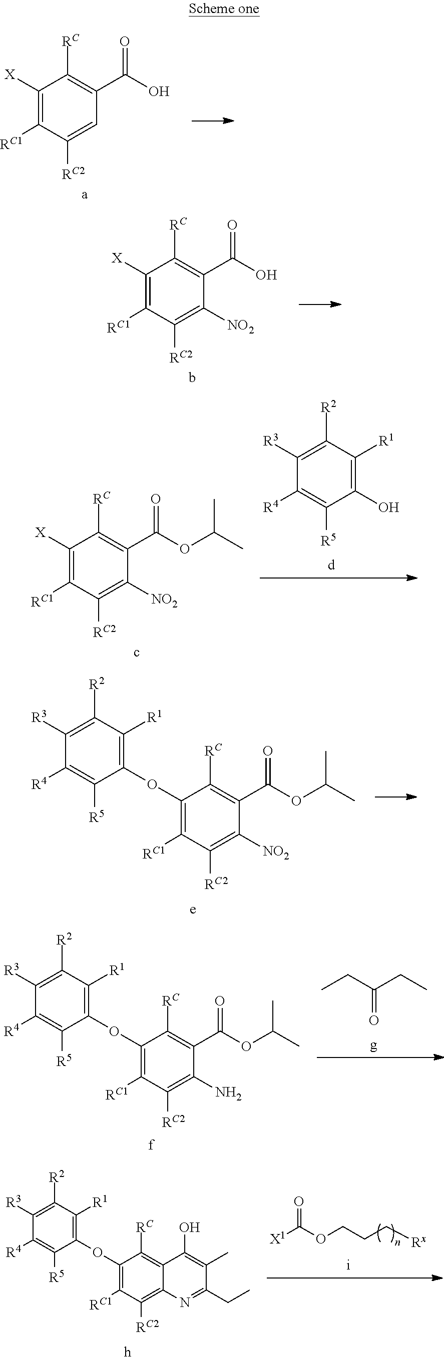

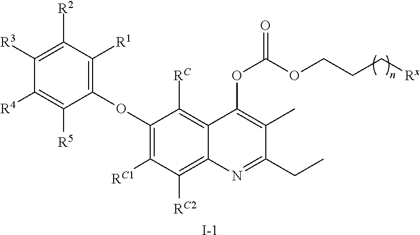

Compound I-1 disclosed herein can be prepared by the process illustrated in scheme one. Compound a can be converted to compound b through a nitrification reaction; compound b can be reacted with isopropanol by an esterification reaction to form compound c; compound c can be reacted with compound d under a base (such as sodium hydroxide, sodium hydride, potassium carbonate, etc.) by a substitution reaction to form compound e; compound e can be reduced under a reducer (such as iron powder, hydrogen, etc.) by a reduction reaction to form compound f; compound f can be reacted with compound g in the present of a Lewis acid (such as Zinc chloride, aluminium chloride, etc.) by a quinoline cyclization reaction to form compound h; compound h can be reacted with compound i under a base (such as pyridine, sodium hydride, triethylamine, etc.) by an esterification reaction to form target compound I-1;

Wherein R¹, R², R³, R⁴, R⁵, R^{C}, R^{C1}, R^{C2}, R^{x} and n have the definitions as described herein; each X and X¹ is independently halo.

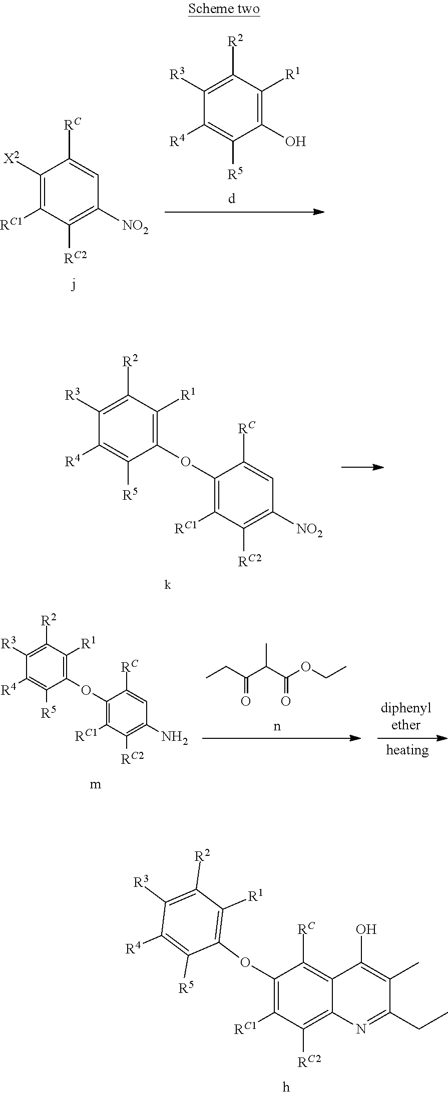

Compound h can be prepared by the process illustrated in scheme two. Compound j can be reacted with compound d under a base (such as sodium hydroxide, sodium hydride, potassium carbonate, etc.) by a substitution reaction to form compound k; compound k can be reduced under a reducer (such as iron powder, hydrogen, etc.) by a reduction reaction to form compound m; compound m can be reacted with compound n to form a compound, the compound can be converted to compound h under heating (temperature 200-280° C.) in diphenyl ether system;

Wherein R¹, R², R³, R⁴, R⁵, R^{C}, R^{C1} and R^{C2} have the definitions as described herein; X² is halo.

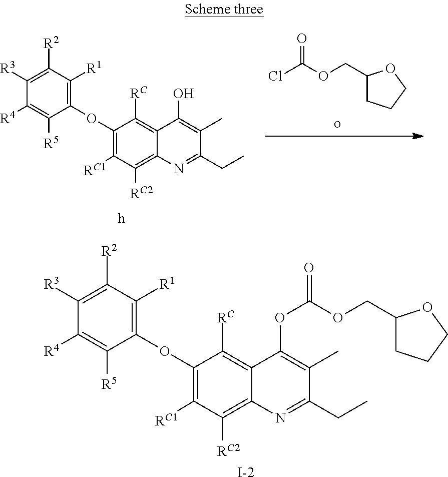

Target compound I-2 can be prepared by the process illustrated in scheme three. Compound h can be reacted with compound o under a base (such as pyridine, sodium hydride, triethylamine, etc.) to form target compound I-2;

Wherein R¹, R², R³, R⁴, R⁵, R^{C}, R^{C1} and R^{C2} have the definitions as described herein.

### SPECIFIC EMBODIMENTS

The following examples are used for illustrating the invention, but cannot be construed to limit the scope of the invention.

### EXAMPLES

Using parts of the compounds of the invention as examples, the preparations of the compounds of the present invention have been described in detail in the following examples.

### Intermediate O: synthesis of isopropyl 5-fluoro-4-methyl-2-nitrobenzoate

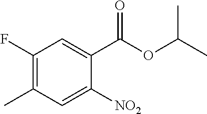 [SMILES: Cc1cc([N+](=O)[O-])c(C(=O)OC(C)C)cc1F]

### Step 1: synthesis of 5-fluoro-4-methyl-2-nitrobenzoic acid

Nitric acid (20 mL, 65%) and sulfuric acid (50 mL, 98%) were added into a 250 mL round-bottom flask, the flask was cooled to −10° C. in an ice-salt bath, a solution of 3-fluoro-4-methylbenzoic acid (15.00 g, 97.4 mmol) in concentrated sulfuric acid (50 mL) was added dropwise slowly to the above mixed acid, the reaction system temperature was controlled between −10 and 0° C. After the addition, the ice-salt bath was removed, the mixture was stirred at 25° C. for 2.5 hours. The crude product was poured into ice water, and the precipitate was filtered off and dried, and then recrystallized from toluene to give a white solid (15.91 g, 82.1%).

LC-MS: (M+1) m/z=200.0.

### Step 2: synthesis of isopropyl 5-fluoro-4-methyl-2-nitrobenzoate

5-Fluoro-4-methyl-2-nitrobenzoic acid (10.0 g, 50.2 mmol) was dissolved in isopropanol (60 mL), and then concentrated sulfuric acid (10 mL, 98%) was added at 0° C. slowly. After the addition, the mixture was heated to 105° C. and refluxed for 20 hours. The excess isopropanol was removed under vacuum using a rotary evaporator, the resulting mixture was extracted with ethyl acetate (60 mL×3), the organic layers were combined and washed with water (30 mL) and saturated aqueous NaCl (30 mL), and then dried over anhydrous magnesium sulfate and concentrated using a rotary evaporator to give a red liquid (9.63 g, 79.6%).

¹H NMR (400 MHz, CDCl₃) δ (ppm): 7.81 (d, 1H), 7.33 (d, 1H), 5.26 (m, 1H), 2.39 (s, 3H), 1.35 (d, 6H);

LC-MS: (M+1) m/z=242.2.

### Example 1: synthesis of 2-ethyl-3,7-dimethyl-6-(4-(trifluoromethoxy)phenoxy)quinolin-4-yl (2-methoxyethyl)carbonate

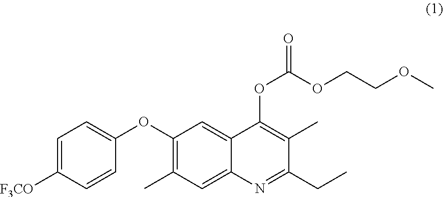 [SMILES: CCc1nc2cc(C)c(Oc3ccc(OC(F)(F)F)cc3)cc2c(OC(=O)OCCOC)c1C]

### Step 1: Synthesis of isopropyl 4-methyl-2-nitro-5-(4-(trifluoromethoxy)phenoxy)benzoate

Isopropyl 5-fluoro-4-methyl-2-nitrobenzoate (9.60 g, 39.8 mmol), p-trifluoromethoxy phenol (7.12 g, 40.0 mmol) and potassium carbonate (5.52 g, 40.0 mmol) were added into a 250 mL round-bottomed flask, and then N,N-dimethylformamide (100 mL) was added, the mixture was heated to 80° C. quickly and stirred for 18 hours under N₂. To the mixture was added ice-water (100 mL), the resulting mixture was extracted with ethyl acetate (60 mL×3), the organic layers were combined and washed with saturated aqueous sodium bicarbonate solution (50 mL×3), dried over anhydrous magnesium sulfate, and then filtered, the filtrate was concentrated using a rotary evaporator to give a yellow oil (12.0 g, 75.6%).

LC-MS: (M+1) m/z=400.3.

### Step 2: synthesis of isopropyl 2-amino-4-methyl-5-(4-(trifluoromethoxy)phenoxy)benzoate

Under N₂, iron powder (5.60 g, 100.0 mmol), ethanol (100 mL), water (20 mL) and hydrochloric acid (1.0 mL, 35%) were added into a 500 mL round-bottomed flask, the mixture was heated to 90° C. and stirred, a solution of isopropyl 4-methyl-2-nitro-5-(4-(trifluoromethoxy)phenoxy)benzoate (12.0 g, 30.0 mmol) in ethanol (100 mL) was added dropwise slowly. After the addition, the mixture was stirred at 90° C. for 12 hours. The solid impurities were removed by filtration and the filtrate was concentrated using a rotary evaporator, the residue was diluted with ethyl acetate (150 mL), and the resulting solution was washed with brine (30 mL×3), dried over anhydrous magnesium sulfate, and then filtered, the filtrate was concentrated using a rotary evaporator, the residue was purified on a silica gel column [PE/EtOAc (v/v)=10/1] to give a yellow oil (10.5 g, 94.6%).

¹H NMR (400 MHz, CDCl₃) δ (ppm): 7.60-7.51 (m, 1H), 7.25-7.15 (m, 2H), 6.91-6.80 (m, 2H), 6.20 (s, 1H), 5.69 (s, 2H), 5.21 (m, 1H), 2.11 (s, 3H), 1.35 (d, 6H);

¹⁹F NMR (100 MHz, CDCl₃) δ (ppm): −58.4;

LC-MS: (M+1) m/z=370.3.

### Step 3: synthesis of 2-ethyl-3,7-dimethyl-6-(4-(trifluoromethoxy)phenoxy)quinolin-4-ol

Under N₂, isopropyl 2-amino-4-methyl-5-(4-(trifluoromethoxy)phenoxy)benzoate (10.0 g, 27.1 mmol), 3-pentone (4.3 g, 50.0 mmol), zinc chloride (6.82 g, 50.0 mmol) and xylene (100 mL) were added into a 500 mL round-bottomed flask, the mixture was refluxed at 160° C. for 18 hours. After the reaction was completed, the xylene was removed using a rotary evaporator to obtain a yellowish-brown viscous substance, the viscous substance was cooled to 25° C. and diluted with water (50 mL) and ethyl acetate (50 mL), the mixture was stirred to form suspension and filtered to give a gray white solid (6.76 g, 66.2%).

¹H NMR (400 MHz, CDCl₃) δ (ppm): 11.4 (s, 1H), 7.51 (s, 1H), 7.28 (s, 1H), 7.22 (m, 2H), 7.01 (m, 2H), 2.72 (m, 2H), 2.31 (s, 3H), 2.03 (s, 3H), 1.25 (m, 3H);

LC-MS: (M+1) m/z=378.1.

### Step 4: synthesis of 2-ethyl-3,7-dimethyl-6-(4-(trifluoromethoxy)phenoxy)quinolin-4-yl (2-methoxyethyl)carbonate

Under N₂, 2-ethyl-3,7-dimethyl-6-(4-(trifluoromethoxy)phenoxy)quinolin-4-ol (0.600 g, 1.6 mmol), triethylamine (1.0 mL, 7.2 mmol) and dichloromethane (10 mL) were added into a 50 mL round-bottomed flask, the mixture was cooled to 0° C. in an ice-salt bath, and then ethyl 2-methoxychloroformate (0.28 g, 2.0 mmol) was added, the resulting mixture was stirred for 12 hours. The solvent was removed using a rotary evaporator, the residue was diluted with water (30 mL) and the resulting mixture was extracted with ethyl acetate (30 mL), the organic layer was dried over magnesium sulfate and concentrated using a rotary evaporator to give a yellow liquid (0.633 g, 82.7%).

¹H NMR (400 MHz, CDCl₃) δ (ppm): 7.96 (s, 1H), 7.22 (s, 1H), 7.19 (d, 2H), 6.99 (m, 2H), 4.40 (m, 2H), 3.65 (m, 2H), 3.39 (s, 3H), 3.02 (q, 2H), 2.42 (s, 3H), 2.33 (s, 3H), 1.39 (t, 3H);

LC-MS: (M+1) m/z=480.1.

### Example 2: Synthesis of 2-ethyl-3,7-dimethyl-6-(p-tolyloxy)quinolin-4-yl (2-methoxyethyl) carbonate

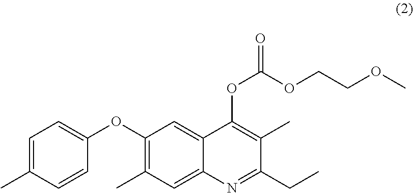

### Step 1: synthesis of isopropyl 4-methyl-2-nitro-5-(p-tolyloxy)benzoate

Isopropyl 5-fluoro-4-methyl-2-nitrobenzoate (9.60 g, 39.8 mmol), p-methylphenol (7.12 g, 66.0 mmol) and potassium carbonate (5.52 g, 40.0 mmol) were added into a 250 mL round-bottomed flask, and then N,N-dimethylformamide (100 mL) was added, the mixture was heated to 80° C. quickly and stirred for 18 hours under N₂. To the mixture was added ice-water (100 mL), the resulting mixture was extracted with ethyl acetate (60 mL×3), the organic layers were combined and washed with saturated aqueous sodium bicarbonate solution (50 mL×3), dried over anhydrous magnesium sulfate, and then filtered, the filtrate was concentrated using a rotary evaporator to give a yellow oil (11.2 g, 86.1%).

LC-MS: (M+1) m/z=330.1.

### Step 2: Synthesis of isopropyl 2-amino-4-methyl-5-(p-tolyloxy)benzoate

Under N₂, iron powder (5.60 g, 100.0 mmol), ethanol (100 mL), water (20 mL) and hydrochloric acid (1.0 mL, 35%) were added into a 500 mL round-bottomed flask, the mixture was heated to 90° C. and stirred, a solution of isopropyl 4-methyl-2-nitro-5-(p-methylphenoxy)benzoate (10.5 g, 36.4 mmol) in ethanol (100 mL) was added dropwise slowly. After the addition, the mixture was stirred at 90° C. for 12 hours. The solid impurities were removed by filtration and the filtrate was concentrated using a rotary evaporator, the residue was diluted with ethyl acetate (150 mL), and the resulting solution was washed with brine (30 mL×3), the organic layers were combined and dried over anhydrous magnesium sulfate, and then filtered, the filtrate was concentrated using a rotary evaporator, the residue was purified on a silica gel column [PE/EtOAc (v/v)=10/1] to give a yellow oil (9.3 g, 96.8%).

LC-MS: (M+1) m/z=300.1.

### Step 3: synthesis of 2-ethyl-3,7-dimethyl-6-(p-tolyloxy)quinolin-4-ol

Under N₂, isopropyl 2-amino-4-methyl-5-(p-methylphenoxy)benzoate (9.0 g, 33.4 mmol), 3-pentone (4.3 g, 50.0 mmol), zinc chloride (6.82 g, 50.0 mmol) and xylene (100 mL) were added into a 500 mL round-bottomed flask, the mixture was refluxed at 160° C. for 18 hours. After the reaction was completed, the xylene was removed using a rotary evaporator to obtain a yellowish-brown viscous substance, the viscous substance was cooled to 25° C. and diluted with water (50 mL) and ethyl acetate (50 mL), the mixture was stirred to form suspension and filtered to give a gray white solid (5.46 g, 59.1%).

LC-MS: (M+1) m/z=308.1.

### Step 4: synthesis of 2-ethyl-3,7-dimethyl-6-(p-tolyloxy)quinolin-4-yl (2-methoxyethyl) carbonate

Under N₂, 2-ethyl-3,7-dimethyl-6-(p-methylphenoxy)quinolin-4-ol (0.95 g, 1.9 mmol), triethylamine (1.0 mL, 7.2 mmol) and dichloromethane (10 mL) were added into a 50 mL round-bottomed flask, the mixture was cooled to 0° C. in an ice-salt bath, and then ethyl 2-methoxychloroformate (0.28 g, 2.0 mmol) was added, the resulting mixture was stirred for 12 hours. The solvent was removed using a rotary evaporator, the residue was diluted with water (30 mL) and the resulting mixture was extracted with ethyl acetate (30 mL), the organic layer was dried over magnesium sulfate and concentrated using a rotary evaporator to give a yellow liquid (0.843 g, 66.9%).

¹H NMR (400 MHz, CDCl₃) δ (ppm): 7.93 (s, 1H), 7.28 (s, 1H), 7.14 (d, 2H), 6.88 (m, 2H), 4.38 (t, 2H), 3.63 (t, 2H), 3.39 (s, 3H), 3.02 (q, 2H), 2.44 (s, 3H), 2.35 (s, 3H), 2.32 (s, 3H), 1.36 (t, 3H);

LC-MS: (M+1) m/z=410.1.

### Example 3: synthesis of 2-ethyl-6-(4-methoxyphenoxy)-3,7-dimethylquinolin-4-yl (2-methoxyethyl)carbonate

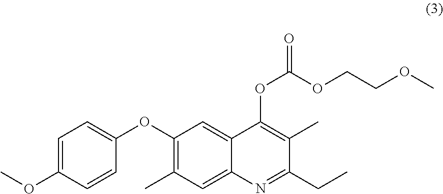

### Step 1: synthesis of isopropyl 5-(4-methoxyphenoxy)-4-methyl-2-nitrobenzoate

Isopropyl 5-fluoro-4-methyl-2-nitrobenzoate (9.60 g, 39.8 mmol), p-methylphenol (7.12 g, 57.4 mmol) and potassium carbonate (5.52 g, 40.0 mmol) were added into a 250 mL round-bottomed flask, and then N,N-dimethylformamide (100 mL) was added, the mixture was heated to 80° C. quickly and stirred for 18 hours under N₂. To the mixture was added ice-water (100 mL), the resulting mixture was extracted with ethyl acetate (60 mL×3), the organic layers were washed with saturated aqueous sodium bicarbonate solution (50 mL×3), and then the organic layers were combined and dried over anhydrous magnesium sulfate, and then filtered, the filtrate was concentrated using a rotary evaporator to give a yellow oil (12.8 g, 92.7%).

LC-MS: (M+1) m/z=346.1.

### Step 2: Synthesis of isopropyl 2-amino-5-(4-methoxyphenoxy)-4-methylbenzoate

Under N₂, iron powder (5.60 g, 100.0 mmol), ethanol (100 mL), water (20 mL) and hydrochloric acid (1.0 mL, 35%) were added into a 500 mL round-bottomed flask, the mixture was heated to 90° C. and stirred, a solution of isopropyl 5-(4-methoxyphenoxy)-4-methyl-2-nitrobenzoate (12.5 g, 30.0 mmol) in ethanol (100 mL) was added dropwise slowly. After the addition, the mixture was stirred at 90° C. for 12 hours. The solid impurities were removed by filtration and the filtrate was concentrated using a rotary evaporator, the residue was diluted with ethyl acetate (150 mL), and the resulting solution was washed with brine (30 mL×3), the organic layers were combined and dried over anhydrous magnesium sulfate, and then filtered, the filtrate was concentrated using a rotary evaporator, the residue was purified on a silica gel column [PE/EtOAc (v/v)=10/1] to give a yellow oil (9.3 g, 81.6%).

LC-MS: (M+1) m/z=316.1.

### Step 3: synthesis of 2-ethyl-6-(4-methoxyphenoxy)-3,7-dimethylquinolin-4-ol

Under N₂, isopropyl 2-amino-5-(4-methoxyphenoxy)-4-methylbenzoate (8.0 g, 27.1 mmol), 3-pentone (4.3 g, 50.0 mmol), zinc chloride (6.82 g, 50.0 mmol) and xylene (100 mL) were added into a 500 mL round-bottomed flask, the mixture was refluxed at 160° C. for 18 hours. After the reaction was completed, the xylene was removed using a rotary evaporator to obtain a yellowish-brown viscous substance, the viscous substance was cooled to 25° C. and diluted with water (50 mL) and ethyl acetate (50 mL), the mixture was stirred to form suspension and filtered to give a gray white solid (5.96 g, 72.7%).

LC-MS: (M+1) m/z=324.2.

### Step 4: synthesis of 2-ethyl-6-(4-methoxyphenoxy)-3,7-dimethylquinolin-4-yl (2-methoxyethyl)carbonate

Under N₂, 2-ethyl-6-(4-methoxyphenoxy)-3,7-dimethylquinolin-4-ol (0.850 g, 1.6 mmol), triethylamine (1.0 mL, 7.2 mmol) and dichloromethane (10 mL) were added into a 50 mL round-bottomed flask, the mixture was cooled to 0° C. in an ice-salt bath, and then ethyl 2-methoxychloroformate (0.28 g, 2.0 mmol) was added, the resulting mixture was stirred for 12 hours. The solvent was removed using a rotary evaporator, the residue was diluted with water (30 mL) and the resulting mixture was extracted with ethyl acetate (30 mL), the organic layer was dried over magnesium sulfate and concentrated using a rotary evaporator to give a yellow liquid (0.753 g, 67.2%).

¹H NMR (400 MHz, CDCl₃) δ (ppm): 7.95 (s, 1H), 7.22 (s, 1H), 7.13 (d, 2H), 6.89 (m, 2H), 4.41 (t, 2H), 3.73 (s, 3H), 3.64 (t, 2H), 3.40 (s, 3H), 3.01 (q, 2H), 2.43 (s, 3H), 2.33 (s, 3H), 1.37 (t, 3H);

LC-MS: (M+1) m/z=426.1.

### Example 4: synthesis of 6-(4-(tert-butyl)phenoxy)-2-ethyl-3,7-dimethylquinolin-4-yl (2-methoxyethyl)carbonate

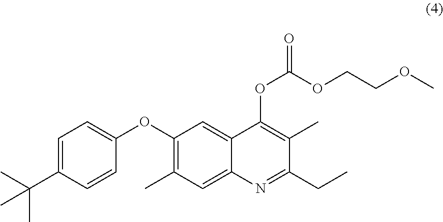 [SMILES: CCc1nc2cc(C)c(Oc3ccc(C(C)(C)C)cc3)cc2c(OC(=O)OCCOC)c1C]

### Step 1: synthesis of isopropyl 5-(4-(tert-butyl)phenoxy)-4-methyl-2-nitrobenzoate

Isopropyl 5-fluoro-4-methyl-2-nitrobenzoate (9.60 g, 39.8 mmol), p-(tert-butyl)phenol (7.12 g, 47.4 mmol) and potassium carbonate (5.52 g, 40.0 mmol) were added into a 250 mL round-bottomed flask, and then N,N-dimethylformamide (100 mL) was added, the mixture was heated to 80° C. quickly and stirred for 18 hours under N₂. To the mixture was added ice-water (100 mL), the resulting mixture was extracted with ethyl acetate (60 mL×3), the organic layers were washed with saturated aqueous sodium bicarbonate solution (50 mL×3), and then the organic layers were combined and dried over anhydrous magnesium sulfate, and then filtered, the filtrate was concentrated using a rotary evaporator to give a yellow oil (10.8 g, 72.9%).

LC-MS: (M+1) m/z=372.4.

### Step 2: Synthesis of isopropyl 2-amino-5-(4-(tert-butyl)phenoxy)-4-methylbenzoate

Under N₂, iron powder (5.60 g, 100.0 mmol), ethanol (100 mL), water (20 mL) and hydrochloric acid (1.0 mL, 35%) were added into a 500 mL round-bottomed flask, the mixture was heated to 90° C. and stirred, a solution of isopropyl 5-(4-(tert-butyl)phenoxy)-4-methyl-2-nitrobenzoate (10.0 g, 30.0 mmol) in ethanol (100 mL) was added dropwise slowly. After the addition, the mixture was stirred at 90° C. for 12 hours. The solid impurities were removed by filtration and the filtrate was concentrated using a rotary evaporator, the residue was diluted with ethyl acetate (150 mL), and the resulting solution was washed with brine (30 mL×3), the organic layers were combined and dried over anhydrous magnesium sulfate, and then filtered, the filtrate was concentrated using a rotary evaporator, the residue was purified on a silica gel column [PE/EtOAc (v/v)=10/1] to give a yellow oil (7.5 g, 81.5%).

LC-MS: (M+1) m/z=342.4.

### Step 3: synthesis of 6-(4-(tert-butyl)phenoxy)-2-ethyl-3,7-dimethylquinolin-4-ol

Under N₂, isopropyl 2-amino-5-(4-(tert-butyl)phenoxy)-4-methylbenzoate (9.0 g, 27.1 mmol), 3-pentone (4.3 g, 50.0 mmol), zinc chloride (6.82 g, 50.0 mmol) and xylene (100 mL) were added into a 500 mL round-bottomed flask, the mixture was refluxed at 160° C. for 18 hours. After the reaction was completed, the xylene was removed using a rotary evaporator to obtain a yellowish-brown viscous substance, the viscous substance was cooled to 25° C. and diluted with water (50 mL) and ethyl acetate (50 mL), the mixture was stirred to form suspension and filtered to give a gray white solid (6.77 g, 73.5%).

LC-MS: (M+1) m/z=350.2.

### Step 4: synthesis of 6-(4-(tert-butyl)phenoxy)-2-ethyl-3,7-dimethylquinolin-4-yl (2-methoxyethyl)carbonate

Under N₂, 6-(4-(tert-butyl)phenoxy)-2-ethyl-3,7-dimethylquinolin-4-ol (0.900 g, 1.6 mmol), triethylamine (1.0 mL, 7.2 mmol) and dichloromethane (10 mL) were added into a 50 mL round-bottomed flask, the mixture was cooled to 0° C. in an ice-salt bath, and then ethyl 2-methoxychloroformate (0.28 g, 2.0 mmol) was added, the resulting mixture was stirred for 12 hours. The solvent was removed using a rotary evaporator, the residue was diluted with water (30 mL) and the resulting mixture was extracted with ethyl acetate (30 mL), the organic layer was dried over magnesium sulfate and concentrated using a rotary evaporator to give a yellow liquid (0.831 g, 71.6%).

¹H NMR (400 MHz, CDCl₃) δ (ppm): 7.96 (s, 1H), 7.21 (s, 1H), 7.14 (d, 2H), 6.90 (m, 2H), 4.42 (t, 2H), 3.66 (t, 2H), 3.38 (s, 3H), 3.03 (q, 2H), 2.42 (s, 3H), 2.32 (s, 3H), 1.40 (t, 3H), 1.34 (s, 9H);

LC-MS: (M+1) m/z=452.2.

### Example 5: synthesis of 6-(3,5-difluorophenoxy)-2-ethyl-3,7-dimethylquinolin-4-yl (2-methoxyethyl)carbonate

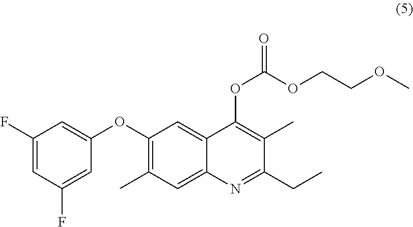 [SMILES: CCc1nc2cc(C)c(Oc3cc(F)cc(F)c3)cc2c(OC(=O)OCCOC)c1C]

### Step 1: Synthesis of isopropyl 5-(3,5-difluorophenoxy)-4-methyl-2-nitrobenzoate

Isopropyl 5-fluoro-4-methyl-2-nitrobenzoate (9.60 g, 39.8 mmol), 3,5-difluorophenol (7.12 g, 54.8 mmol) and potassium carbonate (5.52 g, 40.0 mmol) were added into a 250 mL round-bottomed flask, and then N,N-dimethylformamide (100 mL) was added, the mixture was heated to 80° C. quickly and stirred for 18 hours under N₂. To the mixture was added ice-water (100 mL), the resulting mixture was extracted with ethyl acetate (60 mL×3), the organic layers were washed with saturated aqueous sodium bicarbonate solution (50 mL×3), and then the organic layers were combined and dried over anhydrous magnesium sulfate, and then filtered, the filtrate was concentrated using a rotary evaporator to give a yellow oil (10.2 g, 72.8%).

LC-MS: (M+1) m/z=352.1.

### Step 2: synthesis of isopropyl 2-amino-5-(3,5-difluorophenoxy)-4-methylbenzoate

Under N₂, iron powder (5.60 g, 100.0 mmol), ethanol (100 mL), water (20 mL) and hydrochloric acid (1.0 mL, 35%) were added into a 500 mL round-bottomed flask, the mixture was heated to 90° C. and stirred, a solution of isopropyl 5-(3,5-difluorophenoxy)-4-methyl-2-nitrobenzoate (10.0 g, 30.0 mmol) in ethanol (100 mL) was added dropwise slowly. After the addition, the mixture was stirred at 90° C. for 12 hours. The solid impurities were removed by filtration and the filtrate was concentrated using a rotary evaporator, the residue was diluted with ethyl acetate (150 mL), and the resulting solution was washed with brine (30 mL×3), the organic layers were combined and dried over anhydrous magnesium sulfate, and then filtered, the filtrate was concentrated using a rotary evaporator, the residue was purified on a silica gel column [PE/EtOAc (v/v)=10/1] to give a yellow oil (7.5 g, 82.4%).

LC-MS: (M+1) m/z=322.1.

### Step 3: synthesis of 6-(3,5-difluorophenoxy)-2-ethyl-3,7-dimethylquinolin-4-ol

Under N₂, isopropyl 2-amino-5-(3,5-difluorophenoxy)-4-methylbenzoate (8.0 g, 27.1 mmol), 3-pentone (4.3 g, 50.0 mmol), zinc chloride (6.82 g, 50.0 mmol) and xylene (100 mL) were added into a 500 mL round-bottomed flask, the mixture was refluxed at 160° C. for 18 hours. After the reaction was completed, the xylene was removed using a rotary evaporator to obtain a yellowish-brown viscous substance, the viscous substance was cooled to 25° C. and diluted with water (50 mL) and ethyl acetate (50 mL), the mixture was stirred to form suspension and filtered to give a gray white solid (6.26 g, 76.3%).

LC-MS: (M+1) m/z=330.1.

### Step 4: synthesis of 6-(3,5-difluorophenoxy)-2-ethyl-3,7-dimethylquinolin-4-yl (2-methoxyethyl)carbonate

Under N₂, 6-(3,5-difluorophenoxy)-2-ethyl-3,7-dimethylquinolin-4-ol (0.700 g, 1.6 mmol), triethylamine (1.0 mL, 7.2 mmol) and dichloromethane (10 mL) were added into a 50 mL round-bottomed flask, the mixture was cooled to 0° C. in an ice-salt bath, and then ethyl 2-methoxychloroformate (0.28 g, 2.0 mmol) was added, the resulting mixture was stirred for 12 hours. The solvent was removed using a rotary evaporator, the residue was diluted with water (30 mL) and the resulting mixture was extracted with ethyl acetate (30 mL), the organic layer was dried over magnesium sulfate and concentrated using a rotary evaporator to give a yellow liquid (0.603 g, 66.7%).

¹H NMR (400 MHz, CDCl₃) δ (ppm): 7.95 (s, 1H), 7.22 (s, 1H), 7.16-7.00 (m, 3H), 4.41 (m, 2H), 3.64 (m, 2H), 3.37 (s, 3H), 3.01 (q, 2H), 2.41 (s, 3H), 2.32 (s, 3H), 1.38 (t, 3H);

LC-MS: (M+1) m/z=432.1.

### Example 6: synthesis of 6-(2-bromophenoxy)-2-ethyl-3,7-dimethylquinolin-4-yl (2-methoxyethyl)carbonate

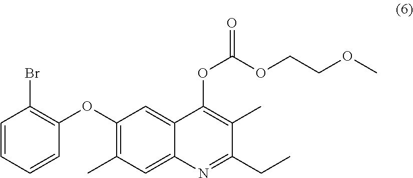 [SMILES: CCc1nc2cc(C)c(Oc3ccccc3Br)cc2c(OC(=O)OCCOC)c1C]

### Step 1: synthesis of isopropyl 5-(2-bromophenoxy)-4-methyl-2-nitrobenzoate

Isopropyl 5-fluoro-4-methyl-2-nitrobenzoate (9.60 g, 39.8 mmol), 2-bromophenol (7.12 g, 40.9 mmol) and potassium carbonate (5.52 g, 40.0 mmol) were added into a 250 mL round-bottomed flask, and then N,N-dimethylformamide (100 mL) was added, the mixture was heated to 80° C. quickly and stirred for 18 hours under N₂. To the mixture was added ice-water (100 mL), the resulting mixture was extracted with ethyl acetate (60 mL×3), the organic layers were washed with saturated aqueous sodium bicarbonate solution (50 mL×3), and then the organic layers were combined and dried over anhydrous magnesium sulfate, and then filtered, the filtrate was concentrated using a rotary evaporator to give a yellow oil (12.6 g, 80.2%).

LC-MS: (M+1) m/z=395.2.

### Step 2: Synthesis of isopropyl 2-amino-5-(2-bromophenoxy)-4-methylbenzoate

Under N₂, iron powder (5.60 g, 100.0 mmol), ethanol (100 mL), water (20 mL) and hydrochloric acid (1.0 mL, 35%) were added into a 500 mL round-bottomed flask, the mixture was heated to 90° C. and stirred, a solution of isopropyl 5-(2-bromophenoxy)-4-methyl-2-nitrobenzoate (11.5 g, 30.0 mmol) in ethanol (100 mL) was added dropwise slowly. After the addition, the mixture was stirred at 90° C. for 12 hours. The solid impurities were removed by filtration and the filtrate was concentrated using a rotary evaporator, the residue was diluted with ethyl acetate (150 mL), and the resulting solution was washed with brine (30 mL×3), the organic layers were combined and dried over anhydrous magnesium sulfate, and then filtered, the filtrate was concentrated using a rotary evaporator, the residue was purified on a silica gel column [PE/EtOAc (v/v)=10/1] to give a yellow oil (9.5 g, 89.6%).

LC-MS: (M+1) m/z=365.2.

### Step 3: synthesis of 6-(2-bromophenoxy)-2-ethyl-3,7-dimethylquinolin-4-ol

Under N₂, isopropyl 2-amino-5-(2-bromophenoxy)-4-methylbenzoate (10.0 g, 27.4 mmol), 3-pentone (4.3 g, 50.0 mmol), zinc chloride (6.82 g, 50.0 mmol) and xylene (100 mL) were added into a 500 mL round-bottomed flask, the mixture was refluxed at 160° C. for 18 hours. After the reaction was completed, the xylene was removed using a rotary evaporator to obtain a yellowish-brown viscous substance, the viscous substance was cooled to 25° C. and diluted with water (50 mL) and ethyl acetate (50 mL), the mixture was stirred to form suspension and filtered to give a gray white solid (7.83 g, 76.8%).

LC-MS: (M+1) m/z=373.2.

### Step 4: synthesis of 6-(2-bromophenoxy)-2-ethyl-3,7-dimethylquinolin-4-yl (2-methoxyethyl)carbonate

Under N₂, 6-(2-bromophenoxy)-2-ethyl-3,7-dimethylquinoline-4-ol (0.900 g, 1.6 mmol), triethylamine (1.0 mL, 7.2 mmol) and dichloromethane (10 mL) were added into a 50 mL round-bottomed flask, the mixture was cooled to 0° C. in an ice-salt bath, and then ethyl 2-methoxychloroformate (0.28 g, 2.0 mmol) was added, the resulting mixture was stirred for 12 hours. The solvent was removed using a rotary evaporator, the residue was diluted with water (30 mL) and the resulting mixture was extracted with ethyl acetate (30 mL), the organic layer was dried over magnesium sulfate and concentrated using a rotary evaporator to give a yellow liquid (0.753 g, 65.2%).

¹H NMR (400 MHz, CDCl₃) δ (ppm): 7.58 (s, 1H), 7.56 (s, 1H), 7.51 (s, 1H), 7.36 (s, 1H), 7.06-7.03 (m, 2H), 4.31 (m, 2H), 4.28 (m, 2H), 3.4 (m, 2H), 3.37 (s, 3H), 2.40 (s, 3H), 2.31 (s, 3H), 1.35 (t, 3H);

LC-MS: (M+1) m/z=475.3.

### Synthesis of Intermediates P1 to P8:

The intermediates P1 to P8 listed in table 1 can be obtained by reacting intermediate O (the structure is

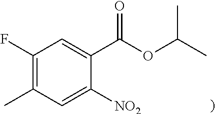 [SMILES: Cc1cc([N+](=O)[O-])c(C(=O)OC(C)C)cc1F]

with different materials respectively according to the synthetic method described in step 1 of example 1 (or example 2; or example 3; or example 4; or example 5; or example 6).

TABLE 1

| Structures and names of | | Characterization |
|---|---|---|
| the materials reacting | Structures, names and numbers of the | data of |
| with intermediate O | intermediates | intermediates |
| 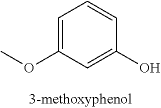 | 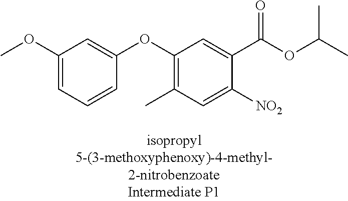 [SMILES: COc1cccc(Oc2cc(C(=O)OC(C)C)c([N+](=O)[O-])cc2C)c1] | yellow oil; LC-MS: (M + 1) m/z = 346.1. |
| 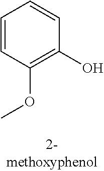 | 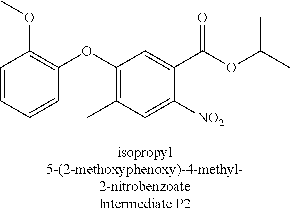 [SMILES: COc1ccccc1Oc1cc(C(=O)OC(C)C)c([N+](=O)[O-])cc1C] | yellow oil; LC-MS: (M + 1) m/z = 346.1. |
| 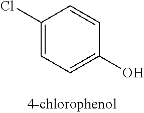 | 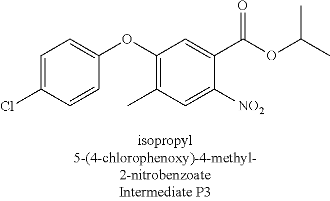 [SMILES: Cc1cc([N+](=O)[O-])c(C(=O)OC(C)C)cc1Oc1ccc(Cl)cc1] | yellow oil; LC-MS: (M + 1) m/z = 350.0. |
| 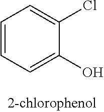 | 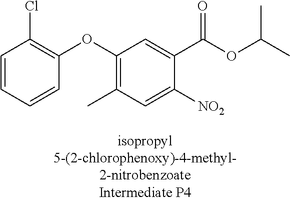 [SMILES: Cc1cc([N+](=O)[O-])c(C(=O)OC(C)C)cc1Oc1ccccc1Cl] | yellow oil; LC-MS: (M + 1) m/z = 350.0. |
| 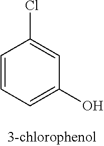 | 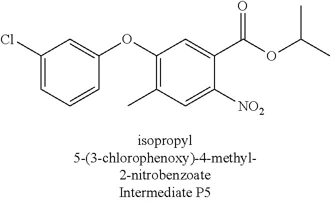 [SMILES: Cc1cc([N+](=O)[O-])c(C(=O)OC(C)C)cc1Oc1cccc(Cl)c1] | yellow oil; LC-MS: (M + 1) m/z = 350.0. |
| 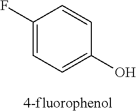 | 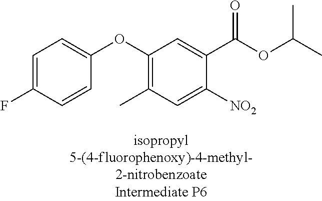 [SMILES: Cc1cc([N+](=O)[O-])c(C(=O)OC(C)C)cc1Oc1ccc(F)cc1] | yellow oil; LC-MS: (M + 1) m/z = 334.1. |
| 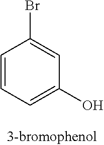 | 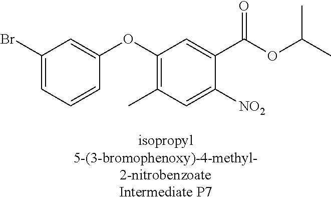 [SMILES: Cc1cc([N+](=O)[O-])c(C(=O)OC(C)C)cc1Oc1cccc(Br)c1] | yellow oil; LC-MS: (M + 1) m/z = 394.0. |
| 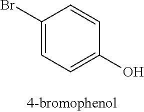 | 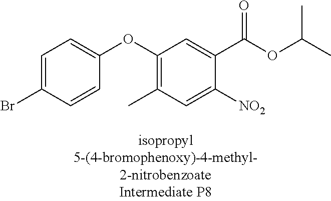 [SMILES: Cc1cc([N+](=O)[O-])c(C(=O)OC(C)C)cc1Oc1ccc(Br)cc1] | yellow oil; LC-MS: (M + 1) m/z = 394.0. |

Intermediates P1 to P8 listed in table 1 were reduced respectively by a reducer (such as iron powder, ammonium formate/palladium carbon, hydrogen/palladium carbon, etc.) to obtain the intermediates Q1 to Q8 listed in table 2.

TABLE 2

| Structures, names and numbers of the | Characterization data of |
|---|---|
| intermediates | intermediates |
| 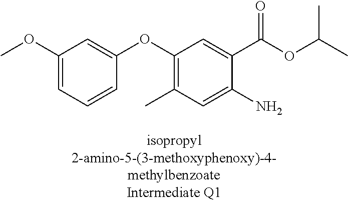 [SMILES: COc1cccc(Oc2cc(C(=O)OC(C)C)c(N)cc2C)c1] | yellow oil; LC-MS: (M + 1) m/z = 316.1. |
| 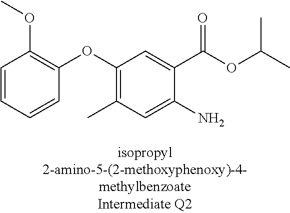 [SMILES: COc1ccccc1Oc1cc(C(=O)OC(C)C)c(N)cc1C] | yellow oil; LC-MS: (M + 1) m/z = 316.1. |
| 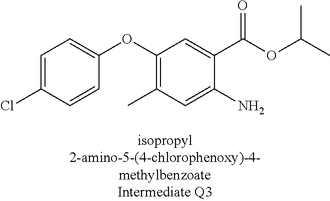 [SMILES: Cc1cc(N)c(C(=O)OC(C)C)cc1Oc1ccc(Cl)cc1] | yellow oil; LC-MS: (M + 1) m/z = 320.1. |
| 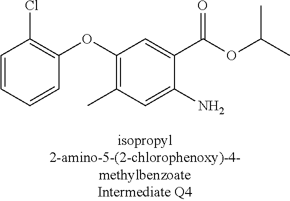 [SMILES: Cc1cc(N)c(C(=O)OC(C)C)cc1Oc1ccccc1Cl] | yellow oil; LC-MS: (M + 1) m/z = 320.1. |
| 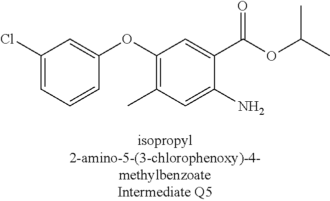 [SMILES: Cc1cc(N)c(C(=O)OC(C)C)cc1Oc1cccc(Cl)c1] | yellow oil; LC-MS: (M + 1) m/z = 320.1. |
| 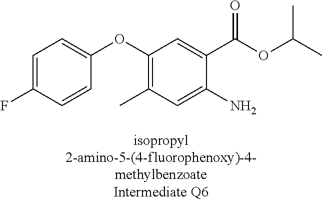 [SMILES: Cc1cc(N)c(C(=O)OC(C)C)cc1Oc1ccc(F)cc1] | yellow oil; LC-MS: (M + 1) m/z = 304.1. |
| 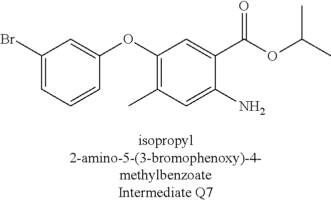 [SMILES: Cc1cc(N)c(C(=O)OC(C)C)cc1Oc1cccc(Br)c1] | yellow oil; LC-MS: (M + 1) m/z = 364.0. |
| 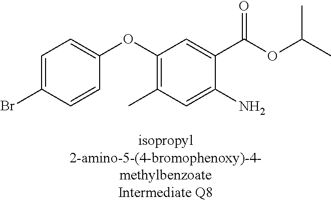 [SMILES: Cc1cc(N)c(C(=O)OC(C)C)cc1Oc1ccc(Br)cc1] | yellow oil; LC-MS: (M + 1) m/z = 364.0. |

### Synthesis of Intermediates R1 to R8:

Intermediates Q1 to Q8 listed in table 2 were reacted with 3-pentone (the structure is

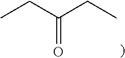

by a quinoline cyclization reaction in the present of a Lewis acid (such as zinc chloride aluminum chloride, etc.) to obtain the intermediates R1 to R8 listed in table 3.

TABLE 3

| | Characterization data of |
|---|---|
| Structures, names and numbers of the intermediates | intermediates |
| 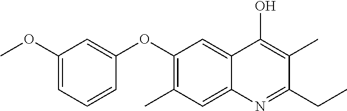 [SMILES: CCc1nc2cc(C)c(Oc3cccc(OC)c3)cc2c(O)c1C] | gray white solid; LC-MS: (M + 1) m/z = 324.1. |
| 2-ethyl-6-(3-methoxyphenoxy)-3,7-dimethylquinolin-4-ol | |
| Intermediate R1 | |
| 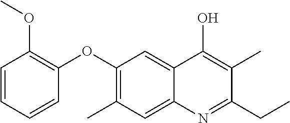 [SMILES: CCc1nc2cc(C)c(Oc3ccccc3OC)cc2c(O)c1C] | gray white solid LC-MS: (M + 1) m/z = 324.1. |
| 2-ethyl-6-(2-methoxyphenoxy)-3,7-dimethylquinolin-4-ol | |
| Intermediate R2 | |
| 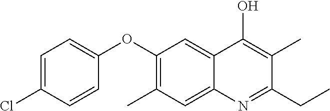 [SMILES: CCc1nc2cc(C)c(Oc3ccc(Cl)cc3)cc2c(O)c1C] | gray white solid LC-MS: (M + 1) m/z = 328.1. |
| 6-(4-chlorophenoxy)-2-ethyl-3,7-dimethylquinolin-4-ol | |
| Intermediate R3 | |
| 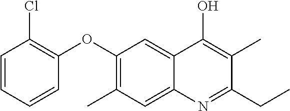 [SMILES: CCc1nc2cc(C)c(Oc3ccccc3Cl)cc2c(O)c1C] | gray white solid; LC-MS: (M + 1) m/z = 328.1. |
| 6-(2-chlorophenoxy)-2-ethyl-3,7-dimethylquinolin-4-ol | |
| Intermediate R4 | |
| 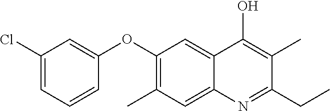 [SMILES: CCc1nc2cc(C)c(Oc3cccc(Cl)c3)cc2c(O)c1C] | gray white solid LC-MS: (M + 1) m/z = 328.1. |
| 6-(3-chlorophenoxy)-2-ethy1-3,7-dimethylquinolin-4-ol | |
| Intermediate R5 | |
| 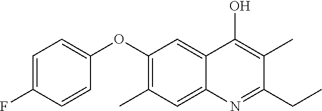 [SMILES: CCc1nc2cc(C)c(Oc3ccc(F)cc3)cc2c(O)c1C] | gray white solid; LC-MS: (M + 1) m/z = 312.1. |
| 6-(4-fluorophenoxy)-2-ethyl-3,7-dimethylquinolin-4-ol | |
| Intermediate R6 | |
| 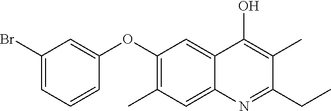 [SMILES: CCc1nc2cc(C)c(Oc3cccc(Br)c3)cc2c(O)c1C] | gray white solid; LC-MS: (M + 1) m/z = 372.0. |
| 6-(3-bromophenoxy)-2-ethyl-3,7-dimethylquinolin-4-ol | |
| Intermediate R7 | |
| 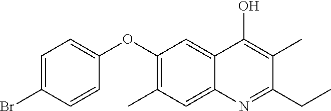 [SMILES: CCc1nc2cc(C)c(Oc3ccc(Br)cc3)cc2c(O)c1C] | gray white solid; LC-MS: (M + 1) m/z = 372.0. |
| 6-(4-bromophenoxy)-2-ethyl-3,7-dimethylquinolin-4-ol | |
| Intermediate R8 | |

### Synthesis of Target Compounds of Examples 7 to 17

Intermediates R1 to R8 listed in table 3 respectively with 2-ethyl-3,7-dimethyl-6-(4-(trifluoromethoxy)phenoxy)-quinolin-4-ol (the intermediate prepared by step 3 of example 1 having structure

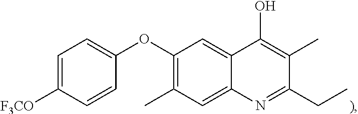 [SMILES: CCc1nc2cc(C)c(Oc3ccc(OC(F)(F)F)cc3)cc2c(O)c1C]

were reacted with 2-(2-methoxyethoxy)ethyl chloroformate (the structure is

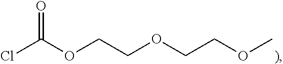

or with (tetrahydrofuran-2-yl)methyl chloroformate (the structure is

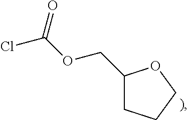

or with ethyl 2-methoxy chloroformate (the structure is

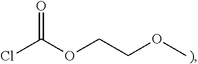

or with ethyl 2-ethoxychloroformate

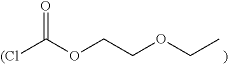

under a base (such as pyridine, sodium hydride, triethylamine, etc.) to obtain the target compounds of examples 7 to 17 listed in table 4.

TABLE 4

| | Structures, names of the target | |
|---|---|---|
| | compounds and numbers of the | Characterization data of the |
| Reaction substrates | examples | target compounds |
| 2-ethyl-3,7-dimethyl-6-(4- (trifluoromethoxy)phenoxy) quinolin-4-ol and 2-(2-methoxyethoxy)ethyl chloroformate | 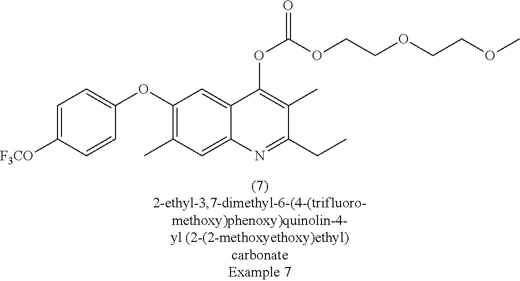 [SMILES: CCc1nc2cc(C)c(Oc3ccc(OC(F)(F)F)cc3)cc2c(OC(=O)OCCOCCOC)c1C] | yellow liquid; ¹H NMR(400 MHz, CDCl₃) δ (ppm): 7.98 (s, 1H), 7.28 (s, 1H), 7.17 (s, 1H), 6.99 (s, 1H), 6.98 (s, 1H), 6.83 (s, 1H), 4.29 (t, 2H), 3.67 (t, 2H), 3.56 (s, 4H), 3.38 (t, 2H), 3.32 (s, 3H), 2.36 (s, 3H), 2.13 (s, 3H), 1.29 (t, 3H); LC-MS: (M + 1) m/z = 524.5. |
| 2-ethyl-3,7-dimethyl-6-(4- (trifluoromethoxy)phenoxy) quinolin-4-ol and (tetrahydrofuran-2-yl)meth- yl chloroformate | 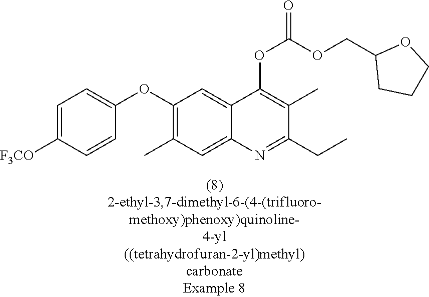 [SMILES: CCc1nc2cc(C)c(Oc3ccc(OC(F)(F)F)cc3)cc2c(OC(=O)OCC2CCCO2)c1C] | yellow liquid; ¹H NMR(400 MHz, CDCl₃) δ (ppm): 7.83 (s, 1H), 7.22 (s, 1H), 7.16 (s, 1H), 6.95 (s, 1H), 6.93 (s, 1H), 6.85 (s, 1H), 4.46 (m, 2H), 3.37 (m, 1H), 3.86 (m, 2H), 3.48 (t, 2H), 2.56 (s, 3H), 2.18 (s, 3H), 1.90-1.86 (m, 2H), 1.68-1.65 (m, 2H), 1.25 (t, 3H); LC-MS: (M + 1) m/z = 506.4. |
| Intermediate R1 and ethyl 2-methoxychloroformate | 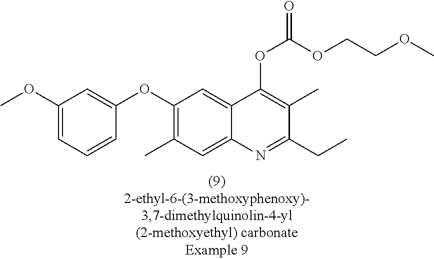 | yellow liquid; ¹H NMR (400 MHz, CDCl3) δ (ppm): 7.93 (s, 1H), 7.49 (s, 1H), 7.32-7.30 (m, 1H), 7.22 (s, 1H), 6.73- 6.69 (m, 2H), 4.42 (t, 2H), 3.74 (s, 3H), 3.66 (t, 2H), 3.38 (s, 3H), 3.03 (q, 2H), 2.41 (s, 3H), 2.31 (s, 3H), 1.38 (t, 3H); LC-MS: (M + 1) m/z = 426.1. |
| Intermediate R2 and ethyl 2-methoxychloroformate | 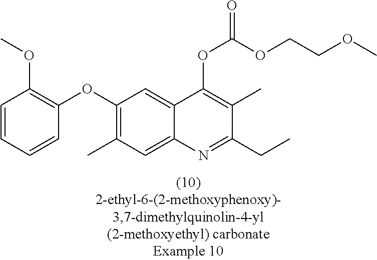 [SMILES: CCc1nc2cc(C)c(Oc3ccccc3OC)cc2c(OC(=O)OCCOC)c1C] | yellow liquid; ¹H NMR (400 MHz, CDCl3) δ (ppm): 7.94 (s, 1H), 7.32-7.29 (m, 1H), 7.23 (s, 1H), 6.73-6.69 (m, 2H), 6.74 (d, 1H), 4.41 (t, 2H), 3.72 (s, 3H), 3.64 (t, 2H), 3.41 (s, 3H), 3.03 (q, 2H), 2.41 (s, 3H), 2.32 (s, 3H), 1.38 (t, 3H); LC-MS: (M + 1) m/z = 426.1. |
| Intermediate R3 and ethyl 2-methoxychloroformate | 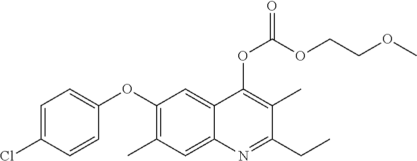 [SMILES: CCc1nc2cc(C)c(Oc3ccc(Cl)cc3)cc2c(OC(=O)OCCOC)c1C] | yellow liquid; ¹H NMR (400 MHz, CDCl3) δ (ppm): 7.94 (s, 1H), 7.55 (d, 2H), 7.45 (d, 2H), 7.23 (s, 1H), 4.41 (t, 2H), 3.62 (t, 2H), 3.42 (s, 3H), 3.03 (q, 2H), 2.40 (s, 3H), 2.32 (s, 3H), 1.38 (t, 3H); LC-MS: (M + 1) m/z = 430.1. |
| | (11) | |
| | 6-(4-chlorophenoxy)-2-ethyl-3,7- | |
| | dimethylquinolin-4-yl | |
| | (2-methoxyethyl) carbonate | |
| | Example 11 | |
| Intermediate R4 and 2-methoxychloroformate ethyl | 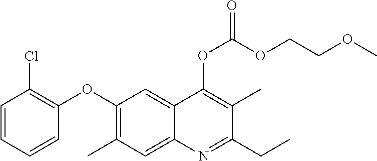 [SMILES: CCc1nc2cc(C)c(Oc3ccccc3Cl)cc2c(OC(=O)OCCOC)c1C] | yellow liquid; ¹H NMR (400 MHz, CDCl₃) δ (ppm): 7.92 (s, 1H), 7.54 (s, 1H), 7.48 (t, 1H), 7.31-7.28 (m, 1H), 7.23- 7.18 (m, 2H), 4.40 (t, 2H), 3.62 (t, 2H), 3.41(s, 3H), 3.03 (q, 2H), 2.42 (s, 3H), 2.32 (s, 3H), 1.38 (t, 3H); LC-MS: (M + 1) m/z = 430.1. |
| | (12) | |
| | 6-(2-chlorophenoxy)-2-ethyl-3,7- | |
| | dimethylquinolin-4-yl | |
| | (2-methoxyethyl) carbonate | |
| | Example 12 | |
| Intermediate R5 and ethyl 2-methoxychloroformate | 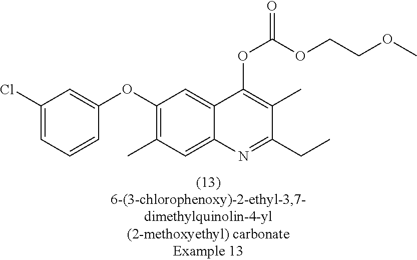 [SMILES: CCc1nc2cc(C)c(Oc3cccc(Cl)c3)cc2c(OC(=O)OCCOC)c1C] | yellow liquid; ¹H NMR (400 MHz, CDCl₃) δ (ppm): 7.94 (s, 1H), 7.53 (s, 1H), 7.36-7.33 (m, 2H), 7.24-7.21 (m, 1H), 7.02-7.00 (m, 1H), 4.40 (t, 2H), 3.63 (t, 2H), 3.40 (s, 3H), 3.02 (q, 2H), 2.41 (s, 3H), 2.32 (s, 3H), 1.38 (t, 3H); LC-MS: (M + 1) m/z = 430.1. |
| Intermediate R6 and ethyl 2-methoxychloroformate | 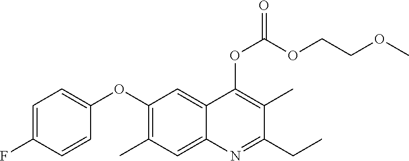 [SMILES: CCc1nc2cc(C)c(Oc3ccc(F)cc3)cc2c(OC(=O)OCCOC)c1C] | yellow liquid; ¹H NMR (400 MHz, CDCl₃) δ (ppm): 7.95 (s, 1H), 7.52 (d, 2H), 7.42 (d, 2H), 7.22 (s, 1H), 4.41 (t, 2H), 3.63 (t, 2H), 3.41 (s, 3H), 3.03 (q, 2H), 2.42 (s, 3H), 2.31 (s, 3H), 1.39 (t, 3H); LC-MS: (M + 1) m/z = 414.1. |
| | (14) | |
| | 2-ethyl-6-(4-fluorophenoxy)-3,7- | |
| | dimethylquinolin-4-yl | |
| | (2-methoxyethyl) carbonate | |
| | Example 14 | |
| Intermediate R7 and ethyl 2-methoxychloroformate | 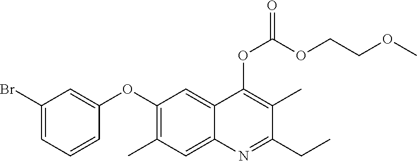 [SMILES: CCc1nc2cc(C)c(Oc3cccc(Br)c3)cc2c(OC(=O)OCCOC)c1C] | yellow liquid; ¹H NMR (400 MHz, CDCl₃) δ (ppm): 7.93 (s, 1H), 7.58 (s, 1H), 7.41 (d, 1H), 7.28-7.22 (m, 2H), 7.10 (d, 1H), 4.40 (t, 2H), 3.63 (t, 2H), 3.41 (s, 3H), 3.01 (q, 2H), 2.42 (s, 3H), 2.32 (s, 3H), 1.38 (t, 3H); LC-MS: (M + 1) m/z = 474.0. |
| | (15) | |
| | 6-(3-bromophenoxy)-2-ethyl-3,7- | |
| | dimethylquinolin-4-yl | |
| | (2-methoxyethyl) carbonate | |
| | Example 15 | |
| Intermediate R8 and ethyl 2-methoxychloroformate | 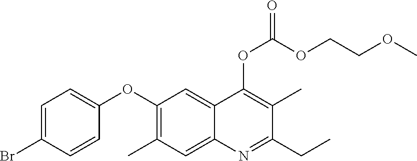 [SMILES: CCc1nc2cc(C)c(Oc3ccc(Br)cc3)cc2c(OC(=O)OCCOC)c1C] | yellow liquid; ¹H NMR (400 MHz, CDCl₃) δ (ppm): 7.95 (s, 1H), 7.60 (d, 2H), 7.41 (d, 2H), 7.22 (s, 1H), 4.42 (t, 2H), 3.63 (t, 2H), 3.41 (s, 3H), 3.02 (q, 2H), 2.42 (s, 3H), 2.32 (s, 3H), 1.37 (t, 3H); LC-MS: (M + 1) m/z = 474.0. |
| | (16) | |
| | 6-(4-bromophenoxy)-2-ethyl-3,7- | |
| | dimethylquinolin-4-yl | |
| | (2-methoxyethyl) carbonate | |
| | Example 16 | |
| 2-ethyl-3,7-dimethyl-6-(4- (trifluoromethoxy)phenoxy) quinolin-4-ol and ethyl 2-ethoxychloroformate | 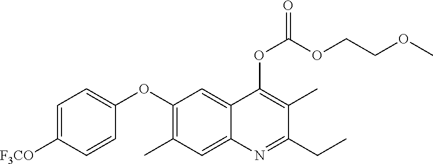 | yellow liquid; ¹H NMR (400 MHz, CDCl3) δ (ppm): 7.59 (s, 1H), 7.54 (s, 1H), 7.39 (d, 2H), 6.97 (d, 2H), 4.48 (t, 2H), 3.69 (t, 2H), 3.59 (q, 2H), 3.42 (q, 2H), 2.32 (s, 3H), 2.13 (s, 3H), 1.28 (t, 3H), 1.16 (t, 3H); LC-MS: (M + 1) m/z = 494.1. |
| | (17) | |
| | 2-ethoxyethyl | |
| | (2-ethyl-3,7-dimethyl-6-(4-(trifluoro- | |
| | methoxy)phenoxy)quinolin-4- | |
| | yl) carbonate | |
| | Example 17 | |

### Example 18: Synthesis of 2-ethyl-3,5,7-trimethyl-6-(4-(trifluoromethoxy)phenoxy)quinolin-4-yl (2-methoxyethyl) carbonate

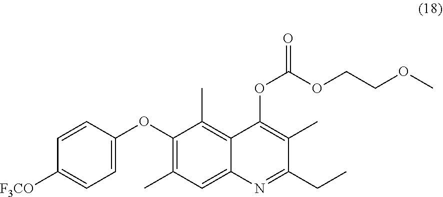 [SMILES: CCc1nc2cc(C)c(Oc3ccc(OC(F)(F)F)cc3)c(C)c2c(OC(=O)OCCOC)c1C]

### Step 1: Synthesis of 1,3-dimethyl-5-nitro-2-(4-(trifluoromethoxy)phenoxy)benzene

2-Fluoro-1,3-methyl-5-nitrobenzene (10.0 g, 59.2 mmol), p-trifluoromethoxyphenol (15.8 g, 88.7 mmol) and potassium carbonate (12.2 g, 88.7 mmol) were added into a 250 mL round-bottomed flask, and then N,N-dimethylformamide (100 mL) was added, the mixture was heated to 110° C. quickly and stirred for 24 hours under N₂. To the mixture was added ice-water (100 mL), the resulting mixture was extracted with ethyl acetate (60 mL×3), the organic layers were washed with saturated aqueous sodium bicarbonate solution (50 mL×3), and then the organic layers were combined and dried over anhydrous magnesium sulfate, and then filtered, the filtrate was concentrated using a rotary evaporator to give a yellow oil (14.4 g, 75%).

LC-MS: (M+1) m/z=328.2.

### Step 2: synthesis of 3,5-dimethyl-4-(4-(trifluoromethoxy)phenoxy)aniline

Under N₂, iron powder (6.2 g, 110.1 mmol), ethanol (100 mL), water (20 mL) and hydrochloric acid (1.0 mL, 35%) were added into a 500 mL round-bottomed flask, the mixture was heated to 90° C. and stirred, a solution of 1,3-dimethyl-5-nitro-2-(4-(trifluoromethoxy)phenoxy)benzene (12.0 g, 36.7 mmol) in ethanol (100 mL) was added dropwise slowly. After the addition, the mixture was stirred at 90° C. for 2 hours. The solid impurities were removed by filtration and the filtrate was concentrated using a rotary evaporator, the residue was diluted with ethyl acetate (150 mL), and the resulting mixture was washed with saturated aqueous NaCl (30 mL×3), the organic layers were combined and dried over anhydrous magnesium sulfate, and then filtered, the filtrate was concentrated using a rotary evaporator, the residue was purified on a silica gel column eluted with PE/EtOAc (V/V)=10/1 to give a yellow oil (10.0 g, 92%).

LC-MS: (M+1) m/z=298.2.

### Step 3: synthesis of 2-ethyl-3,5,7-trimethyl-6-(4-(trifluoromethoxy)phenoxy)quinolin-4-ol (compound 42)

3,5-Dimethyl-4-(4-(trifluoromethoxy)phenoxy)aniline (10 g, 33.7 mmol), ethyl 2-methyl-3-oxovalerate (15.9 g, 101 mmol), glacial acetic acid (0.1 g, 1.67 mmol) and toluene (100 mL) were added into a 100 mL single neck flask equipped with a water separator under N₂, the mixture was stirred at 120° C. and refluxed for 18 hours to separate water. The toluene was evaporated to obtain an oil; then diphenyl ether (20 mL) was added into the oil, the mixture was heated to 255° C. and stirred for 30 min. The mixture was cooled to rt, and PE (100 mL) was added slowly to precipitate the solid. The precipitated solids were collected by filtration, washed with PE/EtOAc (v/v=20/1, 10 mL×3) to get a grayish brown solid (5.9 g, 45%).

LC-MS: (M+1) m/z=392.4.

### Step 4: synthesis of 2-ethyl-3,5,7-trimethyl-6-(4-(trifluoromethoxy)phenoxy)quinolin-4-yl (2-methoxyethyl) carbonate

Under N₂, 2-ethyl-3,5,7-trimethyl-6-(4-(trifluoromethoxy)phenoxy)quinolin-4-ol (0.80 g, 2.0 mmol), triethylamine (1.0 mL, 6.0 mmol) and dichloromethane (10 mL) were added into a 50 mL round-bottomed flask, the mixture was cooled to 0° C. in an ice-salt bath, and then ethyl 2-methoxychloroformate (0.37 g, 2.7 mmol) was added, the resulting mixture was stirred at rt for 12 hours. The solvent was removed using a rotary evaporator, the residue was diluted with water (30 mL) and ethyl acetate (30 mL), the organic layer was dried over magnesium sulfate and concentrated using a rotary evaporator to give a yellow liquid (0.74 g, 75%).

¹H NMR (400 MHz, CDCl₃) δ (ppm): 7.47 (s, 1H), 7.38 (s, 2H), 6.99 (s, 2H), 4.35-4.32 (m, 2H), 3.67-3.64 (m, 2H), 3.43 (q, J=7.5 Hz, 2H), 3.34 (s, 3H), 2.34 (s, 3H), 2.31 (s, 3H), 2.14 (s, 3H), 1.28 (t, J=7.5 Hz, 3H);

LC-MS: (M+1) m/z=494.4.

### Synthesis of Intermediates S1 to S17:

A phenol derivative was reacted with 1-fluoro-2-methyl-4-nitrobenzene (the structure is

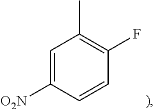

or with 2-fluoro-1,3-dimethyl-5-nitrobenzene (the structure is

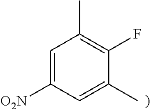

under a base (potassium carbonate, cesium carbonate, sodium hydride, sodium hydroxide, etc.) to obtain intermediates S1 to S17 listed in table 5.

TABLE 5

| | | Characterization |
|---|---|---|
| Structures and names of | Structures, names and numbers of the | data of |
| reaction substrates | intermediates | intermediates |
| 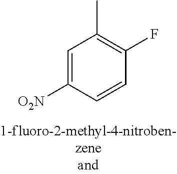 | 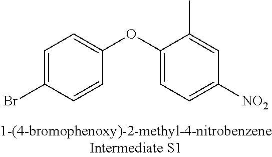 [SMILES: Cc1cc([N+](=O)[O-])ccc1Oc1ccc(Br)cc1] | yellow solid; LC-MS: (M + 1) m/z = 308.0. |
| 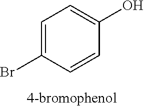 | | |
| 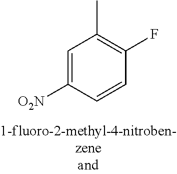 | 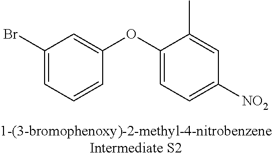 [SMILES: Cc1cc([N+](=O)[O-])ccc1Oc1cccc(Br)c1] | yellow solid; LC-MS: (M + 1) m/z = 308.0 |
| 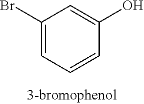 | | |
| 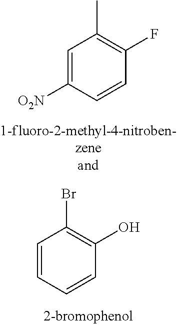 | 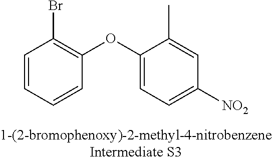 [SMILES: Cc1cc([N+](=O)[O-])ccc1Oc1ccccc1Br] | yellow solid; LC-MS: (M + 1) m/z = 308.0 |
| 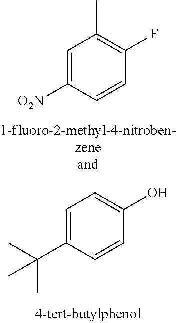 | 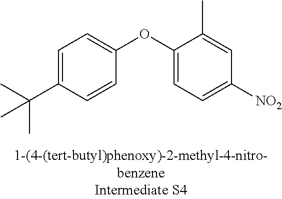 [SMILES: Cc1cc([N+](=O)[O-])ccc1Oc1ccc(C(C)(C)C)cc1] | yellow solid; LC-MS: (M + 1) m/z = 286.1. |
| 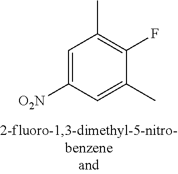 | 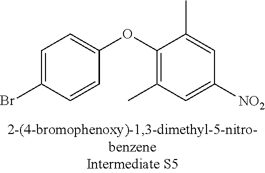 [SMILES: Cc1cc([N+](=O)[O-])cc(C)c1Oc1ccc(Br)cc1] | yellow solid; LC-MS: (M + 1) m/z = 322.0. |
| 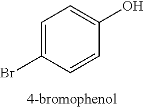 | | |
| 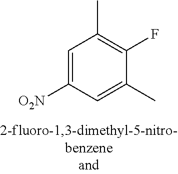 | 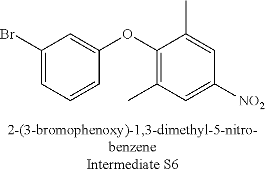 [SMILES: Cc1cc([N+](=O)[O-])cc(C)c1Oc1cccc(Br)c1] | yellow solid; LC-MS: (M + 1) m/z = 322.0. |
| 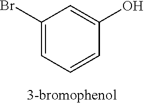 | | |
| 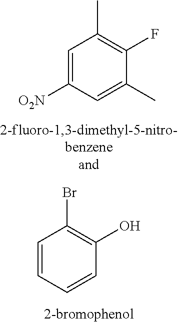 | 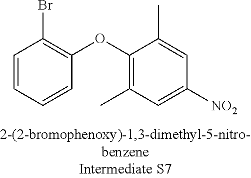 [SMILES: Cc1cc([N+](=O)[O-])cc(C)c1Oc1ccccc1Br] | yellow solid; LC-MS: (M + 1) m/z = 322.0. |
| 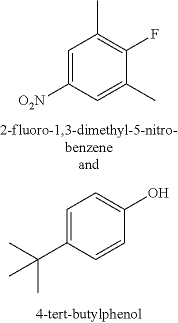 | 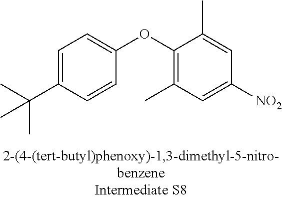 [SMILES: Cc1cc([N+](=O)[O-])cc(C)c1Oc1ccc(C(C)(C)C)cc1] | yellow solid; LC-MS: (M + 1) m/z = 300.2. |
| 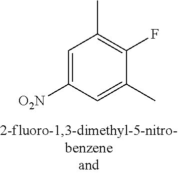 | 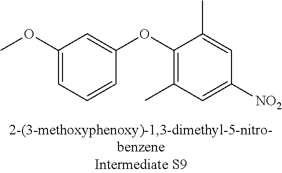 [SMILES: COc1cccc(Oc2c(C)cc([N+](=O)[O-])cc2C)c1] | yellow solid; LC-MS: (M + 1) m/z = 274.1. |
| 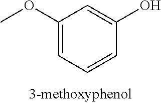 | | |
| 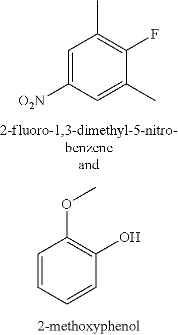 | 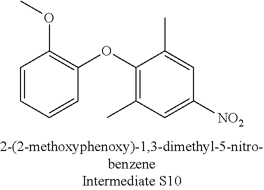 [SMILES: COc1ccccc1Oc1c(C)cc([N+](=O)[O-])cc1C] | yellow solid; LC-MS: (M + 1) m/z = 274.1. |
| 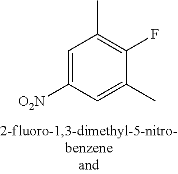 | 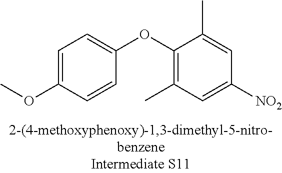 [SMILES: COc1ccc(Oc2c(C)cc([N+](=O)[O-])cc2C)cc1] | yellow solid; LC-MS: (M + 1) m/z = 274.1. |
| 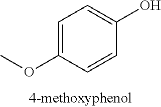 | | |
| 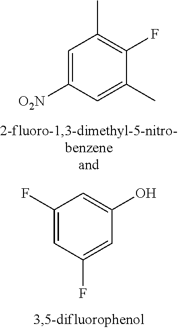 | 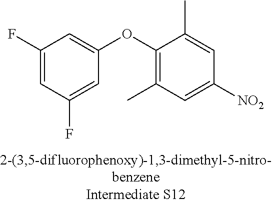 [SMILES: Cc1cc([N+](=O)[O-])cc(C)c1Oc1cc(F)cc(F)c1] | yellow solid; LC-MS: (M + 1) m/z = 280.1. |
| 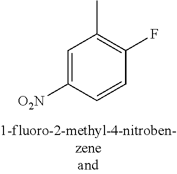 | 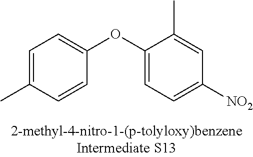 [SMILES: Cc1ccc(Oc2ccc([N+](=O)[O-])cc2C)cc1] | yellow solid; LC-MS: (M + 1) m/z = 244.1. |
| 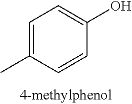 | | |
| 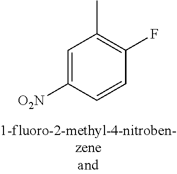 | 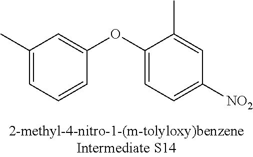 [SMILES: Cc1cccc(Oc2ccc([N+](=O)[O-])cc2C)c1] | yellow solid; LC-MS: (M + 1) m/z = 244.1. |
| 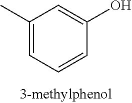 | | |
| 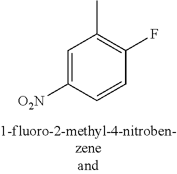 | 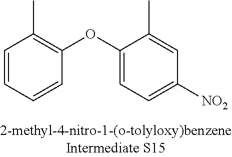 [SMILES: Cc1ccccc1Oc1ccc([N+](=O)[O-])cc1C] | yellow solid; LC-MS: (M + 1) m/z = 244.1. |
| 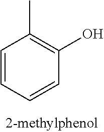 | | |
| 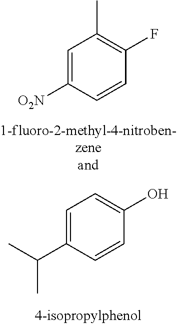 | 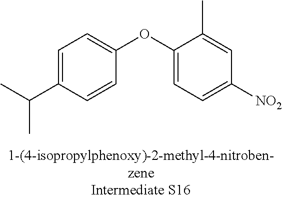 [SMILES: Cc1cc([N+](=O)[O-])ccc1Oc1ccc(C(C)C)cc1] | yellow solid; LC-MS: (M + 1) m/z = 272.1. |
| 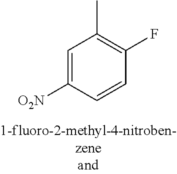 | 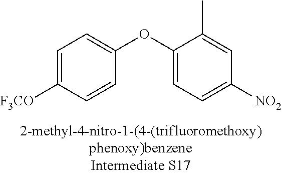 [SMILES: Cc1cc([N+](=O)[O-])ccc1Oc1ccc(OC(F)(F)F)cc1] | yellow solid; LC-MS: (M + 1) m/z = 314.0. |
| 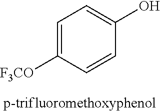 [SMILES: Oc1ccc(OC(F)(F)F)cc1] | | |

### Synthesis of Intermediates T1 to T17:

Intermediates S to S17 listed in table 5 were reduced respectively by a reducer (such as iron powder, ammonium formate/palladium carbon, hydrogen/palladium carbon, etc.) to obtain the intermediates T1 to T17 listed in table 6.

TABLE 6

| Structures, names and numbers of the | Characterization data |
|---|---|
| intermediates | of intermediates |
| 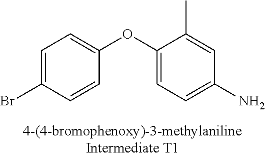 [SMILES: Cc1cc(N)ccc1Oc1ccc(Br)cc1] | yellow solid; LC-MS: (M + 1) m/z = 278.0. |
| 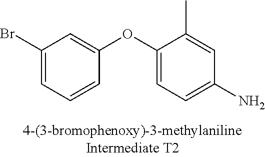 [SMILES: Cc1cc(N)ccc1Oc1cccc(Br)c1] | yellow solid; LC-MS: (M + 1) m/z = 278.0. |
| 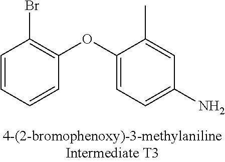 [SMILES: Cc1cc(N)ccc1Oc1ccccc1Br] | light yellow solid; LC-MS: (M + 1) m/z = 278.0. |
| 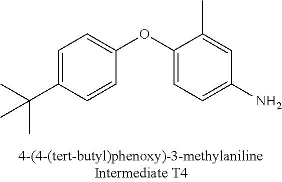 [SMILES: Cc1cc(N)ccc1Oc1ccc(C(C)(C)C)cc1] | light yellow solid; LC-MS: (M + 1) m/z = 256.2. |
| 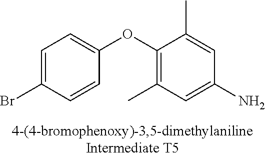 [SMILES: Cc1cc(N)cc(C)c1Oc1ccc(Br)cc1] | light yellow solid; LC-MS: (M + 1) m/z = 292.0. |
| 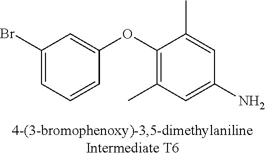 [SMILES: Cc1cc(N)cc(C)c1Oc1cccc(Br)c1] | light yellow solid; LC-MS: (M + 1) m/z = 292.0. |
| 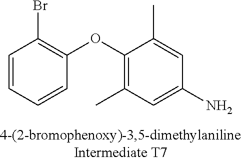 [SMILES: Cc1cc(N)cc(C)c1Oc1ccccc1Br] | light yellow solid; LC-MS: (M + 1) m/z = 292.0. |
| 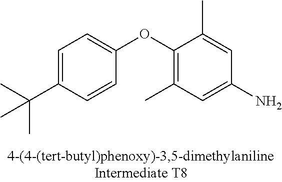 [SMILES: Cc1cc(N)cc(C)c1Oc1ccc(C(C)(C)C)cc1] | light yellow solid; LC-MS: (M + 1) m/z = 270.2. |
| 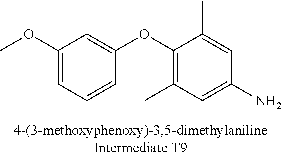 [SMILES: COc1cccc(Oc2c(C)cc(N)cc2C)c1] | light yellow solid; LC-MS: (M + 1) m/z = 244.1. |
| 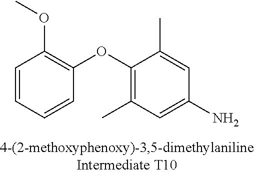 [SMILES: COc1ccccc1Oc1c(C)cc(N)cc1C] | light yellow solid; LC-MS: (M + 1) m/z = 244.1. |
| 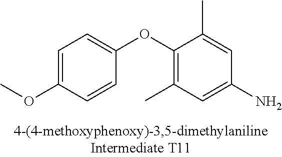 [SMILES: COc1ccc(Oc2c(C)cc(N)cc2C)cc1] | light yellow solid; LC-MS: (M + 1) m/z = 244.1. |
| 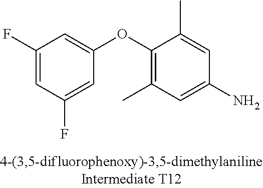 [SMILES: Cc1cc(N)cc(C)c1Oc1cc(F)cc(F)c1] | light yellow solid; LC-MS: (M + 1) m/z = 250.1. |
| 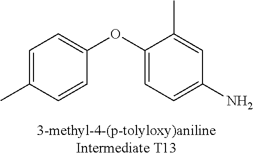 [SMILES: Cc1ccc(Oc2ccc(N)cc2C)cc1] | light yellow solid; LC-MS: (M + 1) m/z = 214.1. |
| 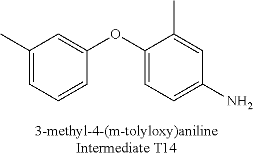 [SMILES: Cc1cccc(Oc2ccc(N)cc2C)c1] | light yellow solid; LC-MS: (M + 1) m/z = 214.1. |
| 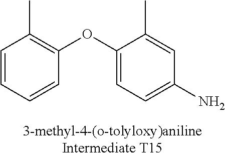 [SMILES: Cc1ccccc1Oc1ccc(N)cc1C] | light yellow solid; LC-MS: (M + 1) m/z = 214.1. |
| 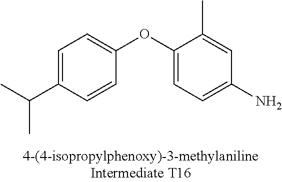 [SMILES: Cc1cc(N)ccc1Oc1ccc(C(C)C)cc1] | light yellow solid; LC-MS: (M + 1) m/z = 242.2. |
| 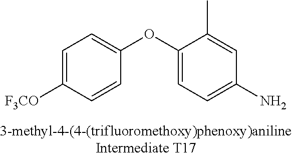 [SMILES: Cc1cc(N)ccc1Oc1ccc(OC(F)(F)F)cc1] | light yellow solid; LC-MS: (M + 1) m/z = 284.0. |

### Synthesis of Intermediates U1 to U17:

Intermediates T1 to T17 listed in table 6 were respectively reacted with ethyl 2-methyl-3-oxovalerate (the structure is

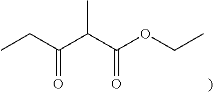

according to the synthetic method similar to those described in step 3 of example 18 to obtain the intermediates U1 to U17 listed in table 7.

TABLE 7

| | Characterization data of |
|---|---|
| Structures, names and numbers of the intermediates | intermediates |
| 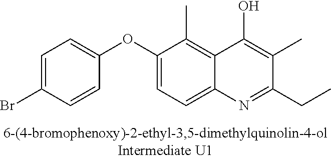 [SMILES: CCc1nc2ccc(Oc3ccc(Br)cc3)c(C)c2c(O)c1C] | brown solid; LC-MS: (M + 1) m/z = 372.0. |
| 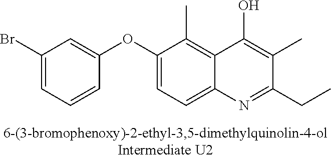 [SMILES: CCc1nc2ccc(Oc3cccc(Br)c3)c(C)c2c(O)c1C] | brown solid; LC-MS: (M + 1) m/z = 372.0. |
| 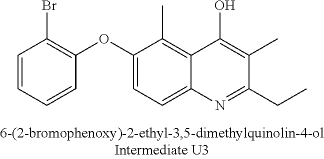 [SMILES: CCc1nc2ccc(Oc3ccccc3Br)c(C)c2c(O)c1C] | brown solid; LC-MS: (M + 1) m/z = 372.0. |
| 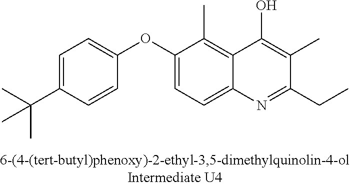 [SMILES: CCc1nc2ccc(Oc3ccc(C(C)(C)C)cc3)c(C)c2c(O)c1C] | brown solid; LC-MS: (M + 1) m/z = 350.2. |
| 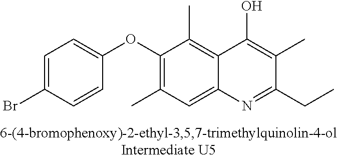 [SMILES: CCc1nc2cc(C)c(Oc3ccc(Br)cc3)c(C)c2c(O)c1C] | brown solid; LC-MS: (M + 1) m/z = 386.1. |
| 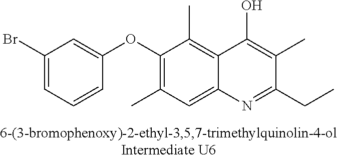 [SMILES: CCc1nc2cc(C)c(Oc3cccc(Br)c3)c(C)c2c(O)c1C] | brown solid; LC-MS: (M + 1) m/z = 386.1. |
| 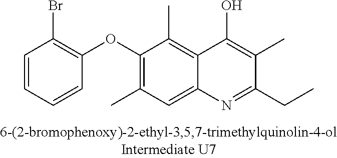 [SMILES: CCc1nc2cc(C)c(Oc3ccccc3Br)c(C)c2c(O)c1C] | brown solid; LC-MS: (M + 1) m/z = 386.1. |
| 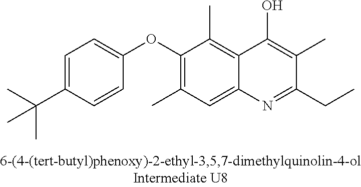 [SMILES: CCc1nc2cc(C)c(Oc3ccc(C(C)(C)C)cc3)c(C)c2c(O)c1C] | brown solid; LC-MS: (M + 1) m/z = 364.2. |
| 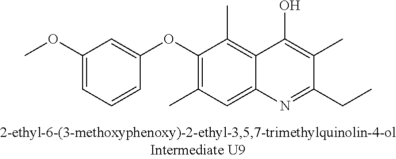 [SMILES: CCc1nc2cc(C)c(Oc3cccc(OC)c3)c(C)c2c(O)c1C] | brown solid; LC-MS: (M + 1) m/z = 338.2. |
| 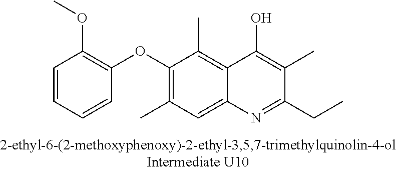 [SMILES: CCc1nc2cc(C)c(Oc3ccccc3OC)c(C)c2c(O)c1C] | brown solid; LC-MS: (M + 1) m/z = 338.2. |
| 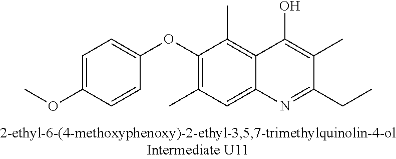 [SMILES: CCc1nc2cc(C)c(Oc3ccc(OC)cc3)c(C)c2c(O)c1C] | brown solid; LC-MS: (M + 1) m/z = 338.2. |
| 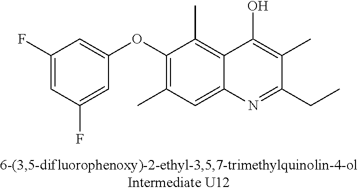 [SMILES: CCc1nc2cc(C)c(Oc3cc(F)cc(F)c3)c(C)c2c(O)c1C] | brown solid; LC-MS: (M + 1) m/z = 344.1. |
| 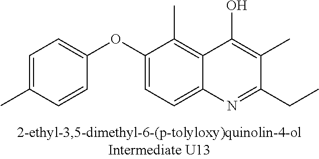 [SMILES: CCc1nc2ccc(Oc3ccc(C)cc3)c(C)c2c(O)c1C] | brown solid; LC-MS: (M + 1) m/z = 308.2. |
| 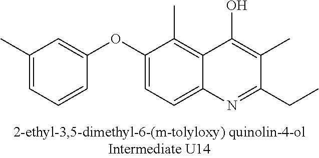 [SMILES: CCc1nc2ccc(Oc3cccc(C)c3)c(C)c2c(O)c1C] | brown solid; LC-MS: (M + 1) m/z = 308.2. |
| 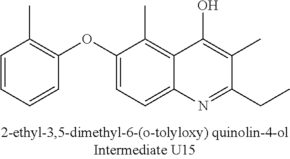 [SMILES: CCc1nc2ccc(Oc3ccccc3C)c(C)c2c(O)c1C] | brown solid; LC-MS: (M + 1) m/z = 308.2. |
| 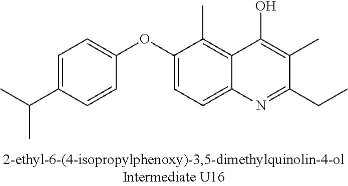 [SMILES: CCc1nc2ccc(Oc3ccc(C(C)C)cc3)c(C)c2c(O)c1C] | brown solid; LC-MS: (M + 1) m/z = 336.2. |
| 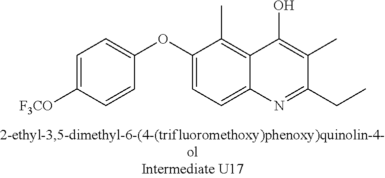 [SMILES: CCc1nc2ccc(Oc3ccc(OC(F)(F)F)cc3)c(C)c2c(O)c1C] | brown solid; LC-MS: (M + 1) m/z = 378.1. |

### Synthesis of Target Compounds of Examples 19 to 41

Intermediates U1 to U17 listed in table 7 respectively with 2-ethyl-3,5,7-trimethyl-6-(4-(trifluoromethoxy)phenoxy)-quinolin-4-ol (i.e. compound (42) prepared by step 3 of example 18 having structure

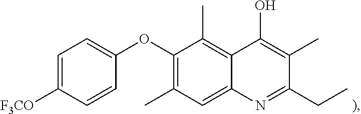 [SMILES: CCc1nc2cc(C)c(Oc3ccc(OC(F)(F)F)cc3)c(C)c2c(O)c1C]

were reacted with ethyl 2-methoxychloroformate (the structure is

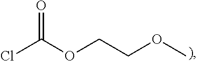

or with (tetrahydrofuran-2-yl)methyl chloroformate (the structure

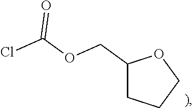

or with ethyl 2-ethoxy chloroformate (the structure is

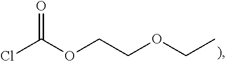

or with ethyl 2-isopropoxychloroformate (the structure is

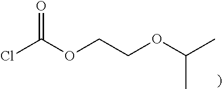

according to the synthetic method similar to those described in step 4 of example 18 to obtain the target compounds of examples 19 to 41 listed in table 8.

TABLE 8

| | Structures, names, numbers | Characterization data of the |
|---|---|---|
| Reaction substrates | of the target compounds | target compounds |
| Intermediate U1 and ethyl 2-methoxychlorofor- mate | 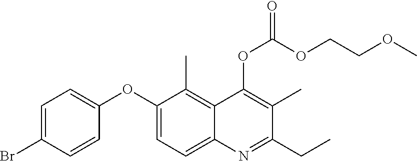 [SMILES: CCc1nc2ccc(Oc3ccc(Br)cc3)c(C)c2c(OC(=O)OCCOC)c1C] | yellow liquid; ¹H NMR (400 MHz, CDCl₃) δ (ppm): 7.78 (d, 2H), 7.58 (d, 1H), 7.47 (d, 2H), 7.20 (d, 1H), 4.41 (t, 2H), 3.65 (t, 2H), 3.43 (s, 3H), 2.99 (q, 2H), 2.75 (s, 3H), 2.33 (s, 3H), 1.41 (t, 3H); LC-MS: (M + 1) m/z = 474.1. |
| | (19) | |
| | 6-(4-bromophenoxy)-2-ethyl- | |
| | 3,5-dimethylquinolin-4-yl | |
| | (2-methoxyethyl) carbonate | |
| | Example 19 | |
| Intermediate U2 and ethyl 2-methoxychlorofor- mate | 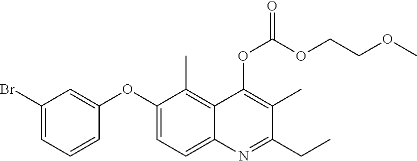 [SMILES: CCc1nc2ccc(Oc3cccc(Br)c3)c(C)c2c(OC(=O)OCCOC)c1C] | yellow liquid; ¹H NMR (400 MHz, CDCl₃) δ (ppm): 7.58 (d, 1H), 7.34 (s, 1H), 7.31 (d, 1H), 7.29 (d, 1H), 7.12 (t, 1H), 7.09 (s, 1H), 4.31 (t, 2H), 3.63 (t, 2H), 3.43 (s, 3H), 2.99 (q, 2H), 2.76 (s, 3H), 2.34 (s, 3H), 1.39 (t, 3H); LC-MS: (M + 1) m/z = 474.1. |
| | (20) | |
| | 6-(3-bromophenoxy)-2-ethyl- | |
| | 3,5-dimethylquinolin-4-yl | |
| | (2-methoxyethyl) carbonate | |
| | Example 20 | |
| Intermediate U3 and ethyl 2-methoxychlorofor- mate | 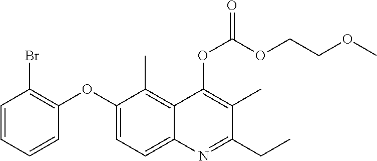 [SMILES: CCc1nc2ccc(Oc3ccccc3Br)c(C)c2c(OC(=O)OCCOC)c1C] | yellow liquid; ¹H NMR (400 MHz, CDCl₃) δ (ppm): 7.61 (d, 1H), 7.59 (d, 1H), 7.28 (t, 1H), 6.99 (d, 1H), 6.96 (d, 1H), 6.89 (t, 1H), 4.33 (t, 2H) 3.61 (t, 2H), 3.44 (s, 3H), 3.01 (q, 2H), 2.77 (s, 3H), 2.34 (s, 3H), 1.31 (t, 3H); LC-MS: (M + 1) m/z = 474.1. |
| | (21) | |
| | 6-(2-bromophenoxy)-2-ethyl- | |
| | 3,5-dimethylquinolin-4-yl | |
| | (2-methoxyethyl) carbonate | |
| | Example 21 | |
| Intermediate U4 and ethyl 2-methoxychlorofor- mate | 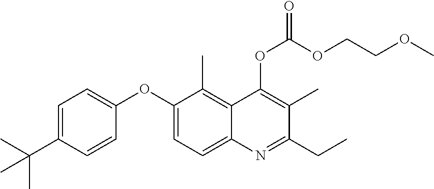 [SMILES: CCc1nc2ccc(Oc3ccc(C(C)(C)C)cc3)c(C)c2c(OC(=O)OCCOC)c1C] | yellow liquid; ¹H NMR (400 MHz, CDCl₃) δ (ppm): 7.59 (d, 2H), 7.55 (d, 1H), 7.31 (d, 2H), 7.25 (d, 1H), 4.39 (t, 2H) 3.66 (t, 2H), 3.41 (s, 3H), 2.99 (q, 2H), 2.76 (s, 3H), 2.29 (s, 3H), 1.44 (s, 9H), 1.41 (t, 3H); LC-MS: (M + 1) m/z = 452.2. |
| | (22) | |
| | 6-(4-(tert-butyl)phenoxy)-2- | |
| | ethyl-3,5-dimethylquinolin-4-yl | |
| | (2-methoxyethyl) carbonate | |
| | Example 22 | |
| Intermediate U5 and ethyl 2-methoxychlorofor- mate | 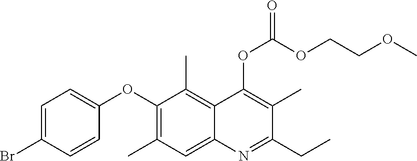 [SMILES: CCc1nc2cc(C)c(Oc3ccc(Br)cc3)c(C)c2c(OC(=O)OCCOC)c1C] | ¹H NMR (400 MHz, CDCl3) δ (ppm): 7.78 (d, 2H), 7.47 (d, 2H), 7.20 (d, 1H), 4.41 (t, 2H), 3.65 (t, 2H), 3.43 (s, 3H), 2.99 (q, 2H), 2.75 (s, 3H), 2.33 (s, 3H), 2.19 (s, 3H), 1.41 (t, 3H); LC-MS: (M + 1) m/z = 488.1. |
| | (23) | |
| | 6-(4-bromophenoxy)-2-ethyl- | |
| | 3,5,7-trimethylquinolin-4-yl | |
| | (2-methoxyethyl) carbonate | |
| | Example 23 | |
| Intermediate U6 and ethyl 2-methoxychlorofor- mate | 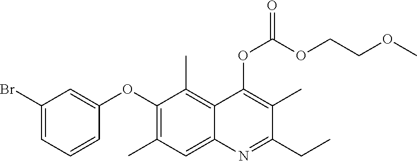 [SMILES: CCc1nc2cc(C)c(Oc3cccc(Br)c3)c(C)c2c(OC(=O)OCCOC)c1C] | yellow liquid; ¹H NMR (400 MHz, CDCl3) δ (ppm): 7.34 (s, 1H), 7.31 (d, 1H), 7.29 (t, 1H), 7.12 (d, 1H), 7.09 (s, 1H), 4.31 (t, 2H), 3.63 (t, 2H), 3.43 (s, 3H), 2.99 (q, 2H), 2.76 (s, 3H), 2.39 (s, 3H), 2.20 (s, 3H), 1.39 (t, 3H); LC-MS: (M + 1) m/z = 488.1. |
| | (24) | |
| | 6-(3-bromophenoxy)-2-ethyl- | |
| | 3,5,7-trimethylquinolin-4-yl | |
| | (2-methoxyethyl) carbonate | |
| | Example 24 | |
| Intermediate U7 and ethyl 2-methoxychlorofor- mate | 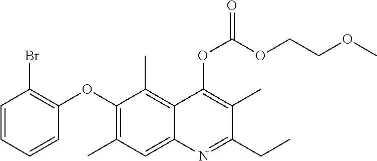 [SMILES: CCc1nc2cc(C)c(Oc3ccccc3Br)c(C)c2c(OC(=O)OCCOC)c1C] | yellow liquid; ¹H NMR (400 MHz, CDCl3) δ (ppm): 7.61 (d, 1H), 7.28 (t, 1H), 6.99 (d, 1H), 6.96 (t, 1H), 6.89 (s, 1H), 4.33 (t, 2H), 3.61 (t, 2H), 3.44 (s, 3H), 3.01 (q, 2H), 2.77 (s, 3H), 2.34 (s, 3H), 2.16 (s, 3H), 1.31 (t, 3H); LC-MS: (M + 1) m/z = 488.1. |
| | (25) | |
| | 6-(2-bromophenoxy)-2-ethyl- | |
| | 3,5,7-trimethylquinolin-4-yl | |
| | (2-methoxyethyl) carbonate | |
| | Example 25 | |
| Intermediate U8 and ethyl 2-methoxychlorofor- mate | 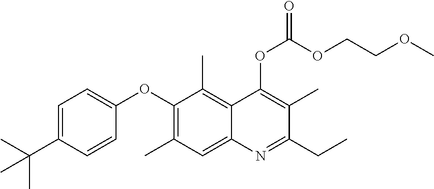 [SMILES: CCc1nc2cc(C)c(Oc3ccc(C(C)(C)C)cc3)c(C)c2c(OC(=O)OCCOC)c1C] | yellow liquid; ¹H NMR (400 MHz, CDCl3) δ (ppm): 7.58 (d, 2H), 7.29 (d, 2H), 6.99 (s, 1H), 4.38 (t, 2H) 3.69 (t, 2H), 3.41 (s, 3H), 2.99 (q, 2H), 2.76 (s, 3H), 2.29 (s, 3H), 2.20 (s, 3H), 1.44 (s, 9H), 1.41 (t, 3H); LC-MS: (M + 1) m/z = 466.3. |
| | (26) | |
| | 6-(4-(tert-butyl)phenoxy)-2- | |
| | ethyl-3,5,7-trimethylquinolin-4- | |
| | yl (2-methoxyethyl) | |
| | carbonate | |
| | Example 26 | |
| Intermediate U9 and 2-methoxychlorofor- mate ethyl | 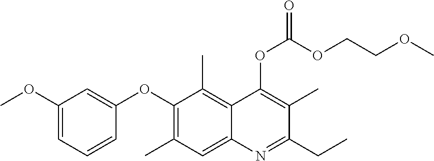 | yellow liquid; ¹H NMR (400 MHz, CDCl3) δ (ppm): 7.34 (t, 1H), 6.99 (s, 1H), 6.71 (d, 1H), 6.65 (s, 1H), 6.61 (d, 1H), 4.31 (t, 2H), 4.02 (s, 3H), 3.63 (t, 2H), 3.43 (s, 3H), 2.99 (q, 2H), 2.76 (s, 3H), 2.35 (s, 3H), 2.20 (s, 3H), 1.33 (t, 3H); LC-MS: (M + 1) m/z = 440.2. |
| | (27) | |
| | 2-ethyl-6-(3-methoxyphenoxy)- | |
| | 3,5,7-trimethylquinolin-4- | |
| | yl (2-methoxyethyl) | |
| | carbonate | |
| | Example 27 | |
| Intermediate U10 and ethyl 2-methoxychlorofor- mate | 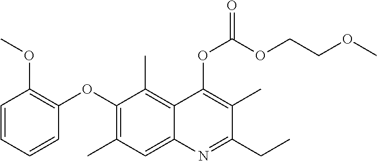 [SMILES: CCc1nc2cc(C)c(Oc3ccccc3OC)c(C)c2c(OC(=O)OCCOC)c1C] | yellow liquid; ¹H NMR (400 MHz, CDCl3) δ (ppm): 6.99 (m, 3H), 6.86 (m, 2H), 4.31 (t, 2H) 4.01 (s, 3H), 3.63 (t, 2H), 3.43 (s, 3H), 2.98 (q, 2H), 2.76 (s, 3H), 2.34 (s, 3H), 2.20 (s, 3H), 1.36 (t, 3H); LC-MS: (M + 1) m/z = 440.2. |
| | (28) | |
| | 2-ethyl-6-(2-methoxyphenoxy)- | |
| | 3,5,7-trimethylquinolin-4- | |
| | yl (2-methoxyethyl) | |
| | carbonate | |
| | Example 28 | |
| Intermediate U11 and ethyl 2-methoxychlorofor- mate | 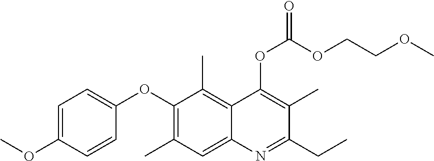 | yellow liquid; ¹H NMR (400 MHz, CDCl3) δ (ppm): 7.35 (d, 2H), 7.09 (d, 2H), 6.89 (s, 1H), 4.31 (t, 2H) 4.01 (s, 3H), 3.63 (t, 2H), 3.43 (s, 3H), 2.98 (q, 2H), 2.76 (s, 3H), 2.34 (s, 3H), 2.20 (s, 3H), 1.36 (t, 3H); LC-MS: (M + 1) m/z = 440.2. |
| | (29) | |
| | 2-ethyl-6-(4-methoxyphenoxy)- | |
| | 3,5,7-trimethylquinolin-4- | |
| | yl (2-methoxyethyl) | |
| | carbonate | |
| | Example 29 | |
| Intermediate U12 and ethyl 2-methoxychlorofor- mate | 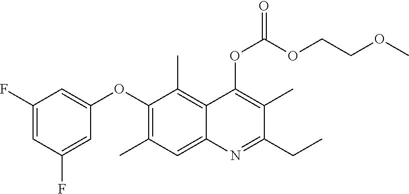 [SMILES: CCc1nc2cc(C)c(Oc3cc(F)cc(F)c3)c(C)c2c(OC(=O)OCCOC)c1C] | yellow liquid; ¹H NMR (400 MHz, CDCl3) δ (ppm): 7.35 (d, 2H), 6.89 (s, 1H), 6.48 (m, 1H), 4.31 (t, 2H) 3.63 (t, 2H), 3.43 (s, 3H), 2.98 (q, 2H), 2.76 (s, 3H), 2.34 (s, 3H), 2.20 (s, 3H), 1.36 (t, 3H); LC-MS: (M + 1) m/z = 446.2. |
| | (30) | |
| | 6-(3,5-difluorophenoxy)-2- | |
| | ethyl-3,5,7-trimethylquinolin-4- | |
| | yl (2-methoxyethyl) | |
| | carbonate | |
| | Example 30 | |
| Intermediate U13 and ethyl 2-methoxychlorofor- mate | 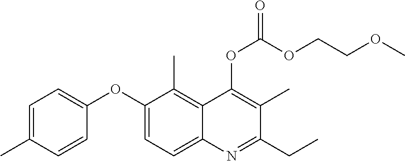 | yellow liquid; ¹H NMR (400 MHz, CDCl3) δ (ppm): 7.55 (d, 1H), 7.19 (d, 2H), 6.89 (d, 2H), 6.87 (s, 1H), 4.31 (t, 2H) 3.63 (t, 2H), 3.43 (s, 3H), 2.98 (q, 2H), 2.34 (s, 3H), 2.28 (s, 3H), 2.20 (s, 3H), 1.36 (t, 3H); LC-MS: (M + 1) m/z = 410.2. |
| | (31) | |
| | 2-ethyl-3,5-dimethyl-6-(p- | |
| | tolyloxy)quinolin-4-yl | |
| | (2-methoxyethyl) carbonate | |
| | Example 31 | |
| Intermediate U14 and ethyl 2-methoxychlorofor- mate | 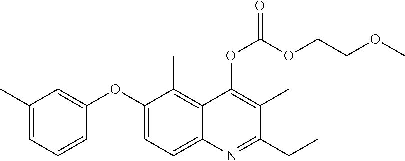 | yellow liquid; ¹H NMR (400 MHz, CDCl3) δ (ppm): 7.58 (d, 1H), 7.29 (t, 1H), 7.18 (s, 1H), 7.06 (d, 1H), 6.85 (d, 1H), 6.81 (d, 1H), 4.31 (t, 2H) 3.63 (t, 2H), 3.43 (s, 3H), 2.97 (q, 2H), 2.34 (s, 3H), 2.29 (s, 3H), 2.20 (s, 3H), 1.35 (t, 3H); LC-MS: (M + 1) m/z = 410.2. |
| | (32) | |
| | 2-ethyl-3,5-dimethyl-6-(m- | |
| | tolyloxy)quinolin-4-yl | |
| | (2-methoxyethyl) carbonate | |
| | Example 32 | |
| Intermediate U15 and ethyl 2-methoxychlorofor- mate | 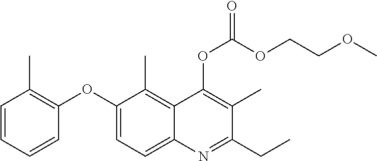 [SMILES: CCc1nc2ccc(Oc3ccccc3C)c(C)c2c(OC(=O)OCCOC)c1C] | yellow liquid; ¹H NMR (400 MHz, CDCl3) δ (ppm): 7.61 (d, 1H), 7.59 (d, 1H), 7.28 (t, 1H), 6.99 (d, 1H), 6.96 (d, 1H), 6.89 (t, 1H), 4.31 (t, 2H) 3.63 (t, 2H), 3.43 (s, 3H), 2.97 (q, 2H), 2.34 (s, 3H), 2.29 (s, 3H), 2.20 (s, 3H), 1.35 (t, 3H); LC-MS: (M + 1) m/z = 410.2. |
| | (33) | |
| | 2-ethyl-3,5-dimethyl-6-(o- | |
| | tolyloxy)quinolin-4-yl | |
| | (2-methoxyethyl) carbonate | |
| | Example 33 | |
| Intermediate U16 and ethyl 2-methoxychlorofor- mate | 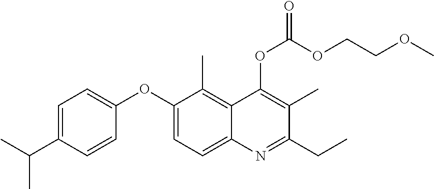 [SMILES: CCc1nc2ccc(Oc3ccc(C(C)C)cc3)c(C)c2c(OC(=O)OCCOC)c1C] | yellow liquid; ¹H NMR (400 MHz, CDCl3) δ (ppm): 7.59 (d, 2H), 7.55 (d, 1H), 7.31 (d, 2H), 7.25 (d, 1H), 4.39 (t, 2H), 3.66 (t, 2H), 3.41 (s, 3H), 2.99 (q, 2H), 2.86 (m, 1H), 2.76 (s, 3H), 2.29 (s, 3H), 1.44 (d, 6H), 1.41 (t, 3H); LC-MS: (M + 1) m/z = 438.2. |
| | (34) | |
| | 2-ethyl-6-(4-isopropylphenoxy)- | |
| | 3,5-dimethylquinolin-4-yl | |
| | (2-methoxyethyl) carbonate | |
| | Example 34 | |
| Intermediate U4 and (tetrahydrofuran-2-yl) methyl chloroformate | 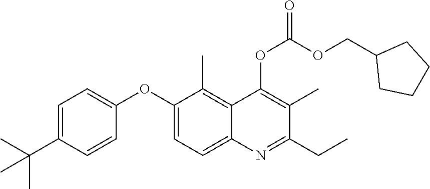 [SMILES: CCc1nc2ccc(Oc3ccc(C(C)(C)C)cc3)c(C)c2c(OC(=O)OCC2CCCC2)c1C] | yellow liquid; ¹H NMR(400 MHz, CDCl3) δ (ppm): 7.66 (s, 1H), 7.58 (d, 2H), 7.39 (d, 2H), 7.21 (s, 1H), 4.48-4.45 (dd, 2H), 4.36-4.34 (m, 1H), 3.89-3.86 (m, 2H), 3.46 (t, 2H), 2.38 (s, H), 2.33 (s, 3H), 1.92-1.89 (m, 2H), 1.73-1.70 (m, 2H), 1.38 (s, 9H), 1.27 (t, 3H); LC-MS: (M + 1) m/z = 478.2. |
| | (35) | |
| | 6-(4-(tert-butyl)phenoxy)-2- | |
| | ethyl-3,5-dimethylquinolin-4-yl | |
| | ((tetrahydrofuran-2-yl) methyl) | |
| | carbonate | |
| | Example 35 | |
| Intermediate U8 and (tetrahydrofuran-2-yl) methyl chloroformate | 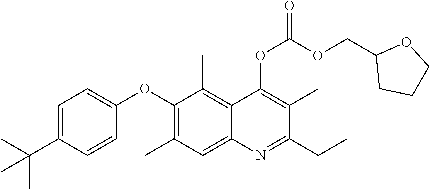 [SMILES: CCc1nc2cc(C)c(Oc3ccc(C(C)(C)C)cc3)c(C)c2c(OC(=O)OCC2CCCO2)c1C] | yellow liquid; ¹H NMR (400 MHz, CDCl3) δ (ppm): 7.56 (d, 2H), 7.42 (s, 1H), 7.35 (d, 2H), 4.52 (dd, 2H), 4.39-4.36 (m, 1H), 3.81-3.79 (m, 2H), 3.42 (q, 2H), 2.38 (s, 3H), 2.34 (s, 3H), 2.17 (s, 3H), 1.93-1.90 (m, 2H), 1.65-1.63 (m, 2H), 1.33 (s, 9H), 1.26 (t, 3H); LC-MS: (M + 1) m/z = 492.2. |
| | (36) | |
| | 6-(4-(tert-butyl)phenoxy)-2- | |
| | ethyl-3,5,7-trimethylquinolin-4- | |
| | yl | |
| | ((tetrahydrofuran-2-yl)methyl) | |
| | carbonate | |
| | Example 36 | |
| 2-ethyl-3,5,7-trimethyl- 6-(4-(trifluoromethoxy) phenoxy)quinolin- 4-ol and (tetrahydrofuran-2-yl) methyl chloroformate | 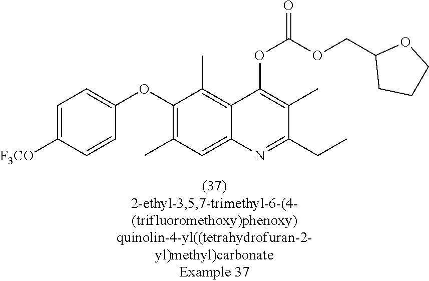 [SMILES: CCc1nc2cc(C)c(Oc3ccc(OC(F)(F)F)cc3)c(C)c2c(OC(=O)OCC2CCCO2)c1C] | yellow liquid; ¹H NMR (400 MHz, CDCl3) δ (ppm): 7.47 (s, 1H), 7.38 (d, 2H), 6.95 (d, 2H), 4.45- 4.42 (m, 2H), 4.37-4.34 (m, 1H), 3.83-3.80 (m, 2H), 3.44- 3.41 (m, 2H), 2.34 (s, 3H), 2.31 (s, 3H), 2.14 (s, 3H), 1.94-1.91 (m, 2H), 1.65-1.62 (m, 2H), 1.28 (t, J = 7.5 Hz, 3H); LC-MS: (M + 1) m/z = 520.2. |
| Intermediate U17 and (tetrahydrofuran-2-yl) methyl chloroformate | 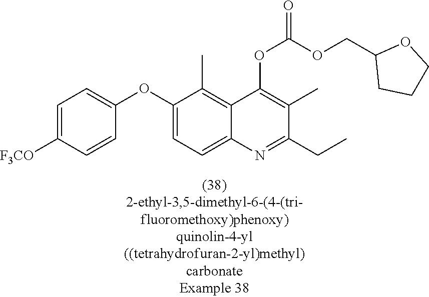 [SMILES: CCc1nc2ccc(Oc3ccc(OC(F)(F)F)cc3)c(C)c2c(OC(=O)OCC2CCCO2)c1C] | yellow liquid; ¹H NMR (400 MHz, CDCl3) δ (ppm): 7.66 (s, 1H), 7.32 (d, 2H), 7.21 (s, 1H), 6.95 (d, 2H), 4.46-4.44 (m, 2H), 4.37-4.34 (m, 1H), 3.85-3.83 (m, 2H), 3.42 (q, 2H), 2.36 (s, 3H), 2.33 (s, 3H), 1.95-1.93 (m, 2H), 1.67-1.64 (m, 2H), 1.24 (t, 3H); LC-MS: (M + 1) m/z = 506.1. |
| Intermediate U17 and ethyl 2-methoxychlorofor- mate | 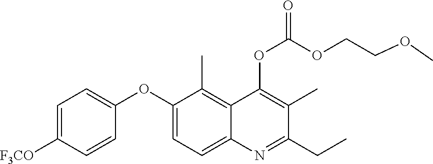 [SMILES: CCc1nc2ccc(Oc3ccc(OC(F)(F)F)cc3)c(C)c2c(OC(=O)OCCOC)c1C] | yellow liquid; ¹H NMR (400 MHz, CDCl3) δ (ppm): 7.69 (s, 1H), 7.38 (d, 2H), 7.19 (s, 1H), 6.95 (d, 2H), 4.31 (t, 2H), 3.67 (t, 2H), 3.45 (q, 2H), 3.32 (s, 3H), 2.36 (s, 3H), 2.33 (s, 3H), 1.27 (t, 3H); LC-MS: (M + 1) m/z = 480.1. |
| | (39) | |
| | 2-ethyl-3,5-dimethyl-6-(4-(tri- | |
| | fluoromethoxy)phenoxy) | |
| | quinolin-4-yl (2-methoxyethyl) | |
| | carbonate | |
| | Example 39 | |
| 2-ethyl-3,5,7-trimethyl- 6-(4-(trifluoromethoxy) phenoxy)quinolin- 4-ol and ethyl 2-ethoxychloroformate | 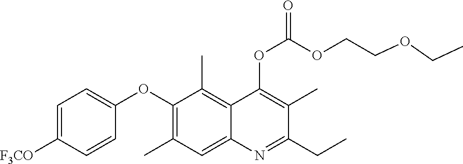 [SMILES: CCOCCOC(=O)Oc1c(C)c(CC)nc2cc(C)c(Oc3ccc(OC(F)(F)F)cc3)c(C)c12] | yellow liquid; ¹H NMR (400 MHz, CDCl3) δ (ppm): 7.49 (s, 1H), 7.35 (d, 2H), 6.98 (d, 2H), 4.32 (d, 2H), 3.67 (d, 2H), 3.53 (q, 2H), 3.46 (q, 2H), 2.34 (s, 3H), 2.31 (s, 3H), 2.15 (s, 3H), 1.24 (t, 3H), 1.18 (t, J = 7.5 Hz, 3H); LC-MS: (M + 1) m/z = 508.1. |
| | (40) | |
| | 2-ethoxyethyl | |
| | (2-ethyl-3,5,7-trimethyl-6-(4- | |
| | (trifluoromethoxy)phenoxy) | |
| | quinolin-4-yl) carbonate | |
| | Example 40 | |
| 2-ethyl-3,5,7-trimethyl- 6-(4-(trifluoromethoxy) phenoxy)quinolin- 4-ol and ethyl 2-isopropoxychloro- formate | 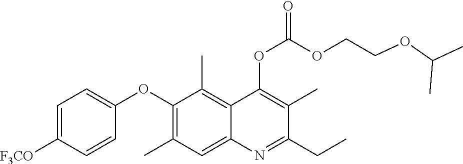 [SMILES: CCc1nc2cc(C)c(Oc3ccc(OC(F)(F)F)cc3)c(C)c2c(OC(=O)OCCOC(C)C)c1C] | yellow liquid; ¹H NMR (400 MHz, CDCl3) δ (ppm): 7.46 (s, 1H), 7.35 (d, 2H), 6.96 (d, 2H), 4.35 (d, 2H), 3.64 (d, 2H), 3.43 (q, J = 7.5 Hz, 2H), 3.14-3.11 (m, 1H), 2.37 (s, 3H), 2.32 (s, 3H), 2.16 (s, 3H), 1.26 (t, 3H), 1.16 (d, 6H); LC-MS: (M + 1) m/z = 522.2. |
| | (41) | |
| | 2-ethyl-3,5,7-trimethyl-6-(4- | |
| | (trifluoromethoxy)phenoxy) | |
| | quinolin-4-yl | |
| | (2-isopropoxyethyl) | |
| | carbonate | |
| | Example 41 | |

### Activity Tests

### 1. Test Examples

1) Preparation of Compound

A certain amount of compound (0.0001 g) was weighed by using an analytical balance, and prepared to a mother liquid (1) with N,N-dimethylformamide containing 1% Tween-80 emulsifier, and then the mother liquid was diluted with distilled water for standby.

2) Test Method

Leaf dipping method: the test target was Armyworm. An appropriate amount of corn leaves were infiltrated in the prepared liquid and dried in the shade naturally, and then placed in a culture dish with filter paper; armyworm larvae in the third instar were inoculated to the corn leaves, 10 heads per dish, and the dishes were cultured in an observation room at 24-27° C. The results were investigated after 3 days. When touching the worm with a brush, it was considered dead if it did not respond. The test concentration was 200 mg/L.

Spraying Leaves and Stems: the test target was Brown rice planthopper. Rice seedlings inoculated with Brown rice planthopper were spayed under Potter spray tower, and then the Brown rice planthopper was placed and cultured in an observation room at 24-27° C. The results were investigated after 72 hours. The test concentration was 500 mg/L.

Leaves Dish Spraying: the test targets were tetranychuscinnabarinus and Aphis craccivora Koch. Vicia faba leaves inoculated with Carmine Spider Mite and Aphis medicaginis were spayed under Potter spray tower, and then the Carmine Spider Mite was placed and cultured in anobservation room at 24-27° C.; the Aphis medicaginis was placed and cultured in an observation room at 20-22° C. The results were investigated after 48 hours. When touching the worm with a brush, it was considered dead if it did not respond. The test concentration was 500 mg/L.

Leaf dipping method: the test targets were Diamondback moth, Tobacco Cutworm and Beet Armyworm. An appropriate amount of cabbage leaves were infiltrated in the prepared liquid and dried in the shade naturally, and then placed in a culture dish with filter paper; worms in the third instar were inoculated to the leaves, 10 heads per dish, and the dishes were cultured in an observation room at 24-27° C. The results were investigated after 3 days. When touching the worm with a brush, it was considered dead if it did not respond. The test concentration was 200 mg/L or 100 mg/L.

The results showed that the mortality rate of example 1 at a concentration of 200 mg/L was 100% to Armyworm and Diamondback moth; the mortality rate of example 1 at a concentration of 100 mg/L was 100% to Beet Armyworm and Tobacco Cutworm; the mortality rate of example 1 at a concentration of 500 mg/L was 100% to Brown rice planthopper and Aphis medicaginis.

### 2. Test Examples

1) Preparation of Compound

A certain amount of compound (0.0001 g) was weighed by using an analytical balance, and prepared to a mother liquid (1%) with N, N-dimethylformamide containing 1% Tween-80 emulsifier, and then the mother liquid was diluted with distilled water for standby.

2) Test Method

Leaf dipping method: an appropriate amount of corn leaves/cabbage/cowpea were infiltrated in the prepared liquid and dried in the shade naturally, and then placed in a culture dish with filter paper; armyworm/Diamondback moth/Beet Armyworm larvae in the 2-3rd instar were inoculated to the corn leaves, 10 heads per dish, and the dishes were cultured in an observation room at 24-27° C. The results were investigated after 3 days. When touching the worm with a brush, it was considered dead if it did not respond. The test concentrations were 50 mg/L, 25 mg/L, 12.5 mg/L, 6.25 mg/L, 3.13 mg/L, 1.56 mg/L.

Spraying: cowpea leaves growing uniformity were punched with a punching bear to make leaf discs, leaf discs were placed onto degreased cotton blocks in a dish, two leaf discs per dish, and water was added into the dish and it was flush with the leaf discs, and the dish was for standby. Frankliniella occidentalis in the 2-3rd instar were inoculated to the ready leaf discs, more than 15 per disc. Which were spayed under Potter spray tower at a dose of 0.5 mL per treatment. The blank control was treated first, and then the above operations were repeated in the order of the test dose from low to high, with 2 repeats per treatment. After treatment, the samples were placed in the observation room, and the results were investigated 1 day later. When touching the worm with a brush, it was considered dead if it did not respond. The test concentrations were 20 mg/L, 10 mg/L, 5.0 mg/L, 2.5 mg/L, 1.25 mg/L, 0.625 mg/L.

The results indicate that the compounds of the invention have high mortality rates to Armyworm, Diamondback moth, Beet Armyworm and Frankliniella occidentalis at different doses.

The results were shown as tables 9 to 12:

TABLE 9
The mortality rate of the compound of the invention to Diamondback
moth at different doses

| | The mortality rate (%) of the compound to | | |
|---|---|---|---|
| | Diamondback moth | | |
| Examples | 50 mg/L | 25 mg/L | 12.5 mg/L |
| Example 1 | 100 | 100 | 100 |
| Example 4 | 100 | 100 | 90 |
| Example 7 | 100 | 90 | 85 |
| Example 8 | 100 | 100 | 93 |
| Example 17 | 100 | 100 | 100 |
| Example 18 | 100 | 100 | 100 |
| Example 22 | 100 | 90 | 85 |
| Example 26 | 100 | 95 | 85 |
| Example 35 | 100 | 90 | 80 |
| Example 36 | 100 | 95 | 85 |
| Example 37 | 100 | 100 | 95 |
| Example 38 | 100 | 100 | 92 |
| Example 39 | 100 | 100 | 100 |
| Example 40 | 100 | 100 | 95 |
| Example 41 | 100 | 100 | 90 |

TABLE 10
The mortality rate of the compound of the invention to
Armyworm at different doses

| | The mortality rate (%) of the compound | | |
|---|---|---|---|
| | to Armyworm | | |
| Examples | 50 mg/L | 25 mg/L | 12.5 mg/L |
| Example 1 | 100 | 100 | 100 |
| Example 4 | 100 | 90 | 80 |
| Example 7 | 100 | 100 | 100 |
| Example 8 | 100 | 100 | 100 |
| Example 17 | 100 | 100 | 100 |
| Example 18 | 100 | 100 | 100 |
| Example 22 | 100 | 95 | 80 |
| Example 26 | 100 | 100 | 90 |
| Example 35 | 100 | 95 | 80 |
| Example 36 | 100 | 100 | 90 |
| Example 37 | 100 | 100 | 100 |
| Example 38 | 100 | 100 | 90 |
| Example 39 | 100 | 100 | 100 |
| Example 40 | 100 | 100 | 95 |
| Example 41 | 100 | 100 | 80 |

TABLE 11
The mortality rate of the compound of the invention to
Beet Armyworm at different doses

| | The mortality rate (%) of the compound | | |
|---|---|---|---|
| | to Beet Armyworm | | |
| Examples | 50 mg/L | 25 mg/L | 12.5 mg/L |
| Example 1 | 100 | 100 | 90 |
| Example 7 | 100 | 100 | 80 |
| Example 8 | 100 | 95 | 70 |
| Example 18 | 100 | 100 | 90 |
| Example 37 | 100 | 100 | 75 |
| Example 38 | 100 | 100 | 70 |

TABLE 12
The mortality rate of the compound of the invention
to Frankliniella occidentalis at different doses

| | The mortality rate (%) of the compound to | | |
|---|---|---|---|
| | Frankliniella occidentalis | | |
| Examples | 20 mg/L | 10 mg/L | 5 mg/L |
| Example 1 | 100 | 100 | 100 |
| Example 7 | 100 | 100 | 95 |
| Example 8 | 100 | 100 | 85 |
| Example 18 | 100 | 100 | 100 |
| Example 37 | 100 | 100 | 95 |
| Example 38 | 100 | 100 | 90 |

In addition, the compounds of the invention still have high mortality rates to the above pests at lower doses; for example, the mortality rate of the compounds of example 1 and example 18 was more than 90% at a dose of 6.25 mg/L; the mortality rate of the compounds of example 1 and example 18 was more than 80% at a dose of 3.13 mg/L.

### 3. Test Examples

1) Preparation of Compound

A certain amount of compound (0.0001 g) was weighed by using an analytical balance, and prepared to a mother liquid (1) with N,N-dimethylformamide containing 1% Tween-80 emulsifier, and then the other liquid was diluted with distilled water or standby.

2) Test Method

Leaf dipping method: an appropriate amount of cabbage was infiltrated in the prepared liquid and dried in the shade naturally, and then placed in a culture dish with filter paper; Armyworm/Diamondback moth larvae in the 2-3rd instar were inoculated, 10 heads per dish, and the dishes were cultured in an observation room at 24-27° C. The results were investigated after 3 days. When touching the worm with a brush, it was considered dead if it did not respond. The test concentrations were 50 mg/L, 25 mg/L, 12.5 mg/L, 6.25 mg/L, 3.13 mg/L, 1.56 mg/L.

The results indicate that compound (42) of the invention have significant control effect on Diamondback moth and Armyworm; specifically, compound (42) have high mortality rates to Diamondback moth and Armyworm at different doses.

The results were shown as tables 13 to 14:

TABLE 13
The mortality rate of the compound of the invention to Diamondback moth at
different doses

| | The mortality rate (%) to | | |
|---|---|---|---|
| | Diamondback moth | | |
| Example | 50 mg/L | 25 mg/L | 12.5 mg/L |
| 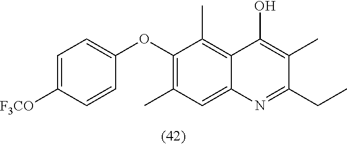 [SMILES: CCc1nc2cc(C)c(Oc3ccc(OC(F)(F)F)cc3)c(C)c2c(O)c1C] | 100 | 100 | 100 |
| 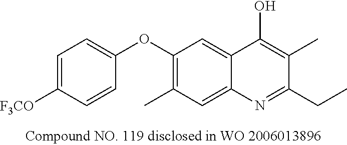 [SMILES: CCc1nc2cc(C)c(Oc3ccc(OC(F)(F)F)cc3)cc2c(O)c1C] | 100 | 90 | 70 |

TABLE 14
The mortality rate of the compound of the invention to Armyworm at different
doses

| | The mortality rate (%) to | | |
|---|---|---|---|
| | Armyworm | | |
| Example | 50 mg/L | 25 mg/L | 12.5 mg/L |
| 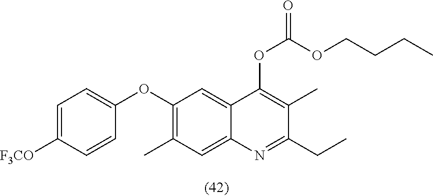 [SMILES: CCCCOC(=O)Oc1c(C)c(CC)nc2cc(C)c(Oc3ccc(OC(F)(F)F)cc3)cc12] | 95 | 85 | 80 |
| 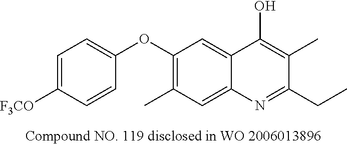 [SMILES: CCc1nc2cc(C)c(Oc3ccc(OC(F)(F)F)cc3)cc2c(O)c1C] | 60 | 50 | 10 |

Wherein compound NO. 119 disclosed in WO 2006013896 can be prepared according to the preparation method of compound (42).

The preferred embodiments of the present invention have been described in detail above, but the present invention is not limited to the details described above, within the scope of the technical concept of the present invention, various simple variants of the technical scheme of the present invention could be made, all of which belong to the scope of the present invention.

1-17. (canceled)

18. A compound of Formula (I) or a stereoisomer, an N-oxide or a salt thereof: 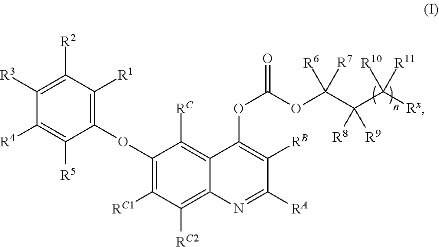
wherein
R^{x} is 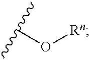
or R¹ and R², or R² and R³, or R³ and R⁴, or R⁴ and R⁵ together form —O—(CH₂)ₘO—, —(CH₂)ₘ₁—O— or —(CH₂)ₘ₂—, wherein each of —O—(CH₂)ₘO—, —(CH₂)ₘ₁—O— and —(CH₂)ₘ₂— is optionally and independently substituted with 1, 2, 3, 4, 5 or 6 halo;
wherein each of m, m1 and m2 is independently 1, 2 or 3;
n is 0, 1, 2 or 3;
Rⁿ is C₁₋₆ alkyl, C₂₋₈ alkenyl, C₂₋₈ alkynyl, C₆₋₁₄ aryl or C₆₋₁₄ aryl-C₁₋₆ alkyl-; wherein Rⁿ is optionally substituted with 1, 2, 3, 4, 5, 6, 7 or 8 substituents selected from A1;
each A1 is independently halo, hydroxy, cyano, nitro, amino, C₁₋₆ alkyl, C₁₋₆ alkoxy, halo C₁₋₆ alkyl or halo C₁₋₆ alkoxy;
each of R¹, R², R³, R⁴ and R⁵ is independently hydrogen, halo, hydroxy, cyano, nitro, amino, carboxy, —C(═O)—NR^{a}R^{b}, —NR^{c}—C(═O)—R^{d}, —NR(OR), C₁₋₆ alkyl, C₂₋₈ alkenyl, C₂₋₈ alkynyl, halo C₁₋₆ alkyl, halo C₂₋₈ alkenyl, halo C₂₋₈ alkynyl, hydroxy-substituted C₁₋₆ alkyl, amino-substituted C₁₋₆ alkyl, cyano-substituted C₁₋₆ alkyl, C₁₋₆ alkyl-SO₂—, C₁₋₆ alkyl-(C═O)—, C₁₋₆ alkyl-(C═O)—O—, C₁₋₆ alkoxy, C₁₋₆ alkoxy-(C═O)—, C₁₋₆ alkylthio, C₂₋₈ alkenyloxy, halo C₁₋₆ alkoxy, halo C₁₋₆ alkylthio, halo C₂₋₈ alkenyloxy, hydroxy-substituted C₁₋₆ alkoxy, amino-substituted C₁₋₆ alkoxy, cyano-substituted C₁₋₆ alkoxy or C₁₋₆ alkylamino;
each of R^{a}, R^{b}, R^{c}, R^{d}, R^{e} and R^{f} is independently hydrogen or C₁₋₆ alkyl; each of R^{A} and R^{B} is independently hydrogen, C₁₋₆ alkyl or halo C₁₋₆ alkyl;
each of R^{C}, R^{C1} and R^{C2} is independently hydrogen, halo or C₁₋₆ alkyl;
each of R⁶, R⁷, R⁸, R⁹, R¹⁰ and R¹¹ is independently hydrogen, C₁₋₄ alkyl, halo C₁₋₄ alkyl, C₁₋₄ alkoxy or halo C₁₋₄ alkoxy;
or Rⁿ and R⁸, together with the atoms to which they are attached, form a 3-8 membered heterocycle; the 3-8 membered heterocycle is optionally substituted with 1, 2, 3, 4, 5, 6, 7 or 8 substituents selected from A3; and
wherein each A3 is independently halo, oxo, hydroxy, cyano, nitro, C₁₋₆ alkyl, C₁₋₆ alkoxy, halo C₁₋₆ alkyl or halo C₁₋₆ alkoxy.

19. The compound of claim 18, wherein
Rⁿ is C₁₋₄ alkyl, C₂₋₄ alkenyl, C₂₋₄ alkynyl, C₆₋₁₀ aryl or C₆₋₁₀ aryl-C₁₋₃ alkyl-; wherein Rⁿ is optionally substituted with 1, 2, 3, 4, 5, 6, 7 or 8 substituents selected from A1; and
each A1 is independently halo, hydroxy, cyano, nitro, amino, C₁₋₅ alkyl, C₁₋₄ alkoxy, halo C₁₋₄ alkyl or halo C₁₋₄ alkoxy.

20. The compound of claim 19, wherein
Rⁿ is —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)CH₃, —C(CH₃)₃, —CH₂F, —CF₂H, —CF₃, —CF₂CHF₂, —CH₂CF₃, —CF₂CHFCF₃, —CH(CF₃)CH₃, —CF(CF₃)₂, —CH₂CH₂—OCH₃ or —CH₂CH₂—OCH₂CH₃;
or Rⁿ is the following sub-structure:

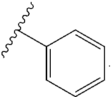 [SMILES: *c1ccccc1]

21. The compound of claim 18, wherein
each of R¹, R², R³, R⁴ and R⁵ is independently hydrogen, halo, hydroxy, cyano, nitro, amino, carboxy, C₁₋₄ alkyl, C₂₋₄ alkenyl, C₂₋₄ alkynyl, halo C₁₋₄ alkyl, halo C₂₋₄ alkenyl, halo C₂₋₄ alkynyl, hydroxy-substituted C₁₋₄ alkyl, amino-substituted C₁₋₄ alkyl, cyano-substituted C₁₋₄ alkyl, C₁₋₄ alkyl-SO₂—, C₁₋₄ alkyl-(C═O)—, C₁₋₄ alkyl-(C═O)—O—, C₁₋₄ alkoxy, C₁₋₄ alkoxy-(C═O)—, C₁₋₄ alkylthio, C₂₋₄ alkenyloxy, halo C₁₋₄ alkoxy, halo C₁₋₄ alkylthio, halo C₂₋₄ alkenyloxy or C₁₋₄ alkylamino.

22. The compound of claim 21, wherein
each of R¹, R², R³, R⁴ and R⁵ is independently hydrogen, fluoro, chloro, bromo, iodo, hydroxy, cyano, nitro, amino, carboxy, —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)CH₃, —C(CH₃)₃, —CH₂F, —CF₂H, —CF₃, —CF₂CHF₂, —CF₂CHFCF₃, —CF(CF₃)₂, —OCH₃, —OCH₂CH₃, —OCH₂CH₂CH₃, —OCH(CH₃)₂, —OC(CH₃)₃, —OCH₂F, —OCF₂H, —OCF₃, —OCF₂CHF₂, —OCH₂CF₃, —OCF₂CHFCF₃, —OCH(CF₃)CH₃ or —OCF(CF₃)₂.

23. The compound of claim 18, wherein
each of R^{A} and R^{B} is independently hydrogen, C₁₋₄ alkyl or halo C₁₋₄ alkyl; and
each of R^{C}, R^{C1} and R^{C2} is independently hydrogen, halo or C₁₋₄ alkyl.

24. The compound of claim 23, wherein
each of R^{A} and R^{B} is independently hydrogen, —CH₃, —CH₂CH₃ or —CHF₂; and
each of R^{C}, R^{C1} and R^{C2} is independently hydrogen, fluoro, chloro, bromo, iodo or —CH₃.

25. The compound of claim 18, wherein
each of R⁶, R⁷, R⁸, R⁹, R¹⁰ and R¹¹ is independently hydrogen, C₁₋₂ alkyl, halo C₁₋₂ alkyl.

26. The compound of claim 18, wherein
Rⁿ and R⁸, together with the atoms to which they are attached, form a 3-6 membered heterocycle; the 3-6 membered heterocycle is optionally substituted with 1, 2, 3, 4, 5, 6, 7 or 8 substituents selected from A3; and
each A3 is independently halo, oxo, hydroxy, cyano, nitro, C₁₋₄ alkyl, C₁₋₄ alkoxy, halo C₁₋₄ alkyl or halo C₁₋₄ alkoxy.

27. The compound of claim 18, wherein
the 3-6 membered heterocycle formed by Rⁿ and R⁸, together with the atoms to which they are attached, is the following sub-structure: 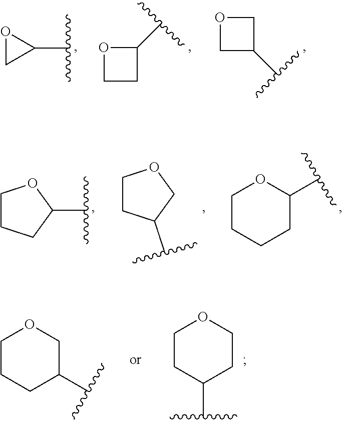
wherein the 3-6 membered heterocycle is optionally substituted with 1, 2, 3, 4, 5, 6, 7 or 8 substituents selected from A3; and
each A3 is independently fluoro, chloro, bromo, iodo, hydroxy, cyano, nitro, —CH₃, —CH₂CH₃, —OCH₃, —OCH₂CH₃, —CF₃ or —OCF₃.

28. The compound of claim 18 having one of the following structures or a stereoisomer, an N-oxide or a salt thereof,

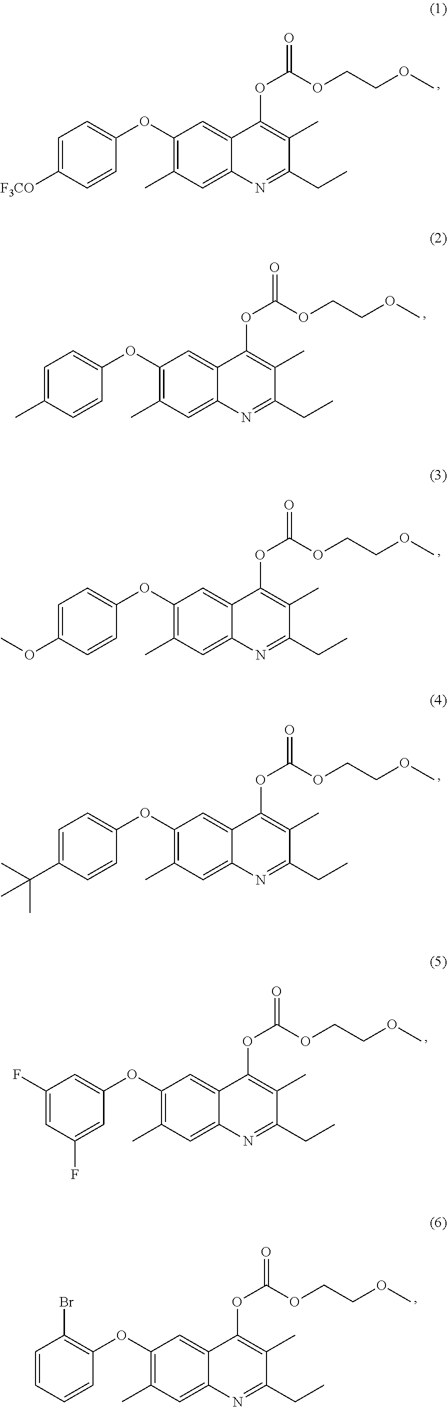

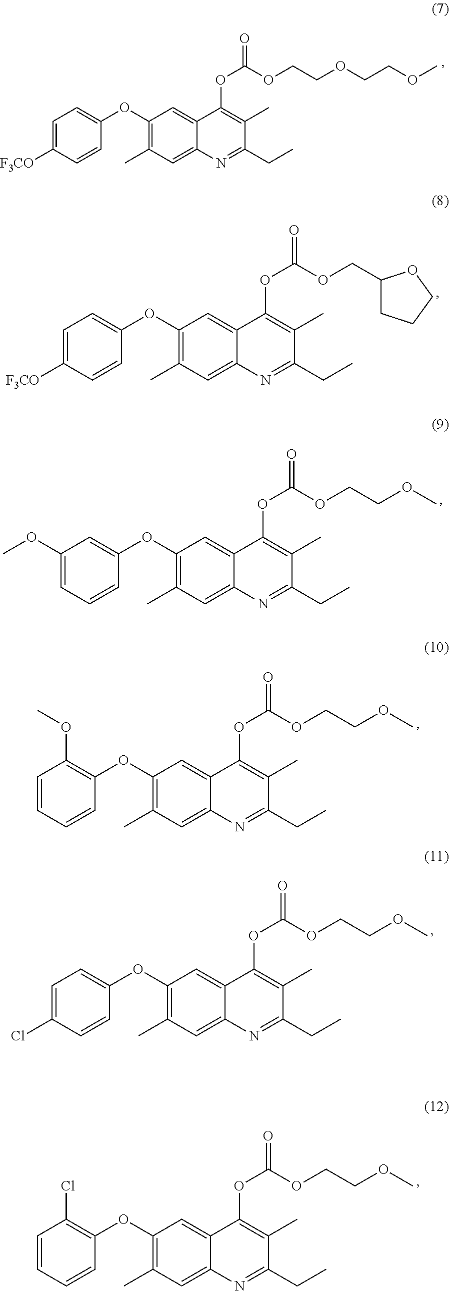

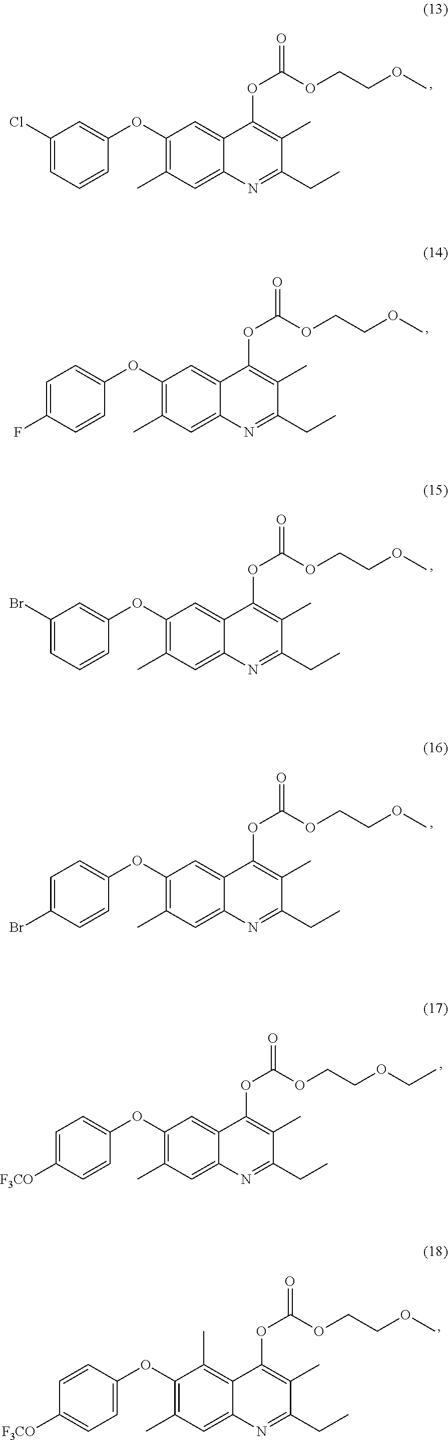 [SMILES: CCc1nc2cc(C)c(Oc3cccc(Cl)c3)cc2c(OC(=O)OCCOC)c1C ; CCc1nc2cc(C)c(Oc3ccc(F)cc3)cc2c(OC(=O)OCCOC)c1C ; CCc1nc2cc(C)c(Oc3cccc(Br)c3)cc2c(OC(=O)OCCOC)c1C ; CCc1nc2cc(C)c(Oc3ccc(Br)cc3)cc2c(OC(=O)OCCOC)c1C ; CCOCCOC(=O)Oc1c(C)c(CC)nc2cc(C)c(Oc3ccc(OC(F)(F)F)cc3)cc12 ; CCc1nc2cc(C)c(Oc3ccc(OC(F)(F)F)cc3)c(C)c2c(OC(=O)OCCOC)c1C]

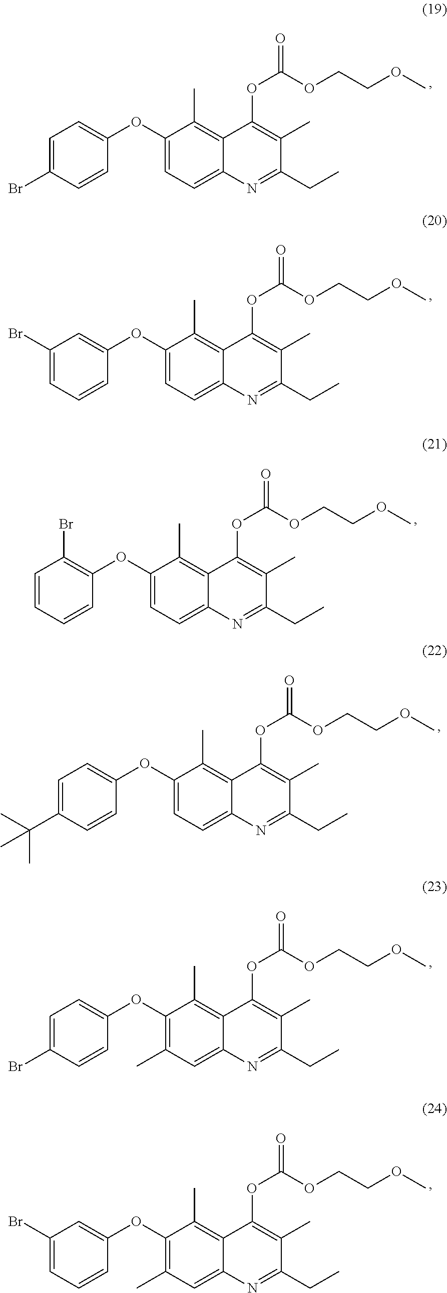

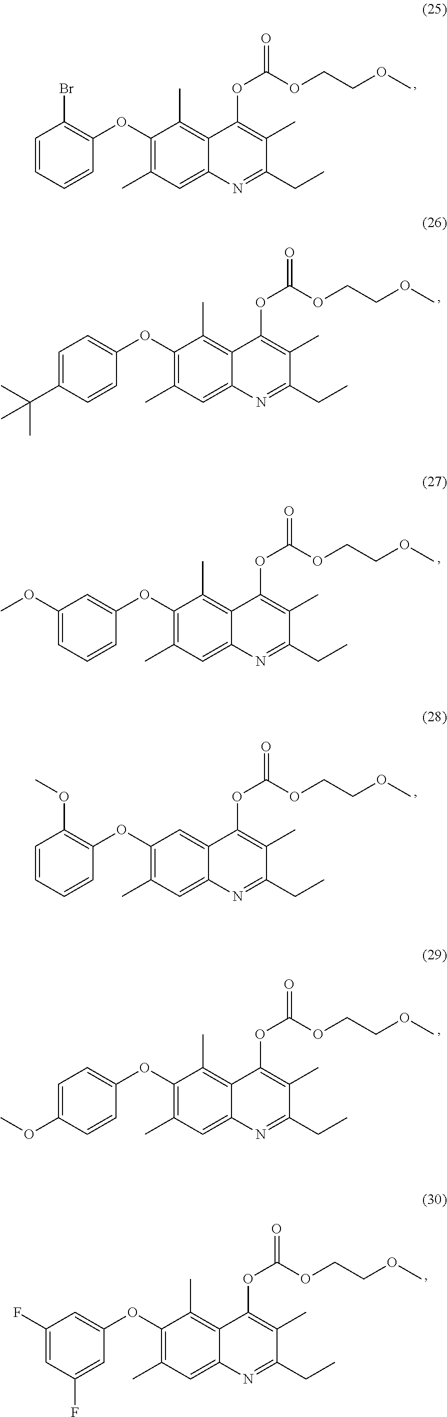

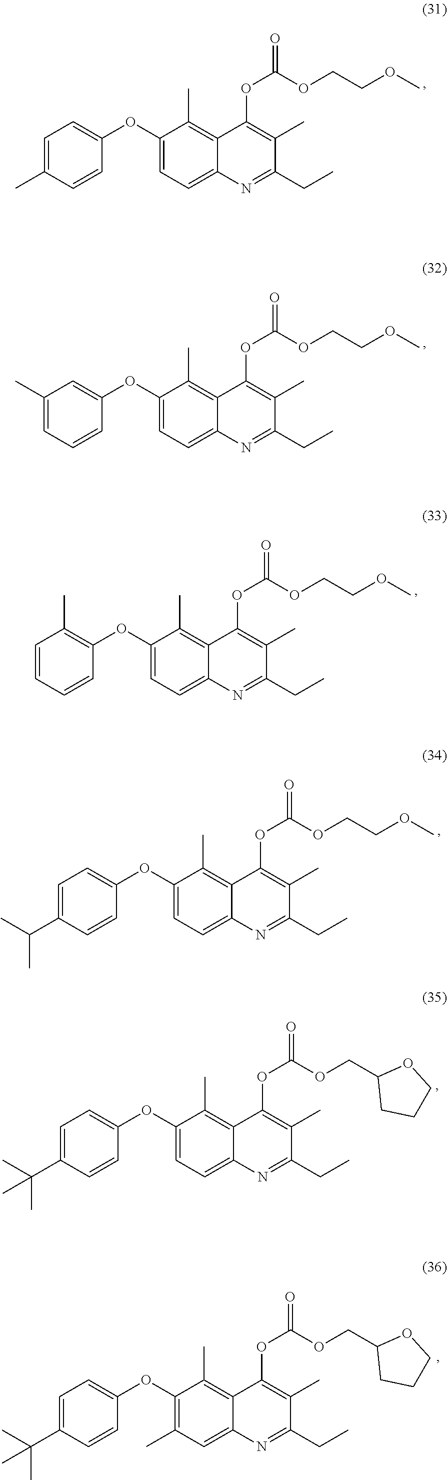

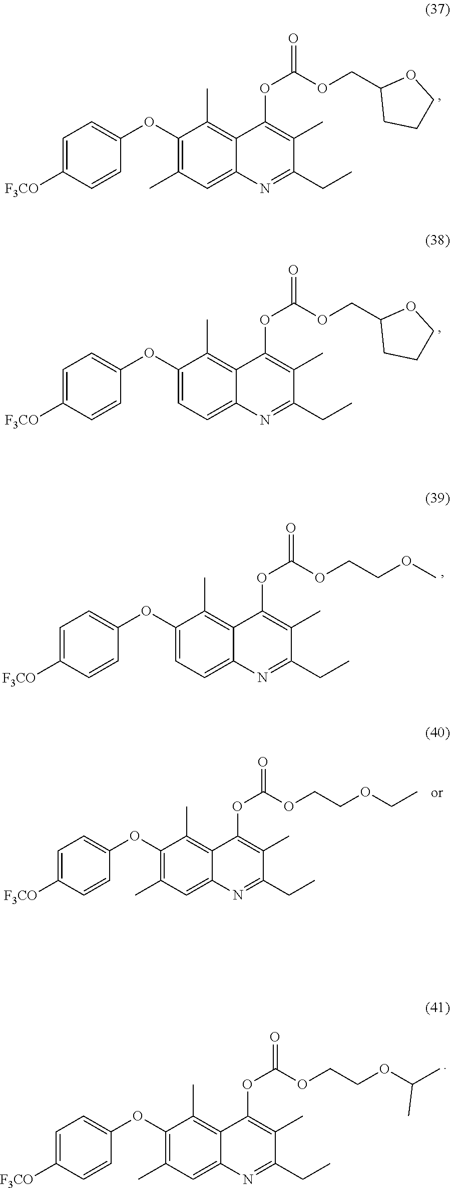 [SMILES: CCc1nc2cc(C)c(Oc3ccc(OC(F)(F)F)cc3)c(C)c2c(OC(=O)OCC2CCCO2)c1C ; CCc1nc2ccc(Oc3ccc(OC(F)(F)F)cc3)c(C)c2c(OC(=O)OCC2CCCO2)c1C ; CCc1nc2ccc(Oc3ccc(OC(F)(F)F)cc3)c(C)c2c(OC(=O)OCCOC)c1C ; CCOCCOC(=O)Oc1c(C)c(CC)nc2cc(C)c(Oc3ccc(OC(F)(F)F)cc3)c(C)c12 ; CCc1nc2cc(C)c(Oc3ccc(OC(F)(F)F)cc3)c(C)c2c(OC(=O)OCCOC(C)C)c1C]

29. A compound of Formula (II) or a stereoisomer, an N-oxide or a salt thereof: 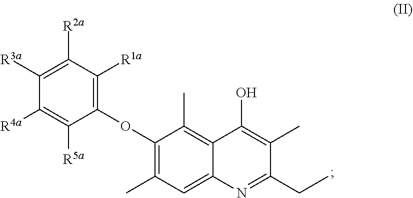
wherein
R^{1a} is hydrogen, fluoro, bromo, iodo, hydroxy, cyano, nitro, amino, carboxy, C₁₋₄ alkyl, halo C₁₋₄ alkyl, C₁₋₄ alkoxy, C₁₋₄ alkylthio, halo C₁₋₄ alkoxy or halo C₁₋₄ alkylthio;
R^{2a} is hydrogen, fluoro, chloro, bromo, iodo, hydroxy, cyano, nitro, amino, carboxy, C₁₋₄ alkyl, halo C₁₋₄ alkyl, C₁₋₄ alkoxy, C₁₋₄ alkylthio, halo C₁₋₄ alkoxy or halo C₁₋₄ alkylthio;
R^{3a} is hydrogen, fluoro, chloro, bromo, iodo, hydroxy, cyano, nitro, amino, carboxy, C₁₋₄ alkyl, halo C₁₋₄ alkyl, C₁₋₄ alkoxy, C₁₋₄ alkylthio, halo C₁₋₄ alkoxy or halo C₁₋₄ alkylthio;
R^{4a} is hydrogen, fluoro, chloro, bromo, iodo, hydroxy, cyano, nitro, amino, carboxy, C₁₋₄ alkyl, halo C₁₋₄ alkyl, C₁₋₄ alkoxy, C₁₋₄ alkylthio, halo C₁₋₄ alkoxy or halo C₁₋₄ alkylthio;
R^{5a} is hydrogen, fluoro, bromo, iodo, hydroxy, cyano, nitro, amino, carboxy, C₁₋₄ alkyl, halo C₁₋₄ alkyl, C₁₋₄ alkoxy, C₁₋₄ alkylthio, halo C₁₋₄ alkoxy or halo C₁₋₄ alkylthio;
with the proviso that the compound of Formula (II) is not 2-ethyl-3,5,7-trimethyl-6-(4-(1,1,2,2-tetrafluoroethoxy)phenoxy)quinolin-4-ol.

30. The compound of claim 29, wherein
R^{1a} is hydrogen, fluoro, bromo, iodo, hydroxy, cyano, nitro, amino, carboxy, —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)CH₃, —C(CH₃)₃, —CH₂F, —CF₂H, —CF₃, —CF₂CHF₂, —CF₂CHFCF₃, —CF(CF₃)₂, —OCH₃, —OCH₂CH₃, —OCH₂CH₂CH₃, —OCH(CH₃)₂, —OC(CH₃)₃, —OCH₂F, —OCF₂H, —OCF₃, —OCH₂CF₃, —OCF₂CHF₂, —OCF₂CHFCF₃, —OCH(CF₃)CH₃ or —OCF(CF₃)₂;
R^{2a} is hydrogen, fluoro, chloro, bromo, iodo, hydroxy, cyano, nitro, amino, carboxy, —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)CH₃, —C(CH₃)₃, —CH₂F, —CF₂H, —CF₃, —CF₂CHF₂, —CF₂CHFCF₃, —CF(CF₃)₂, —OCH₃, —OCH₂CH₃, —OCH₂CH₂CH₃, —OCH(CH₃)₂, —OC(CH₃)₃, —OCH₂F, —OCF₂H, —OCF₃, —OCH₂CF₃, —OCF₂CHF₂, —OCF₂CHFCF₃, —OCH(CF₃)CH₃ or —OCF(CF₃)₂;
R^{3a} is hydrogen, fluoro, chloro, bromo, iodo, hydroxy, cyano, nitro, amino, carboxy, —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)CH₃, —C(CH₃)₃, —CH₂F, —CF₂H, —CF₃, —CF₂CHF₂, —CF₂CHFCF₃, —CF(CF₃)₂, —OCH₃, —OCH₂CH₃, —OCH₂CH₂CH₃, —OCH(CH₃)₂, —OC(CH₃)₃, —OCH₂F, —OCF₂H, —OCF₃, —OCH₂CF₃, —OCF₂CHF₂, —OCF₂CHFCF₃, —OCH(CF₃)CH₃ or —OCF(CF₃)₂;
R^{4a} is hydrogen, fluoro, chloro, bromo, iodo, hydroxy, cyano, nitro, amino, carboxy, —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)CH₃, —C(CH₃)₃, —CH₂F, —CF₂H, —CF₃, —CF₂CHF₂, —CF₂CHFCF₃, —CF(CF₃)₂, —OCH₃, —OCH₂CH₃, —OCH₂CH₂CH₃, —OCH(CH₃)₂, —OC(CH₃)₃, —OCH₂F, —OCF₂H, —OCF₃, —OCH₂CF₃, —OCF₂CHF₂, —OCF₂CHFCF₃, —OCH(CF₃)CH₃ or —OCF(CF₃)₂;
R^{5a} is hydrogen, fluoro, bromo, iodo, hydroxy, cyano, nitro, amino, carboxy, —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)CH₃, —C(CH₃)₃, —CH₂F, —CF₂H, —CF₃, —CF₂CHF₂, —CF₂CHFCF₃, —CF(CF₃)₂, —OCH₃, —OCH₂CH₃, —OCH₂CH₂CH₃, —OCH(CH₃)₂, —OC(CH₃)₃, —OCH₂F, —OCF₂H, —OCF₃, —OCH₂CF₃, —OCF₂CHF₂, —OCF₂CHFCF₃, —OCH(CF₃)CH₃ or —OCF(CF₃)₂;
with the proviso that when R^{1a}, R^{2a}, R^{4a} and R^{5a} are hydrogen, R^{3a} is not —OCF₂CHF₂.

31. The compound of claim 29 having the following structure or a stereoisomer, an N-oxide or a salt thereof,

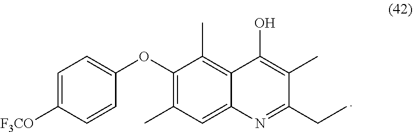 [SMILES: CCc1nc2cc(C)c(Oc3ccc(OC(F)(F)F)cc3)c(C)c2c(O)c1C]

32. A composition comprising the compound of claim 18 and an agriculturally acceptable surfactant and/or carrier.

33. A composition comprising the compound of claim 29 and an agriculturally acceptable surfactant and/or carrier.

34. A method of controlling pests with the compound of claim 18.

35. A method of controlling pests with the compound of claim 29.

36. A method of controlling pests with the composition of claim 32.

37. A method of controlling pests with the composition of claim 33.
